(19) United States
(12) Patent Application Publication
(10) Pub. No.: US 2025/0115965 A1
(43) Pub. Date: Apr. 10, 2025

(54) TARGET-ENRICHED MULTIPLEXED PARALLEL ANALYSIS FOR ASSESSMENT OF TUMOR BIOMARKERS

(71) Applicant: NIPD GENETICS PUBLIC COMPANY LIMITED, Nicosia (CY)

(72) Inventors: George Koumbaris, Lithrodontas (CY); Marios Ioannides, Nicosia (CY); Elena Kypri, Nicosia (CY); Acilleas Achilleos, Limassol (CY); Petros Mina, Nicosia (CY); Alexia Eliades, Nicosia (CY); Charalambos Loizides, Nicosia (CY); Philippos Patsalis, Nicosia (CY)

(21) Appl. No.: 18/926,874

(22) Filed: Oct. 25, 2024

Related U.S. Application Data
(63) Continuation of application No. 16/625,447, filed on Dec. 20, 2019.
(60) Provisional application No. 62/529,779, filed on Jul. 7, 2017.

(51) Int. Cl. C12Q 1/6886; C12N 15/10; C12Q 1/6844; C12Q 1/6874

(52) U.S. Cl. CPC C12Q 1/6886; C12Q 1/6806

(57) ABSTRACT

The invention provides methods for assessment of tumor biomarkers using target-enriched multiplexed parallel analysis. The methods of the invention utilize Target Capture Sequences (TACS) to thereby enrich for target sequences of interest, followed by massive parallel sequencing and statistical analysis of the enriched population. The methods can be used with DNA samples from a patient, such as a tissue biopsy or plasma sample (liquid biopsy), for detection of the presence of tumor biomarkers, e.g., for purposes of diagnosis, screening, therapy selection and/or treatment monitoring. Kits for carrying out the methods of the invention are also provided.

### CROSS REFERENCE TO RELATED PATENT APPLICATIONS

This application is a Continuation of U.S. application Ser. No. 16/625,447, which is a U.S. national phase of International Application No. PCT/EP2018/068441, which was filed on Jul. 6, 2018, and claims priority to U.S. Provisional Application No. 62/529,779, which was filed on Jul. 7, 2017. The content of these earlier filed applications is hereby incorporated by reference herein in its entirety.

### INCORPORATION OF THE SEQUENCE LISTING

The present application contains a sequence listing that was submitted herewith in XML format via Patent Center, containing the file name “37578_0073U2_SL” which is 2,240,053 bytes in size, created on Oct. 24, 2024, and is herein incorporated by reference in its entirety pursuant to 37 C.F.R. § 1.52(e)(5).

### FIELD OF THE INVENTION

The invention is in the field of biology, medicine and chemistry, more in particular in the field of molecular biology and more in particular in the field of molecular diagnostics.

### BACKGROUND OF THE INVENTION

The identification of tumor biomarkers has been an important advance in the detection, diagnosis and treatment of a wide variety of cancers. Various methods of detecting tumor biomarkers are known in the art; however, additional methods are still needed, in particular methods that allow for detection of tumor biomarkers non-invasively, such as in a plasma sample (liquid biopsy). The identification of hereditary (germline) mutations in patients with cancer or high risk individuals suspected of cancer-predisposing syndrome is a useful clinical tool that enables early medical intervention, prophylactic surgery and close monitoring. These germline mutations can be identified in an individual's healthy tissue (such as buccal swab or lymphocytes).

Next generation sequencing (NGS) technologies have been implemented in the development of non-invasive prenatal testing (NIPT). In 2008, two independent groups demonstrated that NIPT of trisomy 21 could be achieved using next generation massively parallel shotgun sequencing (MPSS) (Chiu, R. W. et al. (2008) Proc. Natl. Acad. Sci. USA 105:20458-20463; Fan, H. C. et al. (2008) Proc. Natl. Acad. Sci. USA 105:16266-162710). Large-scale clinical studies using NGS for NIPT have been described (Palomaki, G. E. et al. (2011) Genet. Med. 13:913-920; Ehrich, M. et al. (2011) Am. J. Obstet. Gynecol. 204:205e1-11; Chen, E. Z. et al. (2011) PLoS One 6:e21791; Sehnert, A. J. et al. (2011) Clin. Chem. 57:1042-1049; Palomaki, G. E. et al. (2012); Genet. Med. 14:296-305; Bianchi, D. W. et al. (2012) Obstet. Gynecol. 119:890-901; Zimmerman, B. et al. (2012) Prenat. Diag. 32:1233-1241; Nicolaides, K. H. et al. (2013) Prenat. Diagn. 33:575-579; Sparks, A. B. et al. (2012) Prenat. Diagn. 32:3-9).

Initial NIPT approaches used massively parallel shotgun sequencing (MPSS) NGS methodologies (see e.g., U.S. Pat. Nos. 7,888,017; 8,008,018; 8,195,415; 8,296,076; 8,682,594; US Patent Publication 20110201507; US Patent Publication 20120270739). Thus, these approaches are whole genome-based. More recently, targeted-based NGS approaches for NIPT, in which only specific sequences of interest are sequenced, have been developed. For example, a targeted NIPT approach using TArget Capture Sequences (TACS) for identifying fetal chromosomal abnormalities using a maternal blood sample has been described (PCT Publication WO 2016/189388; US Patent Publication 2016/0340733; Koumbaris, G. et al. (2016) Clinical chemistry, 62(6), pp.848-855.). Such targeted approaches require significantly less sequencing than the MPSS approaches, since sequencing is only performed on specific loci on the target sequence of interest rather than across the whole genome.

Additional methodologies for NGS-based approaches are still needed, in particular approaches that can target specific sequences of interest, such as for example tumor biomarkers, thereby greatly reducing the amount of sequencing needed as compared to whole genome-based approaches, as well as increasing the read-depth of regions of interest, thus enabling detection of low signal to noise ratio regions. In particular, additional methodologies are still needed that allow for genetic aberrations present in diminutive amounts in a sample to be reliably detected, such as for example in the early detection of cancer.

### SUMMARY OF THE INVENTION

This invention provides improved methods for enriching targeted genomic regions of interest to be analyzed by multiplexed parallel sequencing, wherein the enriched sequences are tumor biomarker sequences and the DNA sample used in the method is from a subject having or suspected of having a tumor. Accordingly, the methods allow for detection of tumor biomarkers in a variety of biological samples, including liquid samples, such as plasma samples (liquid biopsy), thereby providing non-invasive means for tumor detection and monitoring. The methods of the invention utilize a pool of TArget Capture Sequences (TACS) designed such that the sequences within the pool have features that optimize the efficiency, specificity and accuracy of genetic assessment of tumor biomarkers. The methods of the invention can be used, for example, in cancer diagnosis, cancer screening, cancer treatment regimen selection and/or cancer therapy monitoring.

Accordingly, in one aspect, the invention pertains to a method of detecting one or more tumor biomarkers in a DNA sample from a subject having or suspected of having a tumor, the method comprising:
(a) preparing a sequencing library from the DNA sample;
(b) hybridizing the sequencing library to a pool of double-stranded TArget Capture Sequences (TACS) that bind to one or more tumor biomarker sequences of interest, wherein:
   (i) each member sequence within the pool of TACS is between 100-500 base pairs in length, each member sequence having a 5′ end and a 3′ end;
   (ii) preferably each member sequence binds to the tumor biomarker sequence of interest at least 50 base pairs away, on both the 5′ end and the 3′ end, from regions harboring Copy Number Variations (CNVs), Segmental duplications or repetitive DNA elements; and
   (iii) the GC content of the pool of TACS is between 19% and 80%, as determined by calculating the GC content of each member within the pool of TACS;
(c) isolating members of the sequencing library that bind to the pool of TACS to obtain an enriched library;
(d) amplifying and sequencing the enriched library; and
(e) performing statistical analysis on the enriched library sequences, optionally utilizing only fragments of a specific size range, to thereby detect the tumor biomarker(s) in the DNA sample.

In one embodiment, the pool of TACS comprises a plurality of TACS families, wherein each member of a TACS family binds to the same tumor biomarker sequence of interest but with different start and/or stop positions on the sequence with respect to a reference coordinate system (i.e., binding of TACS family members to the target sequence is staggered) to thereby enrich for target sequences of interest, followed by massive parallel sequencing and statistical analysis of the enriched population. The use of families of TACS with the TACS pool that bind to each target sequence of interest, as compared to use of a single TACS within the TACS pool that binds to each target sequence of interest, significantly increases enrichment for the target sequences of interest, as evidenced by a greater than 50% average increase in read-depth for the family of TACS versus a single TACS. Herein, the mutations detected or biomarkers detected may be due to somatic mutation or may be hereditary, i.e already present in the germ line.

Accordingly, in one embodiment, the pool of TACS comprises a plurality of TACS families directed to different tumor biomarker sequences of interest, wherein each TACS family comprises a plurality of member sequences, wherein each member sequence binds to the same tumor biomarker sequence of interest but has different start and/or stop positions with respect to a reference coordinate system for the genomic sequence of interest.

In certain embodiments, each TACS family comprises at least 3 member sequences or at least 5 member sequences. Alternative numbers of member sequences in each TACS family are described herein. In one embodiment, the pool of TACS comprises at least 50 different TACS families. Alternative numbers of different TACS families within the pool of TACS are described herein. In certain embodiments, the start and/or stop positions for the member sequences within a TACS family, with respect to a reference coordinate system for the genomic sequence of interest, are staggered by at least 3 base pairs or at least 5 base pairs or by at least 10 base pairs. Alternative lengths (sizes) for the number of base pairs within the stagger are described herein.

In one embodiment, each member sequence within the pool of TACS is at least 160 base pairs in length. In certain embodiments, the GC content of the pool of TACS is between 19% and 80% or is between 19% and 46%. Alternative % ranges for the GC content of the pool of TACS are described herein.

In one embodiment, the pool of TACS is fixed to a solid support. For example, in one embodiment, the TACS are biotinylated and are bound to streptavidin-coated magnetic beads.

In one embodiment, amplification of the enriched library is performed in the presence of blocking sequences that inhibit amplification of wild-type sequences.

In one embodiment, members of the sequencing library that bind to the pool of TACS are partially complementary to the TACS.

In one embodiment, the statistical analysis comprises a segmentation algorithm, for example, likelihood-based segmentation, segmentation using small overlapping windows, segmentation using parallel pairwise testing, and combinations thereof. In one embodiment, the statistical analysis comprises a score-based classification system. In one embodiment, sequencing of the enriched library provides a read-depth for the genomic sequences of interest and read-depths for reference loci and the statistical analysis comprises applying an algorithm that tests sequentially the read-depth of the loci of from the genomic sequences of interest against the read-depth of the reference loci, the algorithm comprising steps for: (a) removal of inadequately sequenced loci; (b) GC-content bias alleviation; and (c) genetic status determination. In one embodiment, GC-content bias is alleviated by grouping together loci of matching GC content. In one embodiment, sequencing of the enriched library provides the number and size of sequenced fragments for TACS-specific coordinates and the statistical analysis comprises applying an algorithm that tests sequentially the fragment-size proportion for the genomic sequence of interest against the fragment-size proportion of the reference loci, the algorithm comprising steps for: (a) removal of fragment-size outliers; (b) fragment-size proportion calculation; and (c) genetic status determination.

In one embodiment, the DNA sample comprises cell free tumor DNA (cftDNA). In various embodiments, the DNA sample is selected from a group comprising of a plasma sample, a urine sample, a sputum sample, a cerebrospinal fluid sample, an ascites sample and a pleural effusion sample from subject having or suspected of having a tumor. In one embodiment, the DNA sample is from a tissue sample from a subject having or suspected of having a tumor.

In one embodiment, the pool of TACS binds to a plurality of tumor biomarker sequences of interest selected from a group comprising ABL, AKT, AKT1, ALK, APC, AR, ARAF, ATM, BAP1, BARD1, BCL, BMPR1A, BRAF, BRCA, BRCA1, BRCA2, BRIP1, CDH1, CDKN, CHEK2, CTNNB1, DDB2, DDR2, DICER1, EGFR, EPCAM, ErbB, ErcC, ESR1, FANCA, FANCB, FANCC, FANCD2, FANCE, FANCF, FANCG, FANCI, FANCL, FANCM, FBXW7, FGFR, FLT, FLT3, FOXA1, FOXL2, GATA3, GNA11, GNAQ, GNAS, GREM1, HOX, HOXB13, HRAS, IDH1, JAK, JAK2, KEAP1, KIT, KRAS, MAP2Ks, MAP3Ks, MET, MLH1, MPL, MRE11A, MSH2, MSH6, MTOR, MUTYH, NBN, NPM1, NRAS, NTRK1, PALB2, PDGFRs, PI3KCs, PMS2, POLD1, POLE, POLH, PTEN, RAD50, RAD51C, RAD51D, RAF1, RB1, RET, RUNX1, SLX4, SMAD, SMAD4, SMARCA4, SPOP, STAT, STK11, TP53, VHL, XPA and XPC, and combinations thereof.

In another embodiment, the pool of TACS binds to a plurality of tumor biomarker sequences of interest selected from a group comprising, AKT1, ALK, APC, AR, ARAF, ATM, BAP1, BARD1, BMPR1A, BRAF, BRCA1, BRCA2, BRIP1, CDH1, CDK4, CDKN2A (p14ARF), CDKN2A (p16INK4a), CHEK2, CTNNB1, DDB2, DDR2, DICER1, EGFR, EPCAM, ERBB2, ERBB3, ERBB4, ERCC1, ERCC2, ERCC3, ERCC4, ERCC5, ESR1, FANCA, FANCB, FANCC, FANCD2, FANCE, FANCF, FANCG, FANCI, FANCL, FANCM, FBXW7, FGFR1, FGFR2, FLT3, FOXA1, FOXL2, GATA3, GNA11, GNAQ, GNAS, GREM1, HOXB13, IDH1, IDH2, JAK2, KEAP1, KIT, KRAS, MAP2K1, MAP3K1, MEN1, MET, MLH1, MPL, MRE11A, MSH2, MSH6, MTOR, MUTYH, MYC, MYCN, NBN, NPM1, NRAS, NTRK1, PALB2, PDGFRA, PIK3CA, PIK3CB, PMS2, POLD1, POLE, POLH, PTEN, RAD50, RAD51C, RAD51D, RAF1, RB1, RET, ROS1, RUNX1, SDHA, SDHAF2, SDHB, SDHC, SDHD, SLX4, SMAD4, SMARCA4, SPOP, STAT, STK11, TMPRSS2, TP53, VHL, XPA, XPC and combinations thereof.

In one embodiment, the pool of TACS binds to a plurality of tumor biomarker sequences of interest selected from a group comprising EGFR_6240, KRAS_521, EGFR_6225, NRAS_578, NRAS_580, PIK3CA_763, EGFR_13553, EGFR_18430, BRAF_476, KIT_1314, NRAS_584, EGFR_12378, and combinations thereof.

In another embodiment, the pool of TACS binds to a plurality of tumor biomarker sequences of interest selected from a group comprising COSM6240 (EGFR_6240), COSM521 (KRAS_521), COSM6225 (EGFR_6225), COSM578 (NRAS_578), COSM580 (NRAS_580), COSM763 (PIK3CA_763), COSM13553 (EGFR_13553), COSM18430 (EGFR_18430), COSM476 (BRAF_476), COSM1314 (KIT_1314), COSM584 (NRAS_584), COSM12378 (EGFR_12378), and combinations thereof, wherein the identifiers refer to the COSMIC database ID number of the biomarker.

In one embodiment, the method further comprises making a diagnosis of the subject based on detection of at least one tumor biomarker sequence. In another embodiment, the method further comprises selecting a therapeutic regimen for the subject based on detection of at least one tumor biomarker sequence. In yet another embodiment, the method further comprises monitoring treatment efficacy of a therapeutic regimen in the subject based on detection of at least one tumor biomarker sequence.

In another aspect, kits for performing the methods of the invention are also encompassed.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a schematic diagram of multiplexed parallel analysis of targeted genomic regions for non-invasive prenatal testing using TArget Capture Sequences (TACS).

FIGS. 2A-2CB are a listing of exemplary chromosomal regions for amplifying TACS that bind to for example chromosomes 13, 18, 21 or X. A more extensive list is shown in Table 1 below.

FIG. 3 is a schematic diagram of TACS-based enrichment of a sequence of interest (bold line) using a single TACS (left) versus TACS-based enrichment using a family of TACS (right).

FIGS. 4A-4B are graphs showing enrichment using families of TACS versus a single TACS, as illustrated by increase in the average read-depth. FIG. 4A shows loci enriched using a family of TACS (red dots) as compared to loci enriched using a single TACS (blue dots), with different target sequences shown on the x-axis and the fold change in read-depth shown on the y-axis. FIG. 4B is a bar graph illustrating the average fold-increase in read-depth (54.7%) using a family of TACS (right) versus a single TACS (left).

FIG. 5 shows bar graphs illustrating detection of known genetic mutations that are tumor biomarkers in certified reference material harboring the mutations. Two replicates of the reference material are shown. The line illustrates the expected minor allele frequency (MAF) for each of the assessed tumor loads. The bars (x-axis) illustrate the detected MAF (y-axis) for the indicated genetic mutations in the certified reference material.

FIG. 6 shows bar graphs illustrating detection of tumor biomarkers in cancer patient samples. Results are shown for two patients, one harboring mutation PIK3CA E545K (top bars) and one harboring mutation TP53 K139 (bottom bars). Both tumor tissue samples (“Tissue Rep. 1” and “Tissue Rep. 2”) and plasma samples (“Plasma”) are shown. The y-axis shows % variant allele frequency (VAF) detected in the samples.

FIG. 7 is a bar graph showing the observed pattern of somatic SNVs in breast cancer, as found in the COSMIC database. The x-axis shows a single base mutation observed in cancer in the context of its neighboring sequences. For example A[C>A]T describes the mutation of Cytosine (C) to Adenine (A) where the upstream sequence is Adenine and the downstream sequence is Thymine. The y-axis shows the frequency of occurrence of this mutation in breast cancer.

FIG. 8 is a bar graph showing results of a simulations study where simulated sequencing data includes mutational motifs. The data were subjected to mutational motif detection. The bars indicate the average estimated frequency of the known mutational breast cancer motifs computed from a data set of 10000 simulations. Results illustrate that detection of mutational motifs is possible using the developed algorithm.

FIG. 9 is a dot plot graph showing results of a fragments-based test for detecting increased numbers of smaller-size fragments in a mixed sample. An abnormal, aneuploid sample, with an estimated fetal fraction of 2.8%, was correctly detected using this method. The black dots are individual samples. The x-axis shows the sample index. The y-axis shows the score result of the fragments-size based method. A score result greater than the threshold shown by the grey line indicates a deviation from the expected size of fragments illustrating the presence of aneuploidy.

FIG. 10 is a listing of exemplary chromosomal regions for amplifying TACS that bind to exemplary, non-limiting tumor biomarker genes.

Table 1 shows exemplary and preferred TACS positions.

| Chr. | Start | Stop | GC | Gene |
|---|---|---|---|---|
| chr1 | 11169250 | 11169491 | 0.434 | MTOR |
| chr1 | 11169262 | 11169509 | 0.419 | MTOR |
| chr1 | 11169280 | 11169519 | 0.400 | MTOR |
| chr1 | 11169299 | 11169548 | 0.392 | MTOR |
| chr1 | 11174376 | 11174632 | 0.541 | MTOR |
| chr1 | 11174392 | 11174632 | 0.535 | MTOR |
| chr1 | 11174392 | 11174691 | 0.527 | MTOR |
| chr1 | 11174468 | 11174698 | 0.515 | MTOR |
| chr1 | 11184541 | 11184796 | 0.504 | MTOR |
| chr1 | 11184563 | 11184812 | 0.504 | MTOR |
| chr1 | 11184564 | 11184816 | 0.502 | MTOR |
| chr1 | 11187992 | 11188236 | 0.535 | MTOR |
| chr1 | 11188010 | 11188249 | 0.521 | MTOR |
| chr1 | 11188018 | 11188257 | 0.513 | MTOR |
| chr1 | 11188029 | 11188274 | 0.492 | MTOR |
| chr1 | 17345194 | 17345459 | 0.316 | SDHB |
| chr1 | 17349096 | 17349342 | 0.543 | SDHB |
| chr1 | 17350413 | 17350563 | 0.497 | SDHB |
| chr1 | 17350566 | 17350779 | 0.430 | SDHB |
| chr1 | 17354089 | 17354304 | 0.463 | SDHB |
| chr1 | 17355058 | 17355208 | 0.417 | SDHB |
| chr1 | 17359477 | 17359689 | 0.427 | SDHB |
| chr1 | 17371214 | 17371394 | 0.470 | SDHB |
| chr1 | 17380408 | 17380619 | 0.670 | SDHB |
| chr1 | 43814917 | 43815186 | 0.633 | MPL |
| chr1 | 45795001 | 45795250 | 0.536 | MUTYH |
| chr1 | 45796024 | 45796223 | 0.530 | MUTYH |
| chr1 | 45796871 | 45797092 | 0.536 | MUTYH |
| chr1 | 45797060 | 45797289 | 0.648 | MUTYH |
| chr1 | 45797289 | 45797529 | 0.635 | MUTYH |
| chr1 | 45797602 | 45797802 | 0.577 | MUTYH |
| chr1 | 45797819 | 45798019 | 0.622 | MUTYH |
| chr1 | 45797986 | 45798270 | 0.593 | MUTYH |
| chr1 | 45798204 | 45798404 | 0.602 | MUTYH |
| chr1 | 45798364 | 45798564 | 0.547 | MUTYH |
| chr1 | 45798532 | 45798732 | 0.557 | MUTYH |
| chr1 | 45798672 | 45798872 | 0.567 | MUTYH |
| chr1 | 45798867 | 45799097 | 0.628 | MUTYH |
| chr1 | 45799150 | 45799357 | 0.582 | MUTYH |
| chr1 | 45800062 | 45800304 | 0.539 | MUTYH |
| chr1 | 115252133 | 115252352 | 0.445 | NRAS |
| chr1 | 115252133 | 115252354 | 0.441 | NRAS |
| chr1 | 115252138 | 115252350 | 0.446 | NRAS |
| chr1 | 115252142 | 115252350 | 0.450 | NRAS |
| chr1 | 115256347 | 115256588 | 0.409 | NRAS |
| chr1 | 115256398 | 115256647 | 0.448 | NRAS |
| chr1 | 115256410 | 115256649 | 0.458 | NRAS |
| chr1 | 115256442 | 115256691 | 0.440 | NRAS |
| chr1 | 115256467 | 115256726 | 0.442 | NRAS |
| chr1 | 115256470 | 115256715 | 0.451 | NRAS |
| chr1 | 115258538 | 115258778 | 0.440 | NRAS |
| chr1 | 115258570 | 115258813 | 0.463 | NRAS |
| chr1 | 115258607 | 115258846 | 0.492 | NRAS |
| chr1 | 115258659 | 115258901 | 0.444 | NRAS |
| chr1 | 156268500 | 156268651 | 0.454 | VHLL |
| chr1 | 156269007 | 156269221 | 0.493 | VHLL |
| chr1 | 156846129 | 156846362 | 0.641 | NTRK1 |
| chr1 | 156846129 | 156846377 | 0.643 | NTRK1 |
| chr1 | 156846130 | 156846362 | 0.639 | NTRK1 |
| chr1 | 156848889 | 156849132 | 0.611 | NTRK1 |
| chr1 | 156848911 | 156849153 | 0.617 | NTRK1 |
| chr1 | 156848921 | 156849168 | 0.613 | NTRK1 |
| chr1 | 156848925 | 156849168 | 0.611 | NTRK1 |
| chr1 | 161284168 | 161284376 | 0.598 | SDHC |
| chr1 | 161293229 | 161293429 | 0.343 | SDHC |
| chr1 | 161298181 | 161298414 | 0.436 | SDHC |
| chr1 | 161333007 | 161333261 | 0.447 | SDHC |
| chr1 | 161333275 | 161333550 | 0.391 | SDHC |
| chr1 | 161333589 | 161333868 | 0.439 | SDHC |
| chr1 | 161333890 | 161334157 | 0.392 | SDHC |
| chr1 | 161334206 | 161334492 | 0.394 | SDHC |
| chr1 | 162748235 | 162748450 | 0.440 | DDR2 |
| chr1 | 162748273 | 162748512 | 0.442 | DDR2 |
| chr1 | 162748316 | 162748557 | 0.450 | DDR2 |
| chr1 | 162748370 | 162748580 | 0.483 | DDR2 |
| chr10 | 8111358 | 8111588 | 0.506 | GATA3 |
| chr10 | 8111468 | 8111707 | 0.529 | GATA3 |
| chr10 | 8115737 | 8115986 | 0.572 | GATA3 |
| chr10 | 8115741 | 8115988 | 0.569 | GATA3 |
| chr10 | 8115783 | 8115988 | 0.612 | GATA3 |
| chr10 | 8115789 | 8115988 | 0.620 | GATA3 |
| chr10 | 43595933 | 43596179 | 0.623 | RET |
| chr10 | 43597765 | 43598054 | 0.614 | RET |
| chr10 | 43600476 | 43600682 | 0.686 | RET |
| chr10 | 43601846 | 43602088 | 0.638 | RET |
| chr10 | 43604444 | 43604706 | 0.620 | RET |
| chr10 | 43606653 | 43606911 | 0.595 | RET |
| chr10 | 43607465 | 43607728 | 0.659 | RET |
| chr10 | 43608226 | 43608455 | 0.596 | RET |
| chr10 | 43608980 | 43609217 | 0.626 | RET |
| chr10 | 43609917 | 43610183 | 0.625 | RET |
| chr10 | 43611943 | 43612187 | 0.522 | RET |
| chr10 | 43613689 | 43613933 | 0.555 | RET |
| chr10 | 43614921 | 43615150 | 0.687 | RET |
| chr10 | 43615517 | 43615759 | 0.568 | RET |
| chr10 | 43617287 | 43617531 | 0.457 | RET |
| chr10 | 43619092 | 43619322 | 0.584 | RET |
| chr10 | 43620303 | 43620509 | 0.551 | RET |
| chr10 | 43623746 | 43624029 | 0.493 | RET |
| chr10 | 43624152 | 43624439 | 0.490 | RET |
| chr10 | 43624946 | 43625222 | 0.433 | RET |
| chr10 | 50686414 | 50686655 | 0.368 | ERCC6 |
| chr10 | 88635669 | 88635923 | 0.333 | BMPR1A |
| chr10 | 88649829 | 88650073 | 0.384 | BMPR1A |
| chr10 | 88659444 | 88659644 | 0.368 | BMPR1A |
| chr10 | 88677042 | 88677242 | 0.388 | BMPR1A |
| chr10 | 89622844 | 89623045 | 0.594 | KLLN |
| chr10 | 89624131 | 89624370 | 0.492 | PTEN |
| chr10 | 89624178 | 89624422 | 0.465 | PTEN |
| chr10 | 89624198 | 89624444 | 0.445 | PTEN |
| chr10 | 89624214 | 89624463 | 0.436 | PTEN |
| chr10 | 89685273 | 89685522 | 0.304 | PTEN |
| chr10 | 89692746 | 89692946 | 0.398 | PTEN |
| chr10 | 89692746 | 89692977 | 0.392 | PTEN |
| chr10 | 89692746 | 89692999 | 0.402 | PTEN |
| chr10 | 89692763 | 89693015 | 0.403 | PTEN |
| chr10 | 89692787 | 89692999 | 0.413 | PTEN |
| chr10 | 89711788 | 89712024 | 0.380 | PTEN |
| chr10 | 89711798 | 89712045 | 0.367 | PTEN |
| chr10 | 89711867 | 89712069 | 0.399 | PTEN |
| chr10 | 89711880 | 89712129 | 0.348 | PTEN |
| chr10 | 89717531 | 89717770 | 0.404 | PTEN |
| chr10 | 89717558 | 89717802 | 0.408 | PTEN |
| chr10 | 89717558 | 89717831 | 0.394 | PTEN |
| chr10 | 89717571 | 89717820 | 0.400 | PTEN |
| chr10 | 89717602 | 89717831 | 0.413 | PTEN |
| chr10 | 89717603 | 89717769 | 0.455 | PTEN |
| chr10 | 89717627 | 89717872 | 0.362 | PTEN |
| chr10 | 89720757 | 89720967 | 0.336 | PTEN |
| chr10 | 89720757 | 89721005 | 0.329 | PTEN |
| chr10 | 89720767 | 89720968 | 0.337 | PTEN |
| chr10 | 89720775 | 89721018 | 0.324 | PTEN |
| chr10 | 89726371 | 89726571 | 0.289 | PTEN |
| chr10 | 89726794 | 89727038 | 0.310 | PTEN |
| chr10 | 89727021 | 89727240 | 0.282 | PTEN |
| chr10 | 89727261 | 89727519 | 0.317 | PTEN |
| chr10 | 89727756 | 89727916 | 0.404 | PTEN |
| chr10 | 89727978 | 89728203 | 0.389 | PTEN |
| chr10 | 89728310 | 89728512 | 0.389 | PTEN |
| chr10 | 89729027 | 89729257 | 0.251 | PTEN |
| chr10 | 89729816 | 89729967 | 0.336 | PTEN |
| chr10 | 89730284 | 89730433 | 0.307 | PTEN |
| chr10 | 89731453 | 89731610 | 0.329 | PTEN |
| chr11 | 22644366 | 22644570 | 0.273 | FANCF |
| chr11 | 22644511 | 22644731 | 0.344 | FANCF |
| chr11 | 22644738 | 22644938 | 0.318 | FANCF |
| chr11 | 22645645 | 22645808 | 0.348 | FANCF |
| chr11 | 22645808 | 22646060 | 0.352 | FANCF |
| chr11 | 22646058 | 22646268 | 0.322 | FANCF |
| chr11 | 22646388 | 22646588 | 0.532 | FANCF |
| chr11 | 22646657 | 22646927 | 0.598 | FANCF |
| chr11 | 22646959 | 22647229 | 0.657 | FANCF |
| chr11 | 22647378 | 22647578 | 0.478 | FANCF |
| chr11 | 47236728 | 47236949 | 0.635 | DDB2 |
| chr11 | 47237804 | 47238058 | 0.537 | DDB2 |
| chr11 | 47238291 | 47238491 | 0.483 | DDB2 |
| chr11 | 47254333 | 47254547 | 0.470 | DDB2 |
| chr11 | 47256241 | 47256494 | 0.547 | DDB2 |
| chr11 | 47256782 | 47257010 | 0.594 | DDB2 |
| chr11 | 47259397 | 47259552 | 0.468 | DDB2 |
| chr11 | 47259555 | 47259796 | 0.492 | DDB2 |
| chr11 | 47260567 | 47260808 | 0.483 | DDB2 |
| chr11 | 61197603 | 61197830 | 0.588 | SDHAF2 |
| chr11 | 61205114 | 61205296 | 0.481 | SDHAF2 |
| chr11 | 61205433 | 61205588 | 0.404 | SDHAF2 |
| chr11 | 61213390 | 61213639 | 0.488 | SDHAF2 |
| chr11 | 61213676 | 61213931 | 0.508 | SDHAF2 |
| chr11 | 61213967 | 61214232 | 0.462 | SDHAF2 |
| chr11 | 64570946 | 64571196 | 0.478 | MEN1 |
| chr11 | 64571178 | 64571436 | 0.544 | MEN1 |
| chr11 | 64571465 | 64571704 | 0.546 | MEN1 |
| chr11 | 64571732 | 64571978 | 0.567 | MEN1 |
| chr11 | 64572483 | 64572713 | 0.619 | MEN1 |
| chr11 | 64573013 | 64573278 | 0.602 | MEN1 |
| chr11 | 64573641 | 64573871 | 0.593 | MEN1 |
| chr11 | 64574483 | 64574728 | 0.602 | MEN1 |
| chr11 | 64575094 | 64575345 | 0.544 | MEN1 |
| chr11 | 64575352 | 64575623 | 0.614 | MEN1 |
| chr11 | 64577138 | 64577410 | 0.634 | MEN1 |
| chr11 | 64577437 | 64577683 | 0.700 | MEN1 |
| chr11 | 64577902 | 64578171 | 0.681 | MEN1 |
| chr11 | 94150558 | 94150800 | 0.362 | MRE11A |
| chr11 | 94151042 | 94151242 | 0.373 | MRE11A |
| chr11 | 94151616 | 94151816 | 0.537 | MRE11A |
| chr11 | 94151903 | 94152103 | 0.343 | MRE11A |
| chr11 | 94152190 | 94152390 | 0.333 | MRE11A |
| chr11 | 94153182 | 94153395 | 0.308 | MRE11A |
| chr11 | 94168979 | 94169178 | 0.335 | MRE11A |
| chr11 | 94170337 | 94170576 | 0.300 | MRE11A |
| chr11 | 94178876 | 94179116 | 0.386 | MRE11A |
| chr11 | 94180384 | 94180615 | 0.500 | MRE11A |
| chr11 | 94189360 | 94189588 | 0.323 | MRE11A |
| chr11 | 94192639 | 94192838 | 0.370 | MRE11A |
| chr11 | 94193992 | 94194257 | 0.301 | MRE11A |
| chr11 | 94197223 | 94197451 | 0.349 | MRE11A |
| chr11 | 94200864 | 94201064 | 0.363 | MRE11A |
| chr11 | 94203635 | 94203874 | 0.404 | MRE11A |
| chr11 | 94204708 | 94204908 | 0.368 | MRE11A |
| chr11 | 94209357 | 94209557 | 0.348 | MRE11A |
| chr11 | 94211862 | 94212106 | 0.359 | MRE11A |
| chr11 | 94212728 | 94212928 | 0.378 | MRE11A |
| chr11 | 94219015 | 94219215 | 0.348 | MRE11A |
| chr11 | 94219225 | 94219425 | 0.264 | MRE11A |
| chr11 | 94223880 | 94224120 | 0.344 | MRE11A |
| chr11 | 94223898 | 94224142 | 0.327 | MRE11A |
| chr11 | 94225885 | 94226125 | 0.394 | MRE11A |
| chr11 | 108093593 | 108093813 | 0.615 | ATM |
| chr11 | 108093873 | 108094073 | 0.617 | ATM |
| chr11 | 108098331 | 108098581 | 0.335 | ATM |
| chr11 | 108098372 | 108098572 | 0.333 | ATM |
| chr11 | 108098382 | 108098631 | 0.316 | ATM |
| chr11 | 108098397 | 108098626 | 0.309 | ATM |
| chr11 | 108098399 | 108098628 | 0.304 | ATM |
| chr11 | 108099818 | 108100062 | 0.327 | ATM |
| chr11 | 108106395 | 108106596 | 0.356 | ATM |
| chr11 | 108114723 | 108115004 | 0.333 | ATM |
| chr11 | 108114777 | 108115004 | 0.329 | ATM |
| chr11 | 108115492 | 108115736 | 0.351 | ATM |
| chr11 | 108117690 | 108117930 | 0.324 | ATM |
| chr11 | 108119692 | 108119907 | 0.343 | ATM |
| chr11 | 108119737 | 108119952 | 0.361 | ATM |
| chr11 | 108121367 | 108121602 | 0.377 | ATM |
| chr11 | 108121609 | 108121764 | 0.359 | ATM |
| chr11 | 108122506 | 108122706 | 0.363 | ATM |
| chr11 | 108122716 | 108122936 | 0.317 | ATM |
| chr11 | 108123498 | 108123718 | 0.321 | ATM |
| chr11 | 108124529 | 108124729 | 0.393 | ATM |
| chr11 | 108126954 | 108127154 | 0.363 | ATM |
| chr11 | 108128041 | 108128241 | 0.294 | ATM |
| chr11 | 108129523 | 108129756 | 0.316 | ATM |
| chr11 | 108137908 | 108138102 | 0.374 | ATM |
| chr11 | 108139112 | 108139322 | 0.374 | ATM |
| chr11 | 108139329 | 108139529 | 0.338 | ATM |
| chr11 | 108141794 | 108142040 | 0.324 | ATM |
| chr11 | 108142019 | 108142263 | 0.359 | ATM |
| chr11 | 108143141 | 108143341 | 0.323 | ATM |
| chr11 | 108143328 | 108143577 | 0.336 | ATM |
| chr11 | 108150264 | 108150498 | 0.328 | ATM |
| chr11 | 108151721 | 108151951 | 0.364 | ATM |
| chr11 | 108153427 | 108153675 | 0.285 | ATM |
| chr11 | 108153471 | 108153632 | 0.284 | ATM |
| chr11 | 108153471 | 108153677 | 0.275 | ATM |
| chr11 | 108153505 | 108153680 | 0.267 | ATM |
| chr11 | 108153510 | 108153680 | 0.269 | ATM |
| chr11 | 108154843 | 108155094 | 0.282 | ATM |
| chr11 | 108154858 | 108155070 | 0.291 | ATM |
| chr11 | 108154954 | 108155155 | 0.396 | ATM |
| chr11 | 108154962 | 108155211 | 0.388 | ATM |
| chr11 | 108157899 | 108158161 | 0.354 | ATM |
| chr11 | 108158376 | 108158580 | 0.337 | ATM |
| chr11 | 108159642 | 108159842 | 0.343 | ATM |
| chr11 | 108160273 | 108160473 | 0.299 | ATM |
| chr11 | 108163344 | 108163589 | 0.378 | ATM |
| chr11 | 108164044 | 108164225 | 0.324 | ATM |
| chr11 | 108164078 | 108164281 | 0.314 | ATM |
| chr11 | 108165595 | 108165795 | 0.358 | ATM |
| chr11 | 108167798 | 108168039 | 0.269 | ATM |
| chr11 | 108167811 | 108168065 | 0.267 | ATM |
| chr11 | 108170423 | 108170622 | 0.380 | ATM |
| chr11 | 108172321 | 108172561 | 0.340 | ATM |
| chr11 | 108173513 | 108173723 | 0.351 | ATM |
| chr11 | 108173723 | 108173923 | 0.313 | ATM |
| chr11 | 108175366 | 108175566 | 0.398 | ATM |
| chr11 | 108178629 | 108178814 | 0.360 | ATM |
| chr11 | 108179595 | 108179834 | 0.375 | ATM |
| chr11 | 108180801 | 108181050 | 0.320 | ATM |
| chr11 | 108180821 | 108181050 | 0.322 | ATM |
| chr11 | 108180821 | 108181066 | 0.313 | ATM |
| chr11 | 108180871 | 108181071 | 0.333 | ATM |
| chr11 | 108183026 | 108183226 | 0.333 | ATM |
| chr11 | 108186520 | 108186768 | 0.357 | ATM |
| chr11 | 108186672 | 108186911 | 0.404 | ATM |
| chr11 | 108188080 | 108188262 | 0.399 | ATM |
| chr11 | 108190668 | 108190878 | 0.313 | ATM |
| chr11 | 108191937 | 108192186 | 0.392 | ATM |
| chr11 | 108195999 | 108196199 | 0.413 | ATM |
| chr11 | 108196116 | 108196316 | 0.388 | ATM |
| chr11 | 108196772 | 108196944 | 0.422 | ATM |
| chr11 | 108198304 | 108198504 | 0.398 | ATM |
| chr11 | 108199732 | 108199932 | 0.373 | ATM |
| chr11 | 108200863 | 108201105 | 0.374 | ATM |
| chr11 | 108201993 | 108202237 | 0.318 | ATM |
| chr11 | 108202461 | 108202678 | 0.330 | ATM |
| chr11 | 108202483 | 108202676 | 0.309 | ATM |
| chr11 | 108202496 | 108202676 | 0.298 | ATM |
| chr11 | 108202530 | 108202730 | 0.308 | ATM |
| chr11 | 108203403 | 108203613 | 0.336 | ATM |
| chr11 | 108203427 | 108203679 | 0.344 | ATM |
| chr11 | 108203480 | 108203724 | 0.339 | ATM |
| chr11 | 108203524 | 108203768 | 0.339 | ATM |
| chr11 | 108203540 | 108203784 | 0.347 | ATM |
| chr11 | 108204436 | 108204676 | 0.407 | ATM |
| chr11 | 108205641 | 108205841 | 0.368 | ATM |
| chr11 | 108206452 | 108206651 | 0.400 | ATM |
| chr11 | 108213868 | 108214068 | 0.393 | ATM |
| chr11 | 108216481 | 108216700 | 0.355 | ATM |
| chr11 | 108217894 | 108218094 | 0.328 | ATM |
| chr11 | 108224395 | 108224595 | 0.403 | ATM |
| chr11 | 108225445 | 108225655 | 0.346 | ATM |
| chr11 | 108235722 | 108235932 | 0.365 | ATM |
| chr11 | 108235932 | 108236132 | 0.383 | ATM |
| chr11 | 108235986 | 108236232 | 0.433 | ATM |
| chr11 | 108236004 | 108236249 | 0.431 | ATM |
| chr11 | 108236050 | 108236290 | 0.415 | ATM |
| chr11 | 108236051 | 108236251 | 0.438 | ATM |
| chr11 | 108236071 | 108236273 | 0.438 | ATM |
| chr11 | 108238313 | 108238513 | 0.373 | ATM |
| chr11 | 111957513 | 111957759 | 0.591 | SDHD |
| chr11 | 111959529 | 111959746 | 0.486 | SDHD |
| chr11 | 111965449 | 111965740 | 0.414 | SDHD |
| chr11 | 111965464 | 111965754 | 0.416 | SDHD |
| chr12 | 25378488 | 25378688 | 0.353 | KRAS |
| chr12 | 25378503 | 25378751 | 0.341 | KRAS |
| chr12 | 25378546 | 25378778 | 0.352 | KRAS |
| chr12 | 25378554 | 25378783 | 0.348 | KRAS |
| chr12 | 25380153 | 25380359 | 0.411 | KRAS |
| chr12 | 25380166 | 25380337 | 0.407 | KRAS |
| chr12 | 25380167 | 25380326 | 0.406 | KRAS |
| chr12 | 25380167 | 25380359 | 0.420 | KRAS |
| chr12 | 25398080 | 25398329 | 0.360 | KRAS |
| chr12 | 25398145 | 25398394 | 0.348 | KRAS |
| chr12 | 25398153 | 25398397 | 0.351 | KRAS |
| chr12 | 25398159 | 25398408 | 0.356 | KRAS |
| chr12 | 25398186 | 25398433 | 0.347 | KRAS |
| chr12 | 56478765 | 56478994 | 0.552 | ERBB3 |
| chr12 | 56478781 | 56479029 | 0.558 | ERBB3 |
| chr12 | 56478784 | 56479029 | 0.561 | ERBB3 |
| chr12 | 56478807 | 56479047 | 0.560 | ERBB3 |
| chr12 | 56481533 | 56481774 | 0.541 | ERBB3 |
| chr12 | 56481559 | 56481798 | 0.521 | ERBB3 |
| chr12 | 56481594 | 56481833 | 0.546 | ERBB3 |
| chr12 | 56481628 | 56481942 | 0.530 | ERBB3 |
| chr12 | 56481740 | 56481979 | 0.517 | ERBB3 |
| chr12 | 56481773 | 56482020 | 0.476 | ERBB3 |
| chr12 | 56481807 | 56482048 | 0.467 | ERBB3 |
| chr12 | 56482218 | 56482457 | 0.513 | ERBB3 |
| chr12 | 56482252 | 56482491 | 0.496 | ERBB3 |
| chr12 | 56482278 | 56482521 | 0.504 | ERBB3 |
| chr12 | 56482331 | 56482580 | 0.528 | ERBB3 |
| chr12 | 56486559 | 56486791 | 0.502 | ERBB3 |
| chr12 | 56486561 | 56486791 | 0.502 | ERBB3 |
| chr12 | 56486566 | 56486813 | 0.508 | ERBB3 |
| chr12 | 56486569 | 56486818 | 0.508 | ERBB3 |
| chr12 | 56490766 | 56491006 | 0.523 | ERBB3 |
| chr12 | 56490766 | 56491013 | 0.520 | ERBB3 |
| chr12 | 56490773 | 56491013 | 0.519 | ERBB3 |
| chr12 | 56490777 | 56491011 | 0.523 | ERBB3 |
| chr12 | 56491580 | 56491801 | 0.554 | ERBB3 |
| chr12 | 56491592 | 56491801 | 0.557 | ERBB3 |
| chr12 | 56491596 | 56491799 | 0.559 | ERBB3 |
| chr12 | 56491596 | 56491801 | 0.558 | ERBB3 |
| chr12 | 58141882 | 58142151 | 0.396 | CDK4 |
| chr12 | 58142160 | 58142391 | 0.474 | CDK4 |
| chr12 | 58142983 | 58143287 | 0.570 | CDK4 |
| chr12 | 58142985 | 58143287 | 0.568 | CDK4 |
| chr12 | 58144413 | 58144686 | 0.478 | CDK4 |
| chr12 | 58144452 | 58144687 | 0.479 | CDK4 |
| chr12 | 58144692 | 58144932 | 0.490 | CDK4 |
| chr12 | 58144696 | 58144939 | 0.492 | CDK4 |
| chr12 | 58144957 | 58145214 | 0.523 | CDK4 |
| chr12 | 58145023 | 58145294 | 0.515 | CDK4 |
| chr12 | 58145309 | 58145580 | 0.544 | CDK4 |
| chr12 | 58145326 | 58145580 | 0.541 | CDK4 |
| chr12 | 58145924 | 58146140 | 0.673 | CDK4 |
| chr12 | 133200375 | 133200607 | 0.541 | POLE |
| chr12 | 133200794 | 133201005 | 0.604 | POLE |
| chr12 | 133200978 | 133201217 | 0.608 | POLE |
| chr12 | 133201185 | 133201425 | 0.610 | POLE |
| chr12 | 133201428 | 133201670 | 0.654 | POLE |
| chr12 | 133202187 | 133202429 | 0.621 | POLE |
| chr12 | 133202217 | 133202436 | 0.605 | POLE |
| chr12 | 133202217 | 133202437 | 0.606 | POLE |
| chr12 | 133202218 | 133202437 | 0.605 | POLE |
| chr12 | 133202355 | 133202555 | 0.622 | POLE |
| chr12 | 133202648 | 133202868 | 0.606 | POLE |
| chr12 | 133208842 | 133209042 | 0.517 | POLE |
| chr12 | 133209059 | 133209259 | 0.577 | POLE |
| chr12 | 133209191 | 133209391 | 0.647 | POLE |
| chr12 | 133210529 | 133210806 | 0.590 | POLE |
| chr12 | 133212413 | 133212654 | 0.488 | POLE |
| chr12 | 133214538 | 133214738 | 0.557 | POLE |
| chr12 | 133215637 | 133215885 | 0.582 | POLE |
| chr12 | 133218204 | 133218451 | 0.597 | POLE |
| chr12 | 133218707 | 133218953 | 0.579 | POLE |
| chr12 | 133219069 | 133219298 | 0.574 | POLE |
| chr12 | 133219399 | 133219599 | 0.617 | POLE |
| chr12 | 133219778 | 133220012 | 0.591 | POLE |
| chr12 | 133219996 | 133220226 | 0.606 | POLE |
| chr12 | 133220308 | 133220545 | 0.571 | POLE |
| chr12 | 133225529 | 133225773 | 0.612 | POLE |
| chr12 | 133225887 | 133226117 | 0.645 | POLE |
| chr12 | 133226161 | 133226408 | 0.613 | POLE |
| chr12 | 133233672 | 133233909 | 0.550 | POLE |
| chr12 | 133233774 | 133234003 | 0.557 | POLE |
| chr12 | 133234350 | 133234550 | 0.458 | POLE |
| chr12 | 133235953 | 133236173 | 0.534 | POLE |
| chr12 | 133237539 | 133237827 | 0.543 | POLE |
| chr12 | 133237547 | 133237781 | 0.519 | POLE |
| chr12 | 133238036 | 133238236 | 0.468 | POLE |
| chr12 | 133240495 | 133240681 | 0.545 | POLE |
| chr12 | 133241011 | 133241211 | 0.587 | POLE |
| chr12 | 133241805 | 133242034 | 0.600 | POLE |
| chr12 | 133244016 | 133244246 | 0.563 | POLE |
| chr12 | 133244860 | 133245070 | 0.578 | POLE |
| chr12 | 133245148 | 133245397 | 0.616 | POLE |
| chr12 | 133245378 | 133245623 | 0.537 | POLE |
| chr12 | 133248734 | 133248981 | 0.556 | POLE |
| chr12 | 133249169 | 133249404 | 0.568 | POLE |
| chr12 | 133249662 | 133249902 | 0.539 | POLE |
| chr12 | 133250240 | 133250445 | 0.549 | POLE |
| chr12 | 133250282 | 133250482 | 0.517 | POLE |
| chr12 | 133251897 | 133252126 | 0.600 | POLE |
| chr12 | 133252231 | 133252480 | 0.512 | POLE |
| chr12 | 133252591 | 133252826 | 0.470 | POLE |
| chr12 | 133253058 | 133253290 | 0.502 | POLE |
| chr12 | 133253869 | 133254099 | 0.429 | POLE |
| chr12 | 133254080 | 133254312 | 0.502 | POLE |
| chr12 | 133256057 | 133256299 | 0.494 | POLE |
| chr12 | 133256546 | 133256806 | 0.441 | POLE |
| chr12 | 133256751 | 133257006 | 0.469 | POLE |
| chr12 | 133257134 | 133257364 | 0.429 | POLE |
| chr12 | 133257609 | 133257851 | 0.523 | POLE |
| chr13 | 28592527 | 28592791 | 0.434 | FLT3 |
| chr13 | 32889625 | 32889857 | 0.631 | BRCA2 |
| chr13 | 32889901 | 32890111 | 0.583 | BRCA2 |
| chr13 | 32890514 | 32890741 | 0.346 | BRCA2 |
| chr13 | 32893139 | 32893383 | 0.339 | BRCA2 |
| chr13 | 32900135 | 32900368 | 0.252 | BRCA2 |
| chr13 | 32900239 | 32900484 | 0.305 | BRCA2 |
| chr13 | 32900514 | 32900762 | 0.373 | BRCA2 |
| chr13 | 32903445 | 32903674 | 0.278 | BRCA2 |
| chr13 | 32904938 | 32905182 | 0.343 | BRCA2 |
| chr13 | 32905002 | 32905201 | 0.315 | BRCA2 |
| chr13 | 32905048 | 32905165 | 0.339 | BRCA2 |
| chr13 | 32905048 | 32905168 | 0.347 | BRCA2 |
| chr13 | 32905048 | 32905170 | 0.341 | BRCA2 |
| chr13 | 32905049 | 32905165 | 0.342 | BRCA2 |
| chr13 | 32906224 | 32906468 | 0.302 | BRCA2 |
| chr13 | 32906406 | 32906650 | 0.310 | BRCA2 |
| chr13 | 32906408 | 32906628 | 0.317 | BRCA2 |
| chr13 | 32906426 | 32906673 | 0.306 | BRCA2 |
| chr13 | 32906464 | 32906663 | 0.305 | BRCA2 |
| chr13 | 32906520 | 32906768 | 0.317 | BRCA2 |
| chr13 | 32906575 | 32906818 | 0.361 | BRCA2 |
| chr13 | 32906606 | 32906846 | 0.378 | BRCA2 |
| chr13 | 32906668 | 32906912 | 0.388 | BRCA2 |
| chr13 | 32906748 | 32906987 | 0.388 | BRCA2 |
| chr13 | 32906815 | 32907062 | 0.363 | BRCA2 |
| chr13 | 32906856 | 32907103 | 0.367 | BRCA2 |
| chr13 | 32906893 | 32907106 | 0.383 | BRCA2 |
| chr13 | 32906938 | 32907183 | 0.378 | BRCA2 |
| chr13 | 32907059 | 32907264 | 0.374 | BRCA2 |
| chr13 | 32907059 | 32907307 | 0.378 | BRCA2 |
| chr13 | 32907288 | 32907533 | 0.350 | BRCA2 |
| chr13 | 32910416 | 32910655 | 0.354 | BRCA2 |
| chr13 | 32910596 | 32910835 | 0.388 | BRCA2 |
| chr13 | 32910778 | 32911027 | 0.340 | BRCA2 |
| chr13 | 32910967 | 32911215 | 0.317 | BRCA2 |
| chr13 | 32910988 | 32911187 | 0.335 | BRCA2 |
| chr13 | 32911008 | 32911252 | 0.331 | BRCA2 |
| chr13 | 32911035 | 32911252 | 0.321 | BRCA2 |
| chr13 | 32911045 | 32911295 | 0.331 | BRCA2 |
| chr13 | 32911167 | 32911415 | 0.341 | BRCA2 |
| chr13 | 32911340 | 32911588 | 0.333 | BRCA2 |
| chr13 | 32911594 | 32911838 | 0.322 | BRCA2 |
| chr13 | 32911841 | 32912085 | 0.384 | BRCA2 |
| chr13 | 32912080 | 32912319 | 0.342 | BRCA2 |
| chr13 | 32912267 | 32912511 | 0.265 | BRCA2 |
| chr13 | 32912502 | 32912746 | 0.331 | BRCA2 |
| chr13 | 32912749 | 32912986 | 0.307 | BRCA2 |
| chr13 | 32912979 | 32913218 | 0.404 | BRCA2 |
| chr13 | 32913217 | 32913460 | 0.336 | BRCA2 |
| chr13 | 32913444 | 32913691 | 0.323 | BRCA2 |
| chr13 | 32913682 | 32913927 | 0.321 | BRCA2 |
| chr13 | 32913944 | 32914192 | 0.329 | BRCA2 |
| chr13 | 32914208 | 32914455 | 0.347 | BRCA2 |
| chr13 | 32914462 | 32914709 | 0.343 | BRCA2 |
| chr13 | 32914691 | 32914936 | 0.329 | BRCA2 |
| chr13 | 32914776 | 32915021 | 0.333 | BRCA2 |
| chr13 | 32914895 | 32915115 | 0.326 | BRCA2 |
| chr13 | 32914896 | 32915115 | 0.327 | BRCA2 |
| chr13 | 32914906 | 32915155 | 0.328 | BRCA2 |
| chr13 | 32915087 | 32915334 | 0.355 | BRCA2 |
| chr13 | 32915144 | 32915384 | 0.357 | BRCA2 |
| chr13 | 32918540 | 32918787 | 0.258 | BRCA2 |
| chr13 | 32920834 | 32921033 | 0.295 | BRCA2 |
| chr13 | 32928970 | 32929189 | 0.345 | BRCA2 |
| chr13 | 32928972 | 32929201 | 0.357 | BRCA2 |
| chr13 | 32928992 | 32929236 | 0.351 | BRCA2 |
| chr13 | 32928996 | 32929196 | 0.358 | BRCA2 |
| chr13 | 32928996 | 32929208 | 0.357 | BRCA2 |
| chr13 | 32929176 | 32929423 | 0.339 | BRCA2 |
| chr13 | 32929177 | 32929426 | 0.344 | BRCA2 |
| chr13 | 32929220 | 32929467 | 0.323 | BRCA2 |
| chr13 | 32929274 | 32929479 | 0.335 | BRCA2 |
| chr13 | 32929297 | 32929498 | 0.322 | BRCA2 |
| chr13 | 32930589 | 32930789 | 0.448 | BRCA2 |
| chr13 | 32931650 | 32931879 | 0.257 | BRCA2 |
| chr13 | 32931817 | 32932017 | 0.318 | BRCA2 |
| chr13 | 32932034 | 32932234 | 0.313 | BRCA2 |
| chr13 | 32936641 | 32936885 | 0.384 | BRCA2 |
| chr13 | 32937319 | 32937563 | 0.384 | BRCA2 |
| chr13 | 32937529 | 32937773 | 0.376 | BRCA2 |
| chr13 | 32944444 | 32944688 | 0.359 | BRCA2 |
| chr13 | 32945080 | 32945249 | 0.359 | BRCA2 |
| chr13 | 32950820 | 32951019 | 0.440 | BRCA2 |
| chr13 | 32953333 | 32953533 | 0.348 | BRCA2 |
| chr13 | 32953442 | 32953686 | 0.363 | BRCA2 |
| chr13 | 32953840 | 32954084 | 0.327 | BRCA2 |
| chr13 | 32954054 | 32954299 | 0.346 | BRCA2 |
| chr13 | 32954054 | 32954300 | 0.344 | BRCA2 |
| chr13 | 32968741 | 32968971 | 0.359 | BRCA2 |
| chr13 | 32968820 | 32969069 | 0.384 | BRCA2 |
| chr13 | 32970989 | 32971236 | 0.379 | BRCA2 |
| chr13 | 32971106 | 32971335 | 0.348 | BRCA2 |
| chr13 | 32972257 | 32972489 | 0.373 | BRCA2 |
| chr13 | 32972463 | 32972703 | 0.390 | BRCA2 |
| chr13 | 32972600 | 32972845 | 0.378 | BRCA2 |
| chr13 | 32972664 | 32972864 | 0.418 | BRCA2 |
| chr13 | 32972671 | 32972922 | 0.397 | BRCA2 |
| chr13 | 32972708 | 32972954 | 0.389 | BRCA2 |
| chr13 | 32973392 | 32973641 | 0.336 | BRCA2 |
| chr13 | 32973613 | 32973807 | 0.267 | BRCA2 |
| chr13 | 48916668 | 48916868 | 0.318 | RB1 |
| chr13 | 48919151 | 48919351 | 0.284 | RB1 |
| chr13 | 48921945 | 48922145 | 0.308 | RB1 |
| chr13 | 48923001 | 48923221 | 0.258 | RB1 |
| chr13 | 48936897 | 48937117 | 0.330 | RB1 |
| chr13 | 48939068 | 48939268 | 0.318 | RB1 |
| chr13 | 48941560 | 48941760 | 0.318 | RB1 |
| chr13 | 48947429 | 48947629 | 0.323 | RB1 |
| chr13 | 48951017 | 48951237 | 0.348 | RB1 |
| chr13 | 48954364 | 48954564 | 0.328 | RB1 |
| chr13 | 48955326 | 48955526 | 0.303 | RB1 |
| chr13 | 48955536 | 48955736 | 0.338 | RB1 |
| chr13 | 49027033 | 49027233 | 0.333 | RB1 |
| chr13 | 49030212 | 49030472 | 0.364 | RB1 |
| chr13 | 49033792 | 49033992 | 0.428 | RB1 |
| chr13 | 49037854 | 49038084 | 0.299 | RB1 |
| chr13 | 49039190 | 49039390 | 0.378 | RB1 |
| chr13 | 49039267 | 49039467 | 0.383 | RB1 |
| chr13 | 49047356 | 49047616 | 0.287 | RB1 |
| chr13 | 49050783 | 49050983 | 0.373 | RB1 |
| chr13 | 49051484 | 49051724 | 0.299 | RB1 |
| chr13 | 49054133 | 49054333 | 0.413 | RB1 |
| chr13 | 49054700 | 49054910 | 0.251 | RB1 |
| chr13 | 49055078 | 49055278 | 0.323 | RB1 |
| chr13 | 49055456 | 49055656 | 0.343 | RB1 |
| chr13 | 49055834 | 49056034 | 0.254 | RB1 |
| chr13 | 103498123 | 103498440 | 0.629 | ERCC5 |
| chr13 | 103498155 | 103498432 | 0.640 | ERCC5 |
| chr13 | 103498190 | 103498390 | 0.642 | ERCC5 |
| chr13 | 103498192 | 103498440 | 0.631 | ERCC5 |
| chr13 | 103498192 | 103498453 | 0.626 | ERCC5 |
| chr13 | 103498470 | 103498701 | 0.608 | ERCC5 |
| chr13 | 103498494 | 103498713 | 0.618 | ERCC5 |
| chr13 | 103498494 | 103498717 | 0.621 | ERCC5 |
| chr13 | 103498574 | 103498757 | 0.641 | ERCC5 |
| chr13 | 103504312 | 103504529 | 0.349 | ERCC5 |
| chr13 | 103504364 | 103504578 | 0.372 | ERCC5 |
| chr13 | 103506028 | 103506230 | 0.409 | ERCC5 |
| chr13 | 103506526 | 103506726 | 0.463 | ERCC5 |
| chr13 | 103508374 | 103508544 | 0.275 | ERCC5 |
| chr13 | 103510603 | 103510803 | 0.393 | ERCC5 |
| chr13 | 103513836 | 103514056 | 0.407 | ERCC5 |
| chr13 | 103514321 | 103514572 | 0.433 | ERCC5 |
| chr13 | 103514574 | 103514792 | 0.534 | ERCC5 |
| chr13 | 103514801 | 103515020 | 0.482 | ERCC5 |
| chr13 | 103515021 | 103515221 | 0.428 | ERCC5 |
| chr13 | 103515235 | 103515435 | 0.433 | ERCC5 |
| chr13 | 103517984 | 103518184 | 0.478 | ERCC5 |
| chr13 | 103518194 | 103518404 | 0.412 | ERCC5 |
| chr13 | 103518516 | 103518726 | 0.370 | ERCC5 |
| chr13 | 103519057 | 103519257 | 0.368 | ERCC5 |
| chr13 | 103520541 | 103520748 | 0.370 | ERCC5 |
| chr13 | 103524493 | 103524693 | 0.413 | ERCC5 |
| chr13 | 103524585 | 103524802 | 0.445 | ERCC5 |
| chr13 | 103525496 | 103525696 | 0.368 | ERCC5 |
| chr13 | 103527656 | 103527856 | 0.408 | ERCC5 |
| chr13 | 103527863 | 103528063 | 0.408 | ERCC5 |
| chr13 | 103528070 | 103528300 | 0.437 | ERCC5 |
| chr14 | 38060515 | 38060750 | 0.589 | FOXA1 |
| chr14 | 38060564 | 38060810 | 0.591 | FOXA1 |
| chr14 | 38060574 | 38060818 | 0.592 | FOXA1 |
| chr14 | 38060586 | 38060833 | 0.601 | FOXA1 |
| chr14 | 45605103 | 45605333 | 0.558 | FANCM |
| chr14 | 45605305 | 45605553 | 0.610 | FANCM |
| chr14 | 45605573 | 45605799 | 0.511 | FANCM |
| chr14 | 45606233 | 45606433 | 0.398 | FANCM |
| chr14 | 45618024 | 45618224 | 0.363 | FANCM |
| chr14 | 45620541 | 45620741 | 0.333 | FANCM |
| chr14 | 45623034 | 45623234 | 0.348 | FANCM |
| chr14 | 45628297 | 45628497 | 0.408 | FANCM |
| chr14 | 45633510 | 45633710 | 0.373 | FANCM |
| chr14 | 45633720 | 45633920 | 0.383 | FANCM |
| chr14 | 45636166 | 45636386 | 0.376 | FANCM |
| chr14 | 45639788 | 45640013 | 0.341 | FANCM |
| chr14 | 45642242 | 45642442 | 0.403 | FANCM |
| chr14 | 45644281 | 45644486 | 0.340 | FANCM |
| chr14 | 45644653 | 45644863 | 0.313 | FANCM |
| chr14 | 45644862 | 45645099 | 0.382 | FANCM |
| chr14 | 45645143 | 45645342 | 0.350 | FANCM |
| chr14 | 45645413 | 45645613 | 0.378 | FANCM |
| chr14 | 45645666 | 45645955 | 0.331 | FANCM |
| chr14 | 45645983 | 45646183 | 0.303 | FANCM |
| chr14 | 45650718 | 45650970 | 0.332 | FANCM |
| chr14 | 45650772 | 45650971 | 0.320 | FANCM |
| chr14 | 45652886 | 45653086 | 0.338 | FANCM |
| chr14 | 45654389 | 45654589 | 0.303 | FANCM |
| chr14 | 45656912 | 45657134 | 0.269 | FANCM |
| chr14 | 45658004 | 45658204 | 0.333 | FANCM |
| chr14 | 45658183 | 45658427 | 0.371 | FANCM |
| chr14 | 45658417 | 45658644 | 0.368 | FANCM |
| chr14 | 45665608 | 45665811 | 0.348 | FANCM |
| chr14 | 45667934 | 45668145 | 0.358 | FANCM |
| chr14 | 45668983 | 45669204 | 0.320 | FANCM |
| chr14 | 45669474 | 45669678 | 0.346 | FANCM |
| chr14 | 95557315 | 95557515 | 0.313 | DICER1 |
| chr14 | 95557526 | 95557726 | 0.463 | DICER1 |
| chr14 | 95559982 | 95560182 | 0.542 | DICER1 |
| chr14 | 95560204 | 95560434 | 0.494 | DICER1 |
| chr14 | 95560444 | 95560644 | 0.398 | DICER1 |
| chr14 | 95562161 | 95562361 | 0.368 | DICER1 |
| chr14 | 95562361 | 95562592 | 0.530 | DICER1 |
| chr14 | 95562601 | 95562801 | 0.438 | DICER1 |
| chr14 | 95562666 | 95562891 | 0.398 | DICER1 |
| chr14 | 95566129 | 95566335 | 0.406 | DICER1 |
| chr14 | 95569682 | 95569882 | 0.468 | DICER1 |
| chr14 | 95569994 | 95570194 | 0.403 | DICER1 |
| chr14 | 95570228 | 95570428 | 0.418 | DICER1 |
| chr14 | 95571340 | 95571540 | 0.488 | DICER1 |
| chr14 | 95571974 | 95572174 | 0.348 | DICER1 |
| chr14 | 95572314 | 95572514 | 0.428 | DICER1 |
| chr14 | 95572524 | 95572764 | 0.249 | DICER1 |
| chr14 | 95573959 | 95574159 | 0.338 | DICER1 |
| chr14 | 95574168 | 95574368 | 0.348 | DICER1 |
| chr14 | 95574657 | 95574857 | 0.443 | DICER1 |
| chr14 | 95577599 | 95577799 | 0.433 | DICER1 |
| chr14 | 95578391 | 95578591 | 0.358 | DICER1 |
| chr14 | 95579340 | 95579540 | 0.358 | DICER1 |
| chr14 | 95581926 | 95582126 | 0.433 | DICER1 |
| chr14 | 95582756 | 95582956 | 0.358 | DICER1 |
| chr14 | 95582966 | 95583166 | 0.348 | DICER1 |
| chr14 | 95583900 | 95584100 | 0.378 | DICER1 |
| chr14 | 95590532 | 95590732 | 0.358 | DICER1 |
| chr14 | 95590767 | 95590967 | 0.393 | DICER1 |
| chr14 | 95592822 | 95593064 | 0.305 | DICER1 |
| chr14 | 95595734 | 95595934 | 0.358 | DICER1 |
| chr14 | 95596337 | 95596537 | 0.343 | DICER1 |
| chr14 | 95598778 | 95598978 | 0.383 | DICER1 |
| chr14 | 95598988 | 95599188 | 0.308 | DICER1 |
| chr14 | 95599591 | 95599801 | 0.436 | DICER1 |
| chr14 | 95623722 | 95623973 | 0.750 | DICER1 |
| chr14 | 105246388 | 105246637 | 0.596 | AKT1 |
| chr14 | 105246483 | 105246730 | 0.617 | AKT1 |
| chr14 | 105246501 | 105246707 | 0.633 | AKT1 |
| chr14 | 105246501 | 105246745 | 0.633 | AKT1 |
| chr15 | 32968921 | 32969121 | 0.303 | GREM1 |
| chr15 | 32976883 | 32977083 | 0.393 | GREM1 |
| chr15 | 32984845 | 32985055 | 0.469 | GREM1 |
| chr15 | 32988826 | 32989036 | 0.370 | SCG5 |
| chr15 | 33000769 | 33000969 | 0.512 | GREM1 |
| chr15 | 33022952 | 33023162 | 0.654 | GREM1 |
| chr15 | 33022952 | 33023205 | 0.638 | GREM1 |
| chr15 | 33023018 | 33023279 | 0.626 | GREM1 |
| chr15 | 33023148 | 33023435 | 0.521 | GREM1 |
| chr15 | 33023151 | 33023435 | 0.519 | GREM1 |
| chr15 | 33023205 | 33023450 | 0.504 | GREM1 |
| chr15 | 33023686 | 33023886 | 0.517 | GREM1 |
| chr15 | 33024084 | 33024294 | 0.464 | GREM1 |
| chr15 | 33024482 | 33024682 | 0.398 | GREM1 |
| chr15 | 33026472 | 33026672 | 0.299 | GREM1 |
| chr15 | 66727327 | 66727566 | 0.546 | MAP2K1 |
| chr15 | 66727339 | 66727587 | 0.550 | MAP2K1 |
| chr15 | 66727339 | 66727588 | 0.548 | MAP2K1 |
| chr15 | 66727359 | 66727598 | 0.542 | MAP2K1 |
| chr15 | 66729024 | 66729277 | 0.496 | MAP2K1 |
| chr15 | 66729065 | 66729264 | 0.515 | MAP2K1 |
| chr15 | 66774016 | 66774220 | 0.527 | MAP2K1 |
| chr15 | 66774016 | 66774260 | 0.510 | MAP2K1 |
| chr15 | 66774048 | 66774260 | 0.516 | MAP2K1 |
| chr15 | 66774052 | 66774260 | 0.517 | MAP2K1 |
| chr15 | 66777305 | 66777541 | 0.591 | MAP2K1 |
| chr15 | 66777336 | 66777568 | 0.597 | MAP2K1 |
| chr15 | 66777336 | 66777570 | 0.600 | MAP2K1 |
| chr15 | 66777338 | 66777568 | 0.597 | MAP2K1 |
| chr15 | 89787224 | 89787484 | 0.651 | FANCI |
| chr15 | 89790756 | 89790956 | 0.368 | FANCI |
| chr15 | 89801818 | 89802048 | 0.403 | FANCI |
| chr15 | 89803854 | 89804054 | 0.403 | FANCI |
| chr15 | 89804662 | 89804884 | 0.372 | FANCI |
| chr15 | 89804949 | 89805159 | 0.365 | FANCI |
| chr15 | 89806635 | 89806874 | 0.371 | FANCI |
| chr15 | 89807040 | 89807240 | 0.403 | FANCI |
| chr15 | 89807671 | 89807891 | 0.344 | FANCI |
| chr15 | 89811673 | 89811922 | 0.364 | FANCI |
| chr15 | 89817473 | 89817702 | 0.370 | FANCI |
| chr15 | 89819937 | 89820137 | 0.418 | FANCI |
| chr15 | 89821824 | 89822080 | 0.358 | FANCI |
| chr15 | 89824311 | 89824511 | 0.333 | FANCI |
| chr15 | 89824830 | 89825066 | 0.359 | FANCI |
| chr15 | 89828191 | 89828429 | 0.439 | FANCI |
| chr15 | 89833373 | 89833592 | 0.368 | FANCI |
| chr15 | 89834739 | 89834949 | 0.365 | FANCI |
| chr15 | 89835684 | 89835936 | 0.336 | FANCI |
| chr15 | 89836099 | 89836321 | 0.327 | FANCI |
| chr15 | 89837021 | 89837221 | 0.358 | FANCI |
| chr15 | 89838207 | 89838356 | 0.480 | FANCI |
| chr15 | 89843116 | 89843345 | 0.417 | FANCI |
| chr15 | 89843449 | 89843649 | 0.403 | FANCI |
| chr15 | 89844490 | 89844700 | 0.455 | FANCI |
| chr15 | 89846965 | 89847165 | 0.388 | FANCI |
| chr15 | 89848390 | 89848629 | 0.479 | FANCI |
| chr15 | 89848727 | 89848927 | 0.478 | FANCI |
| chr15 | 89849176 | 89849396 | 0.439 | FANCI |
| chr15 | 89850578 | 89850778 | 0.403 | FANCI |
| chr15 | 89850795 | 89850995 | 0.438 | FANCI |
| chr15 | 89857735 | 89857935 | 0.398 | FANCI |
| chr15 | 89858460 | 89858694 | 0.498 | FANCI |
| chr15 | 89859420 | 89859663 | 0.430 | FANCI |
| chr15 | 89859773 | 89859973 | 0.517 | FANCI |
| chr15 | 89859992 | 89860192 | 0.443 | FANCI |
| chr15 | 89860258 | 89860477 | 0.359 | FANCI |
| chr15 | 90631785 | 90632007 | 0.570 | IDH2 |
| chr15 | 90631814 | 90632043 | 0.548 | IDH2 |
| chr15 | 90631843 | 90632054 | 0.519 | IDH2 |
| chr16 | 3631168 | 3631388 | 0.443 | SLX4 |
| chr16 | 3632424 | 3632665 | 0.645 | SLX4 |
| chr16 | 3632693 | 3632893 | 0.597 | SLX4 |
| chr16 | 3633083 | 3633283 | 0.582 | SLX4 |
| chr16 | 3633339 | 3633565 | 0.590 | SLX4 |
| chr16 | 3634696 | 3634896 | 0.502 | SLX4 |
| chr16 | 3639002 | 3639202 | 0.617 | SLX4 |
| chr16 | 3639197 | 3639423 | 0.652 | SLX4 |
| chr16 | 3639505 | 3639754 | 0.632 | SLX4 |
| chr16 | 3639692 | 3639932 | 0.639 | SLX4 |
| chr16 | 3639911 | 3640159 | 0.530 | SLX4 |
| chr16 | 3640185 | 3640409 | 0.498 | SLX4 |
| chr16 | 3640478 | 3640721 | 0.607 | SLX4 |
| chr16 | 3640760 | 3641047 | 0.660 | SLX4 |
| chr16 | 3641072 | 3641272 | 0.468 | SLX4 |
| chr16 | 3642628 | 3642858 | 0.597 | SLX4 |
| chr16 | 3644537 | 3644773 | 0.608 | SLX4 |
| chr16 | 3645495 | 3645705 | 0.673 | SLX4 |
| chr16 | 3646003 | 3646244 | 0.698 | SLX4 |
| chr16 | 3646031 | 3646244 | 0.720 | SLX4 |
| chr16 | 3646124 | 3646405 | 0.699 | SLX4 |
| chr16 | 3646149 | 3646391 | 0.712 | SLX4 |
| chr16 | 3646157 | 3646403 | 0.704 | SLX4 |
| chr16 | 3647312 | 3647583 | 0.610 | SLX4 |
| chr16 | 3647601 | 3647800 | 0.540 | SLX4 |
| chr16 | 3647864 | 3648074 | 0.578 | SLX4 |
| chr16 | 3650971 | 3651251 | 0.530 | SLX4 |
| chr16 | 3652058 | 3652258 | 0.557 | SLX4 |
| chr16 | 3656390 | 3656622 | 0.506 | SLX4 |
| chr16 | 3656642 | 3656842 | 0.443 | SLX4 |
| chr16 | 3658480 | 3658725 | 0.528 | SLX4 |
| chr16 | 3658510 | 3658671 | 0.562 | SLX4 |
| chr16 | 3658784 | 3659014 | 0.455 | SLX4 |
| chr16 | 3659560 | 3659760 | 0.438 | SLX4 |
| chr16 | 14013935 | 14014175 | 0.651 | ERCC4 |
| chr16 | 14013966 | 14014166 | 0.662 | ERCC4 |
| chr16 | 14013970 | 14014170 | 0.662 | ERCC4 |
| chr16 | 14013971 | 14014170 | 0.665 | ERCC4 |
| chr16 | 14015823 | 14016023 | 0.373 | ERCC4 |
| chr16 | 14016033 | 14016243 | 0.355 | ERCC4 |
| chr16 | 14020466 | 14020710 | 0.351 | ERCC4 |
| chr16 | 14021912 | 14022151 | 0.338 | ERCC4 |
| chr16 | 14024532 | 14024732 | 0.373 | ERCC4 |
| chr16 | 14025949 | 14026157 | 0.344 | ERCC4 |
| chr16 | 14027949 | 14028149 | 0.348 | ERCC4 |
| chr16 | 14028984 | 14029224 | 0.415 | ERCC4 |
| chr16 | 14029445 | 14029685 | 0.452 | ERCC4 |
| chr16 | 14031514 | 14031714 | 0.373 | ERCC4 |
| chr16 | 14038575 | 14038734 | 0.494 | ERCC4 |
| chr16 | 14041470 | 14041670 | 0.468 | ERCC4 |
| chr16 | 14041655 | 14041919 | 0.509 | ERCC4 |
| chr16 | 14041900 | 14042126 | 0.498 | ERCC4 |
| chr16 | 14042120 | 14042369 | 0.404 | ERCC4 |
| chr16 | 14042584 | 14042854 | 0.424 | ERCC4 |
| chr16 | 14042844 | 14043078 | 0.336 | ERCC4 |
| chr16 | 14042902 | 14043146 | 0.347 | ERCC4 |
| chr16 | 14043430 | 14043679 | 0.284 | ERCC4 |
| chr16 | 14044214 | 14044436 | 0.332 | ERCC4 |
| chr16 | 14045149 | 14045380 | 0.276 | ERCC4 |
| chr16 | 14045389 | 14045634 | 0.427 | ERCC4 |
| chr16 | 14045727 | 14045927 | 0.328 | ERCC4 |
| chr16 | 14046154 | 14046384 | 0.303 | ERCC4 |
| chr16 | 23614570 | 23614827 | 0.310 | PALB2 |
| chr16 | 23614836 | 23615076 | 0.407 | PALB2 |
| chr16 | 23619110 | 23619349 | 0.479 | PALB2 |
| chr16 | 23625213 | 23625413 | 0.398 | PALB2 |
| chr16 | 23632741 | 23632891 | 0.391 | PALB2 |
| chr16 | 23634215 | 23634415 | 0.393 | PALB2 |
| chr16 | 23635260 | 23635465 | 0.374 | PALB2 |
| chr16 | 23637513 | 23637713 | 0.458 | PALB2 |
| chr16 | 23640440 | 23640682 | 0.370 | PALB2 |
| chr16 | 23640927 | 23641175 | 0.470 | PALB2 |
| chr16 | 23641292 | 23641502 | 0.431 | PALB2 |
| chr16 | 23641570 | 23641813 | 0.385 | PALB2 |
| chr16 | 23646217 | 23646416 | 0.470 | PALB2 |
| chr16 | 23646250 | 23646449 | 0.460 | PALB2 |
| chr16 | 23646427 | 23646636 | 0.390 | PALB2 |
| chr16 | 23646623 | 23646823 | 0.403 | PALB2 |
| chr16 | 23646780 | 23647008 | 0.345 | PALB2 |
| chr16 | 23646984 | 23647228 | 0.400 | PALB2 |
| chr16 | 23647144 | 23647434 | 0.385 | PALB2 |
| chr16 | 23647358 | 23647558 | 0.483 | PALB2 |
| chr16 | 23647369 | 23647610 | 0.467 | PALB2 |
| chr16 | 23649127 | 23649357 | 0.355 | PALB2 |
| chr16 | 23649265 | 23649465 | 0.358 | PALB2 |
| chr16 | 23652430 | 23652650 | 0.692 | PALB2 |
| chr16 | 23652647 | 23652887 | 0.647 | PALB2 |
| chr16 | 68772169 | 68772389 | 0.679 | CDH1 |
| chr16 | 68835529 | 68835729 | 0.463 | CDH1 |
| chr16 | 68835564 | 68835767 | 0.485 | CDH1 |
| chr16 | 68842243 | 68842453 | 0.469 | CDH1 |
| chr16 | 68842518 | 68842718 | 0.423 | CDH1 |
| chr16 | 68844017 | 68844227 | 0.512 | CDH1 |
| chr16 | 68845519 | 68845719 | 0.483 | CDH1 |
| chr16 | 68845706 | 68845945 | 0.517 | CDH1 |
| chr16 | 68845947 | 68846147 | 0.502 | CDH1 |
| chr16 | 68846157 | 68846377 | 0.416 | CDH1 |
| chr16 | 68847216 | 68847450 | 0.477 | CDH1 |
| chr16 | 68849385 | 68849585 | 0.488 | CDH1 |
| chr16 | 68849423 | 68849649 | 0.529 | CDH1 |
| chr16 | 68853169 | 68853348 | 0.522 | CDH1 |
| chr16 | 68855861 | 68856061 | 0.453 | CDH1 |
| chr16 | 68856071 | 68856271 | 0.488 | CDH1 |
| chr16 | 68856802 | 68857067 | 0.560 | CDH1 |
| chr16 | 68857287 | 68857494 | 0.481 | CDH1 |
| chr16 | 68862023 | 68862255 | 0.511 | CDH1 |
| chr16 | 68863473 | 68863683 | 0.488 | CDH1 |
| chr16 | 68867121 | 68867366 | 0.488 | CDH1 |
| chr16 | 68867369 | 68867599 | 0.476 | CDH1 |
| chr16 | 89804046 | 89804215 | 0.553 | FANCA |
| chr16 | 89804303 | 89804513 | 0.592 | FANCA |
| chr16 | 89804648 | 89804858 | 0.635 | FANCA |
| chr16 | 89804928 | 89805172 | 0.596 | FANCA |
| chr16 | 89805241 | 89805441 | 0.498 | FANCA |
| chr16 | 89805462 | 89805662 | 0.612 | FANCA |
| chr16 | 89805672 | 89805892 | 0.548 | FANCA |
| chr16 | 89806299 | 89806509 | 0.502 | FANCA |
| chr16 | 89807188 | 89807437 | 0.364 | FANCA |
| chr16 | 89809197 | 89809432 | 0.542 | FANCA |
| chr16 | 89811314 | 89811494 | 0.646 | FANCA |
| chr16 | 89812889 | 89813109 | 0.557 | FANCA |
| chr16 | 89813161 | 89813385 | 0.551 | FANCA |
| chr16 | 89814966 | 89815166 | 0.587 | FANCA |
| chr16 | 89816227 | 89816392 | 0.608 | FANCA |
| chr16 | 89818546 | 89818699 | 0.416 | FANCA |
| chr16 | 89825033 | 89825256 | 0.563 | FANCA |
| chr16 | 89825068 | 89825276 | 0.545 | FANCA |
| chr16 | 89828413 | 89828604 | 0.396 | FANCA |
| chr16 | 89831297 | 89831493 | 0.533 | FANCA |
| chr16 | 89833531 | 89833695 | 0.358 | FANCA |
| chr16 | 89836321 | 89836521 | 0.617 | FANCA |
| chr16 | 89836550 | 89836790 | 0.598 | FANCA |
| chr16 | 89836823 | 89837062 | 0.604 | FANCA |
| chr16 | 89836842 | 89837075 | 0.615 | FANCA |
| chr16 | 89836876 | 89837138 | 0.635 | FANCA |
| chr16 | 89836882 | 89837082 | 0.627 | FANCA |
| chr16 | 89836894 | 89837156 | 0.627 | FANCA |
| chr16 | 89838040 | 89838261 | 0.514 | FANCA |
| chr16 | 89839593 | 89839838 | 0.573 | FANCA |
| chr16 | 89842062 | 89842262 | 0.498 | FANCA |
| chr16 | 89845140 | 89845350 | 0.483 | FANCA |
| chr16 | 89845256 | 89845456 | 0.527 | FANCA |
| chr16 | 89846317 | 89846557 | 0.498 | FANCA |
| chr16 | 89849141 | 89849341 | 0.502 | FANCA |
| chr16 | 89849323 | 89849591 | 0.554 | FANCA |
| chr16 | 89857774 | 89858016 | 0.560 | FANCA |
| chr16 | 89858250 | 89858450 | 0.567 | FANCA |
| chr16 | 89858752 | 89858951 | 0.555 | FANCA |
| chr16 | 89862215 | 89862425 | 0.512 | FANCA |
| chr16 | 89865452 | 89865652 | 0.532 | FANCA |
| chr16 | 89865648 | 89865886 | 0.498 | FANCA |
| chr16 | 89865874 | 89866074 | 0.408 | FANCA |
| chr16 | 89869678 | 89869909 | 0.461 | FANCA |
| chr16 | 89871619 | 89871819 | 0.522 | FANCA |
| chr16 | 89874567 | 89874718 | 0.375 | FANCA |
| chr16 | 89877021 | 89877274 | 0.382 | FANCA |
| chr16 | 89877278 | 89877525 | 0.520 | FANCA |
| chr16 | 89880876 | 89881036 | 0.342 | FANCA |
| chr16 | 89882268 | 89882508 | 0.564 | FANCA |
| chr17 | 7572710 | 7572941 | 0.547 | TP53 |
| chr17 | 7572841 | 7573094 | 0.524 | TP53 |
| chr17 | 7573785 | 7574015 | 0.571 | TP53 |
| chr17 | 7573785 | 7574050 | 0.583 | TP53 |
| chr17 | 7573803 | 7574050 | 0.593 | TP53 |
| chr17 | 7573811 | 7574017 | 0.580 | TP53 |
| chr17 | 7576734 | 7576934 | 0.468 | TP53 |
| chr17 | 7576933 | 7577155 | 0.552 | TP53 |
| chr17 | 7576951 | 7577151 | 0.557 | TP53 |
| chr17 | 7576970 | 7577197 | 0.548 | TP53 |
| chr17 | 7576998 | 7577191 | 0.552 | TP53 |
| chr17 | 7576998 | 7577241 | 0.529 | TP53 |
| chr17 | 7576998 | 7577242 | 0.531 | TP53 |
| chr17 | 7577014 | 7577263 | 0.532 | TP53 |
| chr17 | 7577304 | 7577572 | 0.565 | TP53 |
| chr17 | 7577329 | 7577570 | 0.574 | TP53 |
| chr17 | 7577329 | 7577576 | 0.569 | TP53 |
| chr17 | 7577346 | 7577570 | 0.596 | TP53 |
| chr17 | 7577346 | 7577576 | 0.589 | TP53 |
| chr17 | 7577371 | 7577620 | 0.564 | TP53 |
| chr17 | 7577398 | 7577598 | 0.572 | TP53 |
| chr17 | 7578196 | 7578426 | 0.563 | TP53 |
| chr17 | 7578263 | 7578502 | 0.629 | TP53 |
| chr17 | 7578283 | 7578494 | 0.637 | TP53 |
| chr17 | 7578298 | 7578566 | 0.617 | TP53 |
| chr17 | 7578299 | 7578502 | 0.642 | TP53 |
| chr17 | 7578363 | 7578562 | 0.615 | TP53 |
| chr17 | 7578363 | 7578598 | 0.597 | TP53 |
| chr17 | 7579266 | 7579515 | 0.624 | TP53 |
| chr17 | 7579311 | 7579460 | 0.620 | TP53 |
| chr17 | 7579326 | 7579526 | 0.627 | TP53 |
| chr17 | 7579332 | 7579550 | 0.616 | TP53 |
| chr17 | 7579817 | 7579987 | 0.556 | TP53 |
| chr17 | 7590681 | 7590919 | 0.573 | TP53 |
| chr17 | 33426833 | 33427064 | 0.474 | RAD51D |
| chr17 | 33427044 | 33427283 | 0.500 | RAD51D |
| chr17 | 33427269 | 33427513 | 0.437 | RAD51D |
| chr17 | 33427477 | 33427725 | 0.518 | RAD51D |
| chr17 | 33427706 | 33427935 | 0.504 | RAD51D |
| chr17 | 33427916 | 33428162 | 0.498 | RAD51D |
| chr17 | 33428100 | 33428345 | 0.577 | RAD51D |
| chr17 | 33430196 | 33430419 | 0.567 | RAD51D |
| chr17 | 33430401 | 33430620 | 0.573 | RAD51D |
| chr17 | 33433344 | 33433506 | 0.564 | RAD51D |
| chr17 | 33433897 | 33434133 | 0.489 | RAD51D |
| chr17 | 33434295 | 33434532 | 0.500 | RAD51D |
| chr17 | 33445499 | 33445672 | 0.586 | RAD51D |
| chr17 | 33446550 | 33446780 | 0.662 | RAD51D |
| chr17 | 37868175 | 37868423 | 0.606 | ERBB2 |
| chr17 | 37868177 | 37868424 | 0.605 | ERBB2 |
| chr17 | 37868184 | 37868432 | 0.614 | ERBB2 |
| chr17 | 37868192 | 37868432 | 0.622 | ERBB2 |
| chr17 | 41226314 | 41226567 | 0.433 | BRCA1 |
| chr17 | 41228463 | 41228682 | 0.368 | BRCA1 |
| chr17 | 41231290 | 41231560 | 0.432 | BRCA1 |
| chr17 | 41234185 | 41234455 | 0.435 | BRCA1 |
| chr17 | 41234392 | 41234631 | 0.438 | BRCA1 |
| chr17 | 41242906 | 41243146 | 0.444 | BRCA1 |
| chr17 | 41243450 | 41243690 | 0.411 | BRCA1 |
| chr17 | 41243675 | 41243921 | 0.397 | BRCA1 |
| chr17 | 41243914 | 41244153 | 0.433 | BRCA1 |
| chr17 | 41244048 | 41244300 | 0.368 | BRCA1 |
| chr17 | 41244075 | 41244265 | 0.366 | BRCA1 |
| chr17 | 41244473 | 41244712 | 0.371 | BRCA1 |
| chr17 | 41244534 | 41244778 | 0.376 | BRCA1 |
| chr17 | 41244766 | 41245013 | 0.387 | BRCA1 |
| chr17 | 41245011 | 41245240 | 0.409 | BRCA1 |
| chr17 | 41245209 | 41245462 | 0.374 | BRCA1 |
| chr17 | 41245347 | 41245586 | 0.404 | BRCA1 |
| chr17 | 41245598 | 41245827 | 0.370 | BRCA1 |
| chr17 | 41245825 | 41246064 | 0.375 | BRCA1 |
| chr17 | 41246061 | 41246310 | 0.376 | BRCA1 |
| chr17 | 41246304 | 41246553 | 0.416 | BRCA1 |
| chr17 | 41246534 | 41246786 | 0.423 | BRCA1 |
| chr17 | 41246546 | 41246786 | 0.427 | BRCA1 |
| chr17 | 41246595 | 41246842 | 0.419 | BRCA1 |
| chr17 | 41246643 | 41246890 | 0.403 | BRCA1 |
| chr17 | 41246709 | 41246956 | 0.379 | BRCA1 |
| chr17 | 41246794 | 41247027 | 0.338 | BRCA1 |
| chr17 | 41247801 | 41248006 | 0.403 | BRCA1 |
| chr17 | 41249211 | 41249311 | 0.356 | BRCA1 |
| chr17 | 41251673 | 41251893 | 0.380 | BRCA1 |
| chr17 | 41251732 | 41251945 | 0.397 | BRCA1 |
| chr17 | 37880017 | 37880264 | 0.528 | ERBB2 |
| chr17 | 37880031 | 37880255 | 0.524 | ERBB2 |
| chr17 | 37880061 | 37880274 | 0.505 | ERBB2 |
| chr17 | 37880069 | 37880274 | 0.500 | ERBB2 |
| chr17 | 37880955 | 37881196 | 0.595 | ERBB2 |
| chr17 | 37880969 | 37881216 | 0.589 | ERBB2 |
| chr17 | 37880974 | 37881216 | 0.593 | ERBB2 |
| chr17 | 37880983 | 37881227 | 0.592 | ERBB2 |
| chr17 | 37881166 | 37881380 | 0.609 | ERBB2 |
| chr17 | 37881201 | 37881450 | 0.584 | ERBB2 |
| chr17 | 37881273 | 37881520 | 0.573 | ERBB2 |
| chr17 | 37881304 | 37881521 | 0.573 | ERBB2 |
| chr17 | 37881453 | 37881652 | 0.595 | ERBB2 |
| chr17 | 37881465 | 37881668 | 0.598 | ERBB2 |
| chr17 | 37881510 | 37881737 | 0.601 | ERBB2 |
| chr17 | 37881598 | 37881798 | 0.632 | ERBB2 |
| chr17 | 41196311 | 41196511 | 0.393 | BRCA1 |
| chr17 | 41197220 | 41197464 | 0.429 | BRCA1 |
| chr17 | 41197314 | 41197566 | 0.419 | BRCA1 |
| chr17 | 41197571 | 41197819 | 0.550 | BRCA1 |
| chr17 | 41199637 | 41199729 | 0.559 | BRCA1 |
| chr17 | 41201055 | 41201304 | 0.476 | BRCA1 |
| chr17 | 41202997 | 41203243 | 0.449 | BRCA1 |
| chr17 | 41209041 | 41209195 | 0.445 | BRCA1 |
| chr17 | 41215338 | 41215577 | 0.433 | BRCA1 |
| chr17 | 41215806 | 41216045 | 0.375 | BRCA1 |
| chr17 | 41219623 | 41219726 | 0.337 | BRCA1 |
| chr17 | 41222828 | 41223088 | 0.402 | BRCA1 |
| chr17 | 41222835 | 41223088 | 0.406 | BRCA1 |
| chr17 | 41222885 | 41223141 | 0.444 | BRCA1 |
| chr17 | 41223090 | 41223269 | 0.494 | BRCA1 |
| chr17 | 41256181 | 41256358 | 0.315 | BRCA1 |
| chr17 | 41256830 | 41257034 | 0.376 | BRCA1 |
| chr17 | 41256884 | 41257140 | 0.339 | BRCA1 |
| chr17 | 41258410 | 41258681 | 0.324 | BRCA1 |
| chr17 | 41267598 | 41267807 | 0.371 | BRCA1 |
| chr17 | 41267602 | 41267807 | 0.369 | BRCA1 |
| chr17 | 41267603 | 41267794 | 0.354 | BRCA1 |
| chr17 | 41267603 | 41267834 | 0.371 | BRCA1 |
| chr17 | 41267630 | 41267779 | 0.353 | BRCA1 |
| chr17 | 41267645 | 41267836 | 0.391 | BRCA1 |
| chr17 | 41275961 | 41276197 | 0.312 | BRCA1 |
| chr17 | 46805588 | 46805837 | 0.672 | HOXB13 |
| chr17 | 47696239 | 47696485 | 0.453 | SPOP |
| chr17 | 47696300 | 47696545 | 0.455 | SPOP |
| chr17 | 47696324 | 47696565 | 0.442 | SPOP |
| chr17 | 47696359 | 47696607 | 0.422 | SPOP |
| chr17 | 47696424 | 47696669 | 0.415 | SPOP |
| chr17 | 47696450 | 47696669 | 0.405 | SPOP |
| chr17 | 47696450 | 47696689 | 0.413 | SPOP |
| chr17 | 47696486 | 47696715 | 0.387 | SPOP |
| chr17 | 56769976 | 56770175 | 0.590 | RAD51C |
| chr17 | 56772289 | 56772542 | 0.421 | RAD51C |
| chr17 | 56773994 | 56774243 | 0.404 | RAD51C |
| chr17 | 56780486 | 56780723 | 0.345 | RAD51C |
| chr17 | 56787247 | 56787446 | 0.365 | RAD51C |
| chr17 | 56787285 | 56787460 | 0.341 | RAD51C |
| chr17 | 56798102 | 56798172 | 0.352 | RAD51C |
| chr17 | 56801331 | 56801553 | 0.390 | RAD51C |
| chr17 | 56809830 | 56810049 | 0.382 | RAD51C |
| chr17 | 56811478 | 56811716 | 0.385 | RAD51C |
| chr17 | 59756779 | 59756979 | 0.313 | BRIP1 |
| chr17 | 59759726 | 59759955 | 0.252 | BRIP1 |
| chr17 | 59760615 | 59760854 | 0.308 | BRIP1 |
| chr17 | 59760809 | 59761044 | 0.335 | BRIP1 |
| chr17 | 59761001 | 59761201 | 0.353 | BRIP1 |
| chr17 | 59761265 | 59761494 | 0.413 | BRIP1 |
| chr17 | 59761395 | 59761636 | 0.343 | BRIP1 |
| chr17 | 59763229 | 59763449 | 0.357 | BRIP1 |
| chr17 | 59763460 | 59763660 | 0.338 | BRIP1 |
| chr17 | 59770816 | 59771046 | 0.307 | BRIP1 |
| chr17 | 59793106 | 59793349 | 0.316 | BRIP1 |
| chr17 | 59793137 | 59793367 | 0.338 | BRIP1 |
| chr17 | 59820379 | 59820551 | 0.393 | BRIP1 |
| chr17 | 59821796 | 59821945 | 0.373 | BRIP1 |
| chr17 | 59853687 | 59853887 | 0.353 | BRIP1 |
| chr17 | 59857537 | 59857737 | 0.333 | BRIP1 |
| chr17 | 59858201 | 59858428 | 0.355 | BRIP1 |
| chr17 | 59861553 | 59861773 | 0.326 | BRIP1 |
| chr17 | 59870956 | 59871190 | 0.345 | BRIP1 |
| chr17 | 59876405 | 59876605 | 0.388 | BRIP1 |
| chr17 | 59876622 | 59876822 | 0.284 | BRIP1 |
| chr17 | 59878569 | 59878769 | 0.413 | BRIP1 |
| chr17 | 59878611 | 59878844 | 0.397 | BRIP1 |
| chr17 | 59885818 | 59886038 | 0.439 | BRIP1 |
| chr17 | 59886058 | 59886268 | 0.346 | BRIP1 |
| chr17 | 59924485 | 59924714 | 0.348 | BRIP1 |
| chr17 | 59926478 | 59926707 | 0.348 | BRIP1 |
| chr17 | 59934381 | 59934581 | 0.383 | BRIP1 |
| chr17 | 59937155 | 59937392 | 0.374 | BRIP1 |
| chr17 | 59938714 | 59938914 | 0.333 | BRIP1 |
| chr17 | 59940627 | 59940827 | 0.577 | BRIP1 |
| chr17 | 59940844 | 59941054 | 0.592 | BRIP1 |
| chr18 | 48556368 | 48556604 | 0.692 | SMAD4 |
| chr18 | 48556368 | 48556608 | 0.689 | SMAD4 |
| chr18 | 48556413 | 48556612 | 0.680 | SMAD4 |
| chr18 | 48556414 | 48556616 | 0.675 | SMAD4 |
| chr18 | 48573249 | 48573492 | 0.328 | SMAD4 |
| chr18 | 48573500 | 48573738 | 0.364 | SMAD4 |
| chr18 | 48575147 | 48575346 | 0.340 | SMAD4 |
| chr18 | 48575524 | 48575724 | 0.294 | SMAD4 |
| chr18 | 48581203 | 48581433 | 0.455 | SMAD4 |
| chr18 | 48581249 | 48581498 | 0.428 | SMAD4 |
| chr18 | 48584399 | 48584599 | 0.443 | SMAD4 |
| chr18 | 48584673 | 48584893 | 0.439 | SMAD4 |
| chr18 | 48586106 | 48586306 | 0.308 | SMAD4 |
| chr18 | 48591791 | 48592035 | 0.408 | SMAD4 |
| chr18 | 48593378 | 48593608 | 0.407 | SMAD4 |
| chr18 | 48602902 | 48603166 | 0.430 | SMAD4 |
| chr18 | 48602922 | 48603132 | 0.445 | SMAD4 |
| chr18 | 48603000 | 48603249 | 0.424 | SMAD4 |
| chr18 | 48603132 | 48603332 | 0.289 | SMAD4 |
| chr18 | 48604566 | 48604815 | 0.488 | SMAD4 |
| chr18 | 48604617 | 48604861 | 0.490 | SMAD4 |
| chr18 | 48604640 | 48604881 | 0.492 | SMAD4 |
| chr18 | 48604711 | 48604957 | 0.421 | SMAD4 |
| chr18 | 48605303 | 48605503 | 0.294 | SMAD4 |
| chr18 | 48605551 | 48605798 | 0.351 | SMAD4 |
| chr18 | 48605981 | 48606181 | 0.318 | SMAD4 |
| chr18 | 48606203 | 48606477 | 0.349 | SMAD4 |
| chr18 | 48606469 | 48606712 | 0.357 | SMAD4 |
| chr18 | 48607311 | 48607586 | 0.326 | SMAD4 |
| chr18 | 48607638 | 48607926 | 0.412 | SMAD4 |
| chr18 | 48608009 | 48608208 | 0.375 | SMAD4 |
| chr18 | 48608225 | 48608485 | 0.368 | SMAD4 |
| chr18 | 48608754 | 48608904 | 0.450 | SMAD4 |
| chr18 | 48609535 | 48609685 | 0.470 | SMAD4 |
| chr18 | 48609670 | 48609869 | 0.355 | SMAD4 |
| chr18 | 48610653 | 48610803 | 0.358 | SMAD4 |
| chr18 | 48610833 | 48611078 | 0.488 | SMAD4 |
| chr18 | 48611097 | 48611339 | 0.490 | SMAD4 |
| chr18 | 48611405 | 48611605 | 0.353 | SMAD4 |
| chr19 | 1206543 | 1206797 | 0.643 | STK11 |
| chr19 | 1206613 | 1206843 | 0.558 | STK11 |
| chr19 | 1206770 | 1207017 | 0.560 | STK11 |
| chr19 | 1206962 | 1207206 | 0.596 | STK11 |
| chr19 | 1218302 | 1218502 | 0.532 | STK11 |
| chr19 | 1219161 | 1219399 | 0.653 | STK11 |
| chr19 | 1220251 | 1220492 | 0.632 | STK11 |
| chr19 | 1220276 | 1220492 | 0.636 | STK11 |
| chr19 | 1220475 | 1220701 | 0.705 | STK11 |
| chr19 | 1220502 | 1220701 | 0.715 | STK11 |
| chr19 | 1220681 | 1220938 | 0.647 | STK11 |
| chr19 | 1220681 | 1220941 | 0.648 | STK11 |
| chr19 | 1221151 | 1221351 | 0.582 | STK11 |
| chr19 | 1221821 | 1222071 | 0.685 | STK11 |
| chr19 | 1222984 | 1223224 | 0.643 | STK11 |
| chr19 | 1226453 | 1226708 | 0.715 | STK11 |
| chr19 | 1226464 | 1226754 | 0.704 | STK11 |
| chr19 | 1226465 | 1226707 | 0.716 | STK11 |
| chr19 | 1228261 | 1228461 | 0.547 | STK11 |
| chr19 | 3114798 | 3115046 | 0.683 | GNA11 |
| chr19 | 3114798 | 3115049 | 0.683 | GNA11 |
| chr19 | 3114839 | 3115040 | 0.688 | GNA11 |
| chr19 | 3114841 | 3115040 | 0.685 | GNA11 |
| chr19 | 3118772 | 3118996 | 0.622 | GNA11 |
| chr19 | 3118795 | 3119044 | 0.620 | GNA11 |
| chr19 | 3118818 | 3119047 | 0.613 | GNA11 |
| chr19 | 3118863 | 3119109 | 0.636 | GNA11 |
| chr19 | 10600276 | 10600491 | 0.588 | KEAP1 |
| chr19 | 10600284 | 10600528 | 0.592 | KEAP1 |
| chr19 | 10600284 | 10600532 | 0.594 | KEAP1 |
| chr19 | 10602242 | 10602441 | 0.645 | KEAP1 |
| chr19 | 10602423 | 10602671 | 0.691 | KEAP1 |
| chr19 | 10602539 | 10602747 | 0.632 | KEAP1 |
| chr19 | 10602539 | 10602753 | 0.628 | KEAP1 |
| chr19 | 10602653 | 10602854 | 0.609 | KEAP1 |
| chr19 | 10610056 | 10610289 | 0.585 | KEAP1 |
| chr19 | 10610069 | 10610306 | 0.576 | KEAP1 |
| chr19 | 10610081 | 10610306 | 0.580 | KEAP1 |
| chr19 | 10610083 | 10610306 | 0.580 | KEAP1 |
| chr19 | 11094768 | 11095038 | 0.697 | SMARCA4 |
| chr19 | 11095950 | 11096150 | 0.592 | SMARCA4 |
| chr19 | 11096820 | 11097078 | 0.625 | SMARCA4 |
| chr19 | 11097475 | 11097685 | 0.645 | SMARCA4 |
| chr19 | 11098265 | 11098553 | 0.706 | SMARCA4 |
| chr19 | 11098298 | 11098538 | 0.726 | SMARCA4 |
| chr19 | 11098376 | 11098606 | 0.732 | SMARCA4 |
| chr19 | 11099901 | 11100111 | 0.607 | SMARCA4 |
| chr19 | 11101757 | 11101957 | 0.612 | SMARCA4 |
| chr19 | 11105466 | 11105666 | 0.522 | SMARCA4 |
| chr19 | 11106694 | 11106939 | 0.557 | SMARCA4 |
| chr19 | 11107143 | 11107352 | 0.476 | SMARCA4 |
| chr19 | 11113645 | 11113845 | 0.592 | SMARCA4 |
| chr19 | 11113855 | 11114055 | 0.493 | SMARCA4 |
| chr19 | 11118483 | 11118683 | 0.587 | SMARCA4 |
| chr19 | 11120977 | 11121177 | 0.557 | SMARCA4 |
| chr19 | 11123551 | 11123751 | 0.592 | SMARCA4 |
| chr19 | 11129571 | 11129781 | 0.555 | SMARCA4 |
| chr19 | 11130134 | 11130372 | 0.619 | SMARCA4 |
| chr19 | 11130167 | 11130367 | 0.612 | SMARCA4 |
| chr19 | 11130245 | 11130468 | 0.585 | SMARCA4 |
| chr19 | 11130248 | 11130473 | 0.584 | SMARCA4 |
| chr19 | 11132367 | 11132587 | 0.611 | SMARCA4 |
| chr19 | 11132607 | 11132807 | 0.622 | SMARCA4 |
| chr19 | 11134126 | 11134326 | 0.557 | SMARCA4 |
| chr19 | 11134905 | 11135105 | 0.617 | SMARCA4 |
| chr19 | 11135986 | 11136186 | 0.602 | SMARCA4 |
| chr19 | 11136874 | 11137104 | 0.610 | SMARCA4 |
| chr19 | 11138402 | 11138674 | 0.502 | SMARCA4 |
| chr19 | 11138598 | 11138818 | 0.597 | SMARCA4 |
| chr19 | 11141332 | 11141532 | 0.637 | SMARCA4 |
| chr19 | 11141549 | 11141749 | 0.627 | SMARCA4 |
| chr19 | 11143924 | 11144134 | 0.621 | SMARCA4 |
| chr19 | 11144041 | 11144262 | 0.617 | SMARCA4 |
| chr19 | 11144868 | 11145108 | 0.618 | SMARCA4 |
| chr19 | 11145552 | 11145823 | 0.654 | SMARCA4 |
| chr19 | 11151919 | 11152129 | 0.616 | SMARCA4 |
| chr19 | 11151919 | 11152189 | 0.627 | SMARCA4 |
| chr19 | 11152171 | 11152371 | 0.572 | SMARCA4 |
| chr19 | 11168890 | 11169140 | 0.625 | SMARCA4 |
| chr19 | 11169359 | 11169619 | 0.644 | SMARCA4 |
| chr19 | 11170610 | 11170850 | 0.668 | SMARCA4 |
| chr19 | 11172452 | 11172706 | 0.498 | SMARCA4 |
| chr19 | 11172557 | 11172757 | 0.423 | SMARCA4 |
| chr19 | 11172753 | 11172953 | 0.493 | SMARCA4 |
| chr19 | 45854611 | 45854870 | 0.581 | ERCC2 |
| chr19 | 45854917 | 45855123 | 0.589 | ERCC2 |
| chr19 | 45855406 | 45855649 | 0.656 | ERCC2 |
| chr19 | 45855711 | 45855931 | 0.611 | ERCC2 |
| chr19 | 45855908 | 45856123 | 0.611 | ERCC2 |
| chr19 | 45855910 | 45856123 | 0.612 | ERCC2 |
| chr19 | 45855914 | 45856123 | 0.610 | ERCC2 |
| chr19 | 45855948 | 45856167 | 0.609 | ERCC2 |
| chr19 | 45855959 | 45856169 | 0.607 | ERCC2 |
| chr19 | 45856222 | 45856442 | 0.652 | ERCC2 |
| chr19 | 45857893 | 45858113 | 0.597 | ERCC2 |
| chr19 | 45860528 | 45860730 | 0.635 | ERCC2 |
| chr19 | 45860797 | 45861007 | 0.635 | ERCC2 |
| chr19 | 45864772 | 45864905 | 0.575 | ERCC2 |
| chr19 | 45866937 | 45867137 | 0.706 | ERCC2 |
| chr19 | 45867119 | 45867343 | 0.724 | ERCC2 |
| chr19 | 45867124 | 45867373 | 0.716 | ERCC2 |
| chr19 | 45867329 | 45867584 | 0.684 | ERCC2 |
| chr19 | 45867491 | 45867711 | 0.656 | ERCC2 |
| chr19 | 45867506 | 45867746 | 0.660 | ERCC2 |
| chr19 | 45867566 | 45867806 | 0.618 | ERCC2 |
| chr19 | 45868096 | 45868344 | 0.635 | ERCC2 |
| chr19 | 45868149 | 45868349 | 0.637 | ERCC2 |
| chr19 | 45868154 | 45868396 | 0.626 | ERCC2 |
| chr19 | 45868287 | 45868486 | 0.600 | ERCC2 |
| chr19 | 45871786 | 45871991 | 0.524 | ERCC2 |
| chr19 | 45872064 | 45872264 | 0.582 | ERCC2 |
| chr19 | 45872211 | 45872411 | 0.562 | ERCC2 |
| chr19 | 45873397 | 45873585 | 0.661 | ERCC2 |
| chr19 | 45873421 | 45873651 | 0.675 | ERCC2 |
| chr19 | 45873436 | 45873665 | 0.687 | ERCC2 |
| chr19 | 45873632 | 45873861 | 0.700 | ERCC2 |
| chr19 | 45873636 | 45873901 | 0.684 | ERCC2 |
| chr19 | 45873726 | 45873936 | 0.645 | ERCC2 |
| chr19 | 45916987 | 45917191 | 0.571 | ERCC1 |
| chr19 | 45918047 | 45918240 | 0.619 | ERCC1 |
| chr19 | 45918047 | 45918243 | 0.619 | ERCC1 |
| chr19 | 45918048 | 45918243 | 0.622 | ERCC1 |
| chr19 | 45918053 | 45918243 | 0.628 | ERCC1 |
| chr19 | 45918060 | 45918235 | 0.625 | ERCC1 |
| chr19 | 45922222 | 45922436 | 0.567 | ERCC1 |
| chr19 | 45923506 | 45923678 | 0.607 | ERCC1 |
| chr19 | 45924445 | 45924632 | 0.638 | ERCC1 |
| chr19 | 45926611 | 45926815 | 0.615 | ERCC1 |
| chr19 | 50902097 | 50902336 | 0.650 | POLD1 |
| chr19 | 50902458 | 50902658 | 0.582 | POLD1 |
| chr19 | 50904960 | 50905171 | 0.656 | POLD1 |
| chr19 | 50905153 | 50905392 | 0.671 | POLD1 |
| chr19 | 50905436 | 50905665 | 0.683 | POLD1 |
| chr19 | 50905615 | 50905913 | 0.659 | POLD1 |
| chr19 | 50905899 | 50906137 | 0.665 | POLD1 |
| chr19 | 50906253 | 50906464 | 0.679 | POLD1 |
| chr19 | 50906723 | 50906971 | 0.622 | POLD1 |
| chr19 | 50906755 | 50906971 | 0.613 | POLD1 |
| chr19 | 50909485 | 50909773 | 0.640 | POLD1 |
| chr19 | 50910320 | 50910533 | 0.631 | POLD1 |
| chr19 | 50910334 | 50910578 | 0.629 | POLD1 |
| chr19 | 50910376 | 50910627 | 0.631 | POLD1 |
| chr19 | 50912042 | 50912281 | 0.617 | POLD1 |
| chr19 | 50912288 | 50912520 | 0.635 | POLD1 |
| chr19 | 50912795 | 50913010 | 0.667 | POLD1 |
| chr19 | 50916709 | 50916950 | 0.640 | POLD1 |
| chr19 | 50916970 | 50917221 | 0.623 | POLD1 |
| chr19 | 50917955 | 50918169 | 0.628 | POLD1 |
| chr19 | 50917960 | 50918169 | 0.624 | POLD1 |
| chr19 | 50918653 | 50918824 | 0.640 | POLD1 |
| chr19 | 50919033 | 50919269 | 0.692 | POLD1 |
| chr19 | 50919496 | 50919745 | 0.684 | POLD1 |
| chr19 | 50919683 | 50919957 | 0.691 | POLD1 |
| chr19 | 50919685 | 50919957 | 0.689 | POLD1 |
| chr19 | 50919770 | 50920048 | 0.699 | POLD1 |
| chr19 | 50919821 | 50920050 | 0.691 | POLD1 |
| chr19 | 50921104 | 50921313 | 0.576 | POLD1 |
| chr2 | 29443543 | 29443787 | 0.539 | ALK |
| chr2 | 29443549 | 29443783 | 0.545 | ALK |
| chr2 | 29443555 | 29443789 | 0.540 | ALK |
| chr2 | 29443585 | 29443816 | 0.500 | ALK |
| chr2 | 29445100 | 29445340 | 0.552 | ALK |
| chr2 | 29445107 | 29445340 | 0.556 | ALK |
| chr2 | 29445121 | 29445350 | 0.570 | ALK |
| chr2 | 29445121 | 29445367 | 0.571 | ALK |
| chr2 | 47600500 | 47600700 | 0.323 | EPCAM |
| chr2 | 47600912 | 47601112 | 0.473 | EPCAM |
| chr2 | 47601122 | 47601322 | 0.383 | EPCAM |
| chr2 | 47602199 | 47602445 | 0.328 | EPCAM |
| chr2 | 47604131 | 47604282 | 0.342 | EPCAM |
| chr2 | 47605987 | 47606187 | 0.249 | EPCAM |
| chr2 | 47606812 | 47606991 | 0.361 | EPCAM |
| chr2 | 47612268 | 47612488 | 0.398 | EPCAM |
| chr2 | 47613710 | 47613910 | 0.343 | EPCAM |
| chr2 | 47630105 | 47630305 | 0.652 | MSH2 |
| chr2 | 47630152 | 47630400 | 0.643 | MSH2 |
| chr2 | 47630268 | 47630467 | 0.615 | MSH2 |
| chr2 | 47630315 | 47630515 | 0.667 | MSH2 |
| chr2 | 47630316 | 47630530 | 0.647 | MSH2 |
| chr2 | 47630384 | 47630615 | 0.690 | MSH2 |
| chr2 | 47630425 | 47630625 | 0.706 | MSH2 |
| chr2 | 47635539 | 47635709 | 0.339 | MSH2 |
| chr2 | 47637342 | 47637582 | 0.456 | MSH2 |
| chr2 | 47637389 | 47637594 | 0.442 | MSH2 |
| chr2 | 47639447 | 47639662 | 0.310 | MSH2 |
| chr2 | 47641283 | 47641487 | 0.327 | MSH2 |
| chr2 | 47643346 | 47643546 | 0.398 | MSH2 |
| chr2 | 47656945 | 47657096 | 0.388 | MSH2 |
| chr2 | 47672586 | 47672786 | 0.299 | MSH2 |
| chr2 | 47690076 | 47690276 | 0.303 | MSH2 |
| chr2 | 47693747 | 47693947 | 0.348 | MSH2 |
| chr2 | 47698159 | 47698397 | 0.326 | MSH2 |
| chr2 | 47702121 | 47702341 | 0.394 | MSH2 |
| chr2 | 47702174 | 47702413 | 0.388 | MSH2 |
| chr2 | 47703453 | 47703653 | 0.428 | MSH2 |
| chr2 | 47703663 | 47703863 | 0.363 | MSH2 |
| chr2 | 47705399 | 47705630 | 0.397 | MSH2 |
| chr2 | 47707788 | 47708018 | 0.424 | MSH2 |
| chr2 | 47709903 | 47710113 | 0.327 | MSH2 |
| chr2 | 47710107 | 47710336 | 0.287 | MSH2 |
| chr2 | 48010393 | 48010592 | 0.725 | MSH6 |
| chr2 | 48010420 | 48010619 | 0.740 | MSH6 |
| chr2 | 48010573 | 48010824 | 0.690 | MSH6 |
| chr2 | 48010575 | 48010781 | 0.725 | MSH6 |
| chr2 | 48017953 | 48018200 | 0.419 | MSH6 |
| chr2 | 48018205 | 48018415 | 0.431 | MSH6 |
| chr2 | 48023040 | 48023272 | 0.455 | MSH6 |
| chr2 | 48023105 | 48023304 | 0.430 | MSH6 |
| chr2 | 48025749 | 48025949 | 0.428 | MSH6 |
| chr2 | 48025933 | 48026201 | 0.476 | MSH6 |
| chr2 | 48026177 | 48026376 | 0.460 | MSH6 |
| chr2 | 48026340 | 48026558 | 0.457 | MSH6 |
| chr2 | 48026511 | 48026711 | 0.458 | MSH6 |
| chr2 | 48026765 | 48026965 | 0.383 | MSH6 |
| chr2 | 48027019 | 48027219 | 0.438 | MSH6 |
| chr2 | 48027273 | 48027473 | 0.438 | MSH6 |
| chr2 | 48027527 | 48027727 | 0.398 | MSH6 |
| chr2 | 48027746 | 48028017 | 0.426 | MSH6 |
| chr2 | 48028124 | 48028364 | 0.415 | MSH6 |
| chr2 | 48030536 | 48030736 | 0.433 | MSH6 |
| chr2 | 48030729 | 48031008 | 0.371 | MSH6 |
| chr2 | 48031952 | 48032152 | 0.443 | MSH6 |
| chr2 | 48032026 | 48032237 | 0.420 | MSH6 |
| chr2 | 48032772 | 48032971 | 0.315 | MSH6 |
| chr2 | 48033358 | 48033554 | 0.365 | MSH6 |
| chr2 | 48033416 | 48033655 | 0.367 | MSH6 |
| chr2 | 48033581 | 48033782 | 0.401 | MSH6 |
| chr2 | 48034024 | 48034264 | 0.307 | MSH6 |
| chr2 | 58386432 | 58386632 | 0.333 | FANCL |
| chr2 | 58386777 | 58386929 | 0.307 | FANCL |
| chr2 | 58387189 | 58387389 | 0.338 | FANCL |
| chr2 | 58388496 | 58388732 | 0.346 | FANCL |
| chr2 | 58389905 | 58390169 | 0.328 | FANCL |
| chr2 | 58390005 | 58390214 | 0.352 | FANCL |
| chr2 | 58390571 | 58390773 | 0.379 | FANCL |
| chr2 | 58392779 | 58392979 | 0.398 | FANCL |
| chr2 | 58431158 | 58431358 | 0.363 | FANCL |
| chr2 | 58448972 | 58449172 | 0.323 | FANCL |
| chr2 | 58453736 | 58453936 | 0.308 | FANCL |
| chr2 | 58456824 | 58457044 | 0.285 | FANCL |
| chr2 | 58459138 | 58459342 | 0.351 | FANCL |
| chr2 | 58468279 | 58468479 | 0.592 | FANCL |
| chr2 | 128014784 | 128015047 | 0.394 | ERCC3 |
| chr2 | 128015087 | 128015289 | 0.522 | ERCC3 |
| chr2 | 128016793 | 128017023 | 0.541 | ERCC3 |
| chr2 | 128018729 | 128018934 | 0.476 | ERCC3 |
| chr2 | 128028846 | 128029046 | 0.493 | ERCC3 |
| chr2 | 128030334 | 128030534 | 0.488 | ERCC3 |
| chr2 | 128036695 | 128036895 | 0.413 | ERCC3 |
| chr2 | 128037952 | 128038168 | 0.535 | ERCC3 |
| chr2 | 128044271 | 128044501 | 0.580 | ERCC3 |
| chr2 | 128044494 | 128044693 | 0.500 | ERCC3 |
| chr2 | 128046183 | 128046383 | 0.502 | ERCC3 |
| chr2 | 128046840 | 128047040 | 0.468 | ERCC3 |
| chr2 | 128047177 | 128047377 | 0.522 | ERCC3 |
| chr2 | 128047669 | 128047869 | 0.448 | ERCC3 |
| chr2 | 128050206 | 128050419 | 0.514 | ERCC3 |
| chr2 | 128051102 | 128051343 | 0.566 | ERCC3 |
| chr2 | 128051633 | 128051852 | 0.655 | ERCC3 |
| chr2 | 128051746 | 128051946 | 0.577 | ERCC3 |
| chr2 | 209112977 | 209113230 | 0.394 | IDH1 |
| chr2 | 209113091 | 209113340 | 0.444 | IDH1 |
| chr2 | 212288833 | 212289075 | 0.366 | ERBB4 |
| chr2 | 212288836 | 212289075 | 0.367 | ERBB4 |
| chr2 | 212288849 | 212289089 | 0.378 | ERBB4 |
| chr2 | 212288867 | 212289111 | 0.376 | ERBB4 |
| chr2 | 212483732 | 212483976 | 0.327 | ERBB4 |
| chr2 | 212483745 | 212483975 | 0.338 | ERBB4 |
| chr2 | 212483745 | 212483989 | 0.343 | ERBB4 |
| chr2 | 212483745 | 212483992 | 0.347 | ERBB4 |
| chr2 | 212529983 | 212530215 | 0.468 | ERBB4 |
| chr2 | 212529983 | 212530219 | 0.468 | ERBB4 |
| chr2 | 212530006 | 212530255 | 0.444 | ERBB4 |
| chr2 | 212530049 | 212530293 | 0.420 | ERBB4 |
| chr2 | 212587102 | 212587341 | 0.379 | ERBB4 |
| chr2 | 212587102 | 212587342 | 0.378 | ERBB4 |
| chr2 | 212587104 | 212587343 | 0.379 | ERBB4 |
| chr2 | 215591713 | 215591913 | 0.348 | BARD1 |
| chr2 | 215592049 | 215592249 | 0.313 | BARD1 |
| chr2 | 215592385 | 215592585 | 0.318 | BARD1 |
| chr2 | 215592721 | 215592921 | 0.284 | BARD1 |
| chr2 | 215593393 | 215593593 | 0.458 | BARD1 |
| chr2 | 215593464 | 215593712 | 0.482 | BARD1 |
| chr2 | 215595070 | 215595319 | 0.348 | BARD1 |
| chr2 | 215609774 | 215610018 | 0.331 | BARD1 |
| chr2 | 215610357 | 215610557 | 0.368 | BARD1 |
| chr2 | 215617201 | 215617402 | 0.322 | BARD1 |
| chr2 | 215617244 | 215617478 | 0.315 | BARD1 |
| chr2 | 215632137 | 215632347 | 0.398 | BARD1 |
| chr2 | 215632347 | 215632547 | 0.299 | BARD1 |
| chr2 | 215633841 | 215634041 | 0.348 | BARD1 |
| chr2 | 215645283 | 215645483 | 0.418 | BARD1 |
| chr2 | 215645568 | 215645768 | 0.398 | BARD1 |
| chr2 | 215645789 | 215646022 | 0.385 | BARD1 |
| chr2 | 215645997 | 215646196 | 0.405 | BARD1 |
| chr2 | 215646018 | 215646167 | 0.413 | BARD1 |
| chr2 | 215656994 | 215657164 | 0.421 | BARD1 |
| chr2 | 215661815 | 215662059 | 0.355 | BARD1 |
| chr2 | 215674040 | 215674299 | 0.662 | BARD1 |
| chr2 | 215674057 | 215674299 | 0.671 | BARD1 |
| chr2 | 215674060 | 215674299 | 0.675 | BARD1 |
| chr2 | 215674115 | 215674321 | 0.681 | BARD1 |
| chr20 | 57484302 | 57484538 | 0.439 | GNAS |
| chr21 | 36252718 | 36252963 | 0.427 | RUNX1 |
| chr21 | 36252753 | 36253001 | 0.454 | RUNX1 |
| chr21 | 36252796 | 36253037 | 0.471 | RUNX1 |
| chr21 | 36252819 | 36253063 | 0.469 | RUNX1 |
| chr22 | 29092793 | 29093014 | 0.383 | CHEK2 |
| chr22 | 29095766 | 29095985 | 0.468 | CHEK2 |
| chr22 | 29099378 | 29099614 | 0.354 | CHEK2 |
| chr22 | 29105946 | 29106126 | 0.243 | CHEK2 |
| chr22 | 29105988 | 29106140 | 0.288 | CHEK2 |
| chr22 | 29107796 | 29107996 | 0.373 | CHEK2 |
| chr22 | 29115374 | 29115613 | 0.292 | CHEK2 |
| chr22 | 29120968 | 29121207 | 0.358 | CHEK2 |
| chr22 | 29121185 | 29121429 | 0.376 | CHEK2 |
| chr22 | 29130538 | 29130762 | 0.556 | CHEK2 |
| chr22 | 29130552 | 29130805 | 0.508 | CHEK2 |
| chr22 | 29137634 | 29137834 | 0.547 | CHEK2 |
| chr3 | 10070202 | 10070402 | 0.383 | FANCD2 |
| chr3 | 10074431 | 10074641 | 0.303 | FANCD2 |
| chr3 | 10076444 | 10076673 | 0.430 | FANCD2 |
| chr3 | 10076732 | 10076932 | 0.323 | FANCD2 |
| chr3 | 10077981 | 10078130 | 0.313 | FANCD2 |
| chr3 | 10080961 | 10081110 | 0.367 | FANCD2 |
| chr3 | 10081391 | 10081592 | 0.510 | FANCD2 |
| chr3 | 10083255 | 10083471 | 0.433 | FANCD2 |
| chr3 | 10116194 | 10116415 | 0.405 | FANCD2 |
| chr3 | 10119764 | 10119916 | 0.523 | FANCD2 |
| chr3 | 10122693 | 10122903 | 0.422 | FANCD2 |
| chr3 | 10122903 | 10123103 | 0.378 | FANCD2 |
| chr3 | 10123144 | 10123344 | 0.358 | FANCD2 |
| chr3 | 10127494 | 10127703 | 0.433 | FANCD2 |
| chr3 | 10128659 | 10128873 | 0.414 | FANCD2 |
| chr3 | 10130055 | 10130255 | 0.448 | FANCD2 |
| chr3 | 10130418 | 10130618 | 0.398 | FANCD2 |
| chr3 | 10131860 | 10132052 | 0.461 | FANCD2 |
| chr3 | 10133776 | 10133976 | 0.418 | FANCD2 |
| chr3 | 10134833 | 10135033 | 0.448 | FANCD2 |
| chr3 | 10135971 | 10136220 | 0.484 | FANCD2 |
| chr3 | 10136796 | 10137016 | 0.425 | FANCD2 |
| chr3 | 10137928 | 10138128 | 0.398 | FANCD2 |
| chr3 | 10140403 | 10140603 | 0.448 | FANCD2 |
| chr3 | 10140685 | 10140895 | 0.322 | FANCD2 |
| chr3 | 10183302 | 10183451 | 0.653 | VHL |
| chr3 | 10183681 | 10183874 | 0.706 | VHL |
| chr3 | 10188234 | 10188438 | 0.390 | VHL |
| chr3 | 10191445 | 10191700 | 0.484 | VHL |
| chr3 | 10191721 | 10191932 | 0.392 | VHL |
| chr3 | 10192245 | 10192450 | 0.350 | VHL |
| chr3 | 12645599 | 12645838 | 0.504 | RAF1 |
| chr3 | 12645599 | 12645843 | 0.502 | RAF1 |
| chr3 | 12645599 | 12645844 | 0.500 | RAF1 |
| chr3 | 12645603 | 12645844 | 0.504 | RAF1 |
| chr3 | 14186692 | 14186898 | 0.319 | XPC |
| chr3 | 14186863 | 14187104 | 0.521 | XPC |
| chr3 | 14187085 | 14187334 | 0.532 | XPC |
| chr3 | 14187312 | 14187552 | 0.589 | XPC |
| chr3 | 14187523 | 14187722 | 0.570 | XPC |
| chr3 | 14188679 | 14188879 | 0.552 | XPC |
| chr3 | 14189299 | 14189509 | 0.583 | XPC |
| chr3 | 14189991 | 14190191 | 0.597 | XPC |
| chr3 | 14190286 | 14190496 | 0.578 | XPC |
| chr3 | 14193741 | 14194025 | 0.596 | XPC |
| chr3 | 14197850 | 14198050 | 0.483 | XPC |
| chr3 | 14199573 | 14199852 | 0.543 | XPC |
| chr3 | 14199862 | 14200062 | 0.582 | XPC |
| chr3 | 14200146 | 14200394 | 0.522 | XPC |
| chr3 | 14206322 | 14206487 | 0.434 | XPC |
| chr3 | 14206933 | 14207089 | 0.522 | XPC |
| chr3 | 14208699 | 14208911 | 0.474 | XPC |
| chr3 | 14209663 | 14209863 | 0.517 | XPC |
| chr3 | 14211839 | 14212049 | 0.384 | XPC |
| chr3 | 14214355 | 14214600 | 0.476 | XPC |
| chr3 | 14220004 | 14220204 | 0.682 | XPC |
| chr3 | 37034589 | 37034809 | 0.570 | EPM2AIP1 |
| chr3 | 37034790 | 37035063 | 0.544 | EPM2AIP1 |
| chr3 | 37035069 | 37035306 | 0.622 | MLH1 |
| chr3 | 37038000 | 37038200 | 0.368 | MLH1 |
| chr3 | 37042434 | 37042645 | 0.354 | MLH1 |
| chr3 | 37045773 | 37045973 | 0.418 | MLH1 |
| chr3 | 37048411 | 37048645 | 0.353 | MLH1 |
| chr3 | 37050230 | 37050436 | 0.377 | MLH1 |
| chr3 | 37053207 | 37053427 | 0.335 | MLH1 |
| chr3 | 37053528 | 37053730 | 0.340 | MLH1 |
| chr3 | 37055893 | 37056093 | 0.373 | MLH1 |
| chr3 | 37058865 | 37059114 | 0.424 | MLH1 |
| chr3 | 37061804 | 37062039 | 0.542 | MLH1 |
| chr3 | 37067171 | 37067392 | 0.500 | MLH1 |
| chr3 | 37067236 | 37067492 | 0.514 | MLH1 |
| chr3 | 37070194 | 37070394 | 0.408 | MLH1 |
| chr3 | 37070355 | 37070605 | 0.462 | MLH1 |
| chr3 | 37081625 | 37081777 | 0.438 | MLH1 |
| chr3 | 37083681 | 37083889 | 0.354 | MLH1 |
| chr3 | 37088877 | 37089113 | 0.468 | MLH1 |
| chr3 | 37088933 | 37089152 | 0.473 | MLH1 |
| chr3 | 37089899 | 37090099 | 0.468 | MLH1 |
| chr3 | 37090327 | 37090527 | 0.428 | MLH1 |
| chr3 | 37091892 | 37092087 | 0.480 | MLH1 |
| chr3 | 37091894 | 37092142 | 0.466 | MLH1 |
| chr3 | 41265974 | 41266223 | 0.464 | CTNNB1 |
| chr3 | 41266013 | 41266252 | 0.488 | CTNNB1 |
| chr3 | 41266021 | 41266268 | 0.476 | CTNNB1 |
| chr3 | 41266036 | 41266265 | 0.474 | CTNNB1 |
| chr3 | 52435062 | 52435320 | 0.583 | BAP1 |
| chr3 | 52435350 | 52435631 | 0.543 | BAP1 |
| chr3 | 52435934 | 52436211 | 0.561 | BAP1 |
| chr3 | 52436217 | 52436465 | 0.647 | BAP1 |
| chr3 | 52436552 | 52436790 | 0.552 | BAP1 |
| chr3 | 52436807 | 52437063 | 0.572 | BAP1 |
| chr3 | 52437127 | 52437370 | 0.574 | BAP1 |
| chr3 | 52437447 | 52437693 | 0.599 | BAP1 |
| chr3 | 52437754 | 52437978 | 0.556 | BAP1 |
| chr3 | 52438443 | 52438682 | 0.575 | BAP1 |
| chr3 | 52439130 | 52439357 | 0.583 | BAP1 |
| chr3 | 52439752 | 52440014 | 0.544 | BAP1 |
| chr3 | 52440273 | 52440505 | 0.618 | BAP1 |
| chr3 | 52440695 | 52440946 | 0.552 | BAP1 |
| chr3 | 52441181 | 52441475 | 0.576 | BAP1 |
| chr3 | 52441983 | 52442230 | 0.532 | BAP1 |
| chr3 | 52442460 | 52442693 | 0.500 | BAP1 |
| chr3 | 52443505 | 52443740 | 0.602 | BAP1 |
| chr3 | 138374171 | 138374400 | 0.400 | PIK3CB |
| chr3 | 138374183 | 138374428 | 0.386 | PIK3CB |
| chr3 | 138374197 | 138374427 | 0.398 | PIK3CB |
| chr3 | 138374204 | 138374443 | 0.408 | PIK3CB |
| chr3 | 138409848 | 138410087 | 0.375 | PIK3CB |
| chr3 | 138409848 | 138410097 | 0.380 | PIK3CB |
| chr3 | 138409872 | 138410116 | 0.380 | PIK3CB |
| chr3 | 138409872 | 138410118 | 0.385 | PIK3CB |
| chr3 | 138417690 | 138417920 | 0.390 | PIK3CB |
| chr3 | 138417690 | 138417924 | 0.383 | PIK3CB |
| chr3 | 138417696 | 138417915 | 0.391 | PIK3CB |
| chr3 | 138417697 | 138417916 | 0.395 | PIK3CB |
| chr3 | 138665132 | 138665361 | 0.600 | FOXL2 |
| chr3 | 138665147 | 138665396 | 0.600 | FOXL2 |
| chr3 | 138665151 | 138665396 | 0.602 | FOXL2 |
| chr3 | 138665170 | 138665409 | 0.613 | FOXL2 |
| chr3 | 178916725 | 178916970 | 0.362 | PIK3CA |
| chr3 | 178916766 | 178917010 | 0.351 | PIK3CA |
| chr3 | 178916782 | 178917028 | 0.356 | PIK3CA |
| chr3 | 178916822 | 178917068 | 0.336 | PIK3CA |
| chr3 | 178917417 | 178917566 | 0.320 | PIK3CA |
| chr3 | 178917417 | 178917618 | 0.347 | PIK3CA |
| chr3 | 178917420 | 178917590 | 0.345 | PIK3CA |
| chr3 | 178917420 | 178917604 | 0.351 | PIK3CA |
| chr3 | 178921331 | 178921578 | 0.367 | PIK3CA |
| chr3 | 178921339 | 178921585 | 0.372 | PIK3CA |
| chr3 | 178921347 | 178921596 | 0.368 | PIK3CA |
| chr3 | 178921364 | 178921608 | 0.351 | PIK3CA |
| chr3 | 178927888 | 178928114 | 0.339 | PIK3CA |
| chr3 | 178927898 | 178928114 | 0.346 | PIK3CA |
| chr3 | 178927910 | 178928144 | 0.323 | PIK3CA |
| chr3 | 178927974 | 178928182 | 0.354 | PIK3CA |
| chr3 | 178935873 | 178936102 | 0.313 | PIK3CA |
| chr3 | 178935930 | 178936133 | 0.353 | PIK3CA |
| chr3 | 178935944 | 178936197 | 0.335 | PIK3CA |
| chr3 | 178935995 | 178936197 | 0.365 | PIK3CA |
| chr3 | 178936030 | 178936279 | 0.320 | PIK3CA |
| chr3 | 178951914 | 178952159 | 0.382 | PIK3CA |
| chr3 | 178951915 | 178952140 | 0.385 | PIK3CA |
| chr3 | 178951921 | 178952140 | 0.386 | PIK3CA |
| chr3 | 178951921 | 178952159 | 0.381 | PIK3CA |
| chr3 | 178951948 | 178952109 | 0.389 | PIK3CA |
| chr3 | 178952004 | 178952152 | 0.389 | PIK3CA |
| chr3 | 178952010 | 178952109 | 0.390 | PIK3CA |
| chr3 | 178952109 | 178952152 | 0.386 | PIK3CA |
| chr4 | 55151865 | 55152127 | 0.513 | PDGFRA |
| chr4 | 55593487 | 55593733 | 0.389 | KIT |
| chr4 | 55593505 | 55593749 | 0.400 | KIT |
| chr4 | 55593514 | 55593765 | 0.397 | KIT |
| chr4 | 55593530 | 55593778 | 0.390 | KIT |
| chr4 | 55594116 | 55594365 | 0.416 | KIT |
| chr4 | 55594125 | 55594365 | 0.411 | KIT |
| chr4 | 55594139 | 55594380 | 0.401 | KIT |
| chr4 | 55594139 | 55594385 | 0.401 | KIT |
| chr4 | 55599168 | 55599408 | 0.365 | KIT |
| chr4 | 55599173 | 55599417 | 0.367 | KIT |
| chr4 | 55599207 | 55599435 | 0.384 | KIT |
| chr4 | 55602645 | 55602892 | 0.423 | KIT |
| chr4 | 55602657 | 55602888 | 0.422 | KIT |
| chr4 | 55602657 | 55602892 | 0.424 | KIT |
| chr4 | 153244069 | 153244316 | 0.464 | FBXW7 |
| chr4 | 153244075 | 153244315 | 0.465 | FBXW7 |
| chr4 | 153244085 | 153244324 | 0.454 | FBXW7 |
| chr4 | 153244106 | 153244353 | 0.427 | FBXW7 |
| chr4 | 153245291 | 153245531 | 0.398 | FBXW7 |
| chr4 | 153245297 | 153245541 | 0.400 | FBXW7 |
| chr4 | 153245312 | 153245560 | 0.406 | FBXW7 |
| chr4 | 153245328 | 153245572 | 0.396 | FBXW7 |
| chr4 | 153247109 | 153247353 | 0.433 | FBXW7 |
| chr4 | 153247122 | 153247369 | 0.448 | FBXW7 |
| chr4 | 153247150 | 153247399 | 0.456 | FBXW7 |
| chr4 | 153249264 | 153249512 | 0.414 | FBXW7 |
| chr4 | 153249285 | 153249525 | 0.419 | FBXW7 |
| chr4 | 153249290 | 153249529 | 0.417 | FBXW7 |
| chr4 | 153249303 | 153249551 | 0.414 | FBXW7 |
| chr4 | 153250763 | 153251006 | 0.340 | FBXW7 |
| chr4 | 153250791 | 153251017 | 0.348 | FBXW7 |
| chr4 | 153250791 | 153251031 | 0.349 | FBXW7 |
| chr4 | 153250808 | 153251047 | 0.342 | FBXW7 |
| chr4 | 153251774 | 153252018 | 0.380 | FBXW7 |
| chr4 | 153251789 | 153252028 | 0.388 | FBXW7 |
| chr4 | 153251792 | 153252040 | 0.382 | FBXW7 |
| chr4 | 153251832 | 153252071 | 0.379 | FBXW7 |
| chr4 | 153258843 | 153259092 | 0.356 | FBXW7 |
| chr4 | 153258850 | 153259097 | 0.355 | FBXW7 |
| chr4 | 153258888 | 153259137 | 0.344 | FBXW7 |
| chr4 | 153258953 | 153259199 | 0.368 | FBXW7 |
| chr4 | 153268077 | 153268219 | 0.517 | FBXW7 |
| chr4 | 153268078 | 153268219 | 0.514 | FBXW7 |
| chr4 | 153268079 | 153268219 | 0.518 | FBXW7 |
| chr5 | 56161075 | 56161306 | 0.328 | MAP3K1 |
| chr5 | 56161096 | 56161325 | 0.330 | MAP3K1 |
| chr5 | 56161113 | 56161321 | 0.349 | MAP3K1 |
| chr5 | 56161178 | 56161386 | 0.321 | MAP3K1 |
| chr5 | 56161545 | 56161787 | 0.346 | MAP3K1 |
| chr5 | 56161548 | 56161797 | 0.348 | MAP3K1 |
| chr5 | 56161563 | 56161807 | 0.355 | MAP3K1 |
| chr5 | 56161577 | 56161806 | 0.365 | MAP3K1 |
| chr5 | 56180437 | 56180676 | 0.363 | MAP3K1 |
| chr5 | 56180457 | 56180659 | 0.389 | MAP3K1 |
| chr5 | 56180502 | 56180707 | 0.369 | MAP3K1 |
| chr5 | 56181614 | 56181861 | 0.335 | MAP3K1 |
| chr5 | 56181627 | 56181876 | 0.332 | MAP3K1 |
| chr5 | 56181632 | 56181871 | 0.338 | MAP3K1 |
| chr5 | 56181648 | 56181887 | 0.346 | MAP3K1 |
| chr5 | 56183135 | 56183374 | 0.429 | MAP3K1 |
| chr5 | 56183138 | 56183347 | 0.429 | MAP3K1 |
| chr5 | 56183138 | 56183361 | 0.429 | MAP3K1 |
| chr5 | 56183176 | 56183375 | 0.455 | MAP3K1 |
| chr5 | 112043137 | 112043364 | 0.649 | APC |
| chr5 | 112043137 | 112043365 | 0.651 | APC |
| chr5 | 112043186 | 112043431 | 0.663 | APC |
| chr5 | 112043190 | 112043431 | 0.661 | APC |
| chr5 | 112043201 | 112043471 | 0.672 | APC |
| chr5 | 112043206 | 112043428 | 0.668 | APC |
| chr5 | 112073451 | 112073699 | 0.639 | APC |
| chr5 | 112073948 | 112074148 | 0.488 | APC |
| chr5 | 112090491 | 112090691 | 0.373 | APC |
| chr5 | 112102006 | 112102208 | 0.330 | APC |
| chr5 | 112102893 | 112103093 | 0.433 | APC |
| chr5 | 112111368 | 112111578 | 0.289 | APC |
| chr5 | 112116375 | 112116639 | 0.340 | APC |
| chr5 | 112128134 | 112128341 | 0.332 | APC |
| chr5 | 112128152 | 112128420 | 0.309 | APC |
| chr5 | 112136780 | 112137014 | 0.404 | APC |
| chr5 | 112151086 | 112151286 | 0.368 | APC |
| chr5 | 112154610 | 112154859 | 0.424 | APC |
| chr5 | 112154826 | 112155071 | 0.504 | APC |
| chr5 | 112157485 | 112157695 | 0.341 | APC |
| chr5 | 112162781 | 112162991 | 0.379 | APC |
| chr5 | 112163441 | 112163655 | 0.288 | APC |
| chr5 | 112163452 | 112163681 | 0.309 | APC |
| chr5 | 112164471 | 112164655 | 0.319 | APC |
| chr5 | 112170600 | 112170800 | 0.393 | APC |
| chr5 | 112170810 | 112171010 | 0.289 | APC |
| chr5 | 112173249 | 112173449 | 0.383 | APC |
| chr5 | 112173471 | 112173712 | 0.405 | APC |
| chr5 | 112173688 | 112173932 | 0.404 | APC |
| chr5 | 112173840 | 112174071 | 0.466 | APC |
| chr5 | 112174070 | 112174313 | 0.361 | APC |
| chr5 | 112174297 | 112174537 | 0.353 | APC |
| chr5 | 112174544 | 112174783 | 0.392 | APC |
| chr5 | 112174788 | 112175027 | 0.400 | APC |
| chr5 | 112174985 | 112175185 | 0.378 | APC |
| chr5 | 112175164 | 112175405 | 0.459 | APC |
| chr5 | 112175413 | 112175652 | 0.471 | APC |
| chr5 | 112175519 | 112175768 | 0.460 | APC |
| chr5 | 112175523 | 112175780 | 0.461 | APC |
| chr5 | 112175853 | 112176053 | 0.333 | APC |
| chr5 | 112175867 | 112176116 | 0.380 | APC |
| chr5 | 112175900 | 112176040 | 0.326 | APC |
| chr5 | 112176145 | 112176391 | 0.449 | APC |
| chr5 | 112176384 | 112176633 | 0.412 | APC |
| chr5 | 112176631 | 112176875 | 0.322 | APC |
| chr5 | 112176860 | 112177104 | 0.396 | APC |
| chr5 | 112176995 | 112177234 | 0.400 | APC |
| chr5 | 112177404 | 112177650 | 0.385 | APC |
| chr5 | 112177589 | 112177789 | 0.408 | APC |
| chr5 | 112177796 | 112178080 | 0.389 | APC |
| chr5 | 112178130 | 112178379 | 0.456 | APC |
| chr5 | 112178293 | 112178573 | 0.391 | APC |
| chr5 | 112178510 | 112178755 | 0.407 | APC |
| chr5 | 112178763 | 112179036 | 0.449 | APC |
| chr5 | 112179030 | 112179324 | 0.380 | APC |
| chr5 | 112179325 | 112179525 | 0.423 | APC |
| chr5 | 112179582 | 112179827 | 0.472 | APC |
| chr5 | 112180193 | 112180393 | 0.338 | APC |
| chr5 | 112181061 | 112181261 | 0.398 | APC |
| chr5 | 112181929 | 112182129 | 0.313 | APC |
| chr5 | 131892606 | 131892857 | 0.718 | RAD50 |
| chr5 | 131892983 | 131893197 | 0.474 | RAD50 |
| chr5 | 131892984 | 131893223 | 0.463 | RAD50 |
| chr5 | 131893020 | 131893223 | 0.466 | RAD50 |
| chr5 | 131893049 | 131893292 | 0.492 | RAD50 |
| chr5 | 131894808 | 131895037 | 0.252 | RAD50 |
| chr5 | 131911382 | 131911618 | 0.376 | RAD50 |
| chr5 | 131915004 | 131915223 | 0.382 | RAD50 |
| chr5 | 131915567 | 131915717 | 0.364 | RAD50 |
| chr5 | 131923251 | 131923500 | 0.312 | RAD50 |
| chr5 | 131923517 | 131923757 | 0.328 | RAD50 |
| chr5 | 131924402 | 131924651 | 0.364 | RAD50 |
| chr5 | 131925323 | 131925552 | 0.343 | RAD50 |
| chr5 | 131926850 | 131927094 | 0.363 | RAD50 |
| chr5 | 131927570 | 131927750 | 0.354 | RAD50 |
| chr5 | 131930491 | 131930738 | 0.323 | RAD50 |
| chr5 | 131931278 | 131931508 | 0.442 | RAD50 |
| chr5 | 131939061 | 131939291 | 0.377 | RAD50 |
| chr5 | 131939553 | 131939789 | 0.350 | RAD50 |
| chr5 | 131940409 | 131940654 | 0.346 | RAD50 |
| chr5 | 131944351 | 131944557 | 0.261 | RAD50 |
| chr5 | 131944387 | 131944597 | 0.251 | RAD50 |
| chr5 | 131944873 | 131945090 | 0.271 | RAD50 |
| chr5 | 131951603 | 131951803 | 0.323 | RAD50 |
| chr5 | 131951820 | 131952020 | 0.294 | RAD50 |
| chr5 | 131953730 | 131953966 | 0.333 | RAD50 |
| chr5 | 131972864 | 131973090 | 0.454 | RAD50 |
| chr5 | 131973728 | 131973957 | 0.443 | RAD50 |
| chr5 | 131976168 | 131976417 | 0.520 | RAD50 |
| chr5 | 131977815 | 131978052 | 0.378 | RAD50 |
| chr5 | 131978075 | 131978308 | 0.372 | RAD50 |
| chr5 | 131978265 | 131978518 | 0.417 | RAD50 |
| chr5 | 131978882 | 131979101 | 0.486 | RAD50 |
| chr5 | 170837389 | 170837646 | 0.318 | NPM1 |
| chr5 | 170837423 | 170837672 | 0.316 | NPM1 |
| chr6 | 35419950 | 35420204 | 0.710 | FANCE |
| chr6 | 35420484 | 35420712 | 0.677 | FANCE |
| chr6 | 35423523 | 35423753 | 0.610 | FANCE |
| chr6 | 35423747 | 35423980 | 0.568 | FANCE |
| chr6 | 35423937 | 35424160 | 0.500 | FANCE |
| chr6 | 35425325 | 35425579 | 0.541 | FANCE |
| chr6 | 35425576 | 35425862 | 0.516 | FANCE |
| chr6 | 35425990 | 35426190 | 0.602 | FANCE |
| chr6 | 35427014 | 35427214 | 0.443 | FANCE |
| chr6 | 35427343 | 35427543 | 0.552 | FANCE |
| chr6 | 35428293 | 35428492 | 0.560 | FANCE |
| chr6 | 35433986 | 35434203 | 0.550 | FANCE |
| chr6 | 35434173 | 35434385 | 0.493 | FANCE |
| chr6 | 43544117 | 43544321 | 0.459 | POLH |
| chr6 | 43550014 | 43550163 | 0.420 | POLH |
| chr6 | 43550754 | 43550953 | 0.415 | POLH |
| chr6 | 43554978 | 43555223 | 0.476 | POLH |
| chr6 | 43565476 | 43565625 | 0.467 | POLH |
| chr6 | 43568621 | 43568821 | 0.448 | POLH |
| chr6 | 43571533 | 43571733 | 0.423 | POLH |
| chr6 | 43572382 | 43572582 | 0.433 | POLH |
| chr6 | 43572829 | 43573058 | 0.374 | POLH |
| chr6 | 43578220 | 43578420 | 0.507 | POLH |
| chr6 | 43581333 | 43581514 | 0.407 | POLH |
| chr6 | 43581613 | 43581843 | 0.433 | POLH |
| chr6 | 43581852 | 43582085 | 0.517 | POLH |
| chr6 | 43582072 | 43582231 | 0.475 | POLH |
| chr6 | 43587356 | 43587593 | 0.361 | POLH |
| chr6 | 43587578 | 43587778 | 0.393 | POLH |
| chr6 | 43587842 | 43588084 | 0.444 | POLH |
| chr6 | 152265305 | 152265551 | 0.555 | ESR1 |
| chr6 | 152265322 | 152265569 | 0.556 | ESR1 |
| chr6 | 152265335 | 152265582 | 0.548 | ESR1 |
| chr6 | 152265349 | 152265597 | 0.562 | ESR1 |
| chr6 | 152332663 | 152332910 | 0.444 | ESR1 |
| chr6 | 152332707 | 152332937 | 0.463 | ESR1 |
| chr6 | 152332732 | 152332961 | 0.483 | ESR1 |
| chr6 | 152415449 | 152415694 | 0.553 | ESR1 |
| chr6 | 152415449 | 152415729 | 0.555 | ESR1 |
| chr6 | 152415452 | 152415694 | 0.556 | ESR1 |
| chr6 | 152415469 | 152415763 | 0.542 | ESR1 |
| chr6 | 152419792 | 152420036 | 0.576 | ESR1 |
| chr6 | 152419822 | 152420047 | 0.575 | ESR1 |
| chr6 | 152419877 | 152420111 | 0.591 | ESR1 |
| chr7 | 6029374 | 6029583 | 0.367 | PMS2 |
| chr7 | 6035119 | 6035348 | 0.387 | PMS2 |
| chr7 | 6036912 | 6037112 | 0.378 | PMS2 |
| chr7 | 6038718 | 6039008 | 0.430 | PMS2 |
| chr7 | 55241468 | 55241710 | 0.564 | EGFR |
| chr7 | 55241542 | 55241789 | 0.565 | EGFR |
| chr7 | 55241576 | 55241746 | 0.526 | EGFR |
| chr7 | 55241613 | 55241853 | 0.531 | EGFR |
| chr7 | 55242269 | 55242500 | 0.509 | EGFR |
| chr7 | 55242271 | 55242505 | 0.506 | EGFR |
| chr7 | 55242272 | 55242520 | 0.506 | EGFR |
| chr7 | 55242280 | 55242532 | 0.506 | EGFR |
| chr7 | 55242319 | 55242558 | 0.496 | EGFR |
| chr7 | 55242332 | 55242584 | 0.490 | EGFR |
| chr7 | 55248852 | 55249080 | 0.585 | EGFR |
| chr7 | 55248896 | 55249120 | 0.622 | EGFR |
| chr7 | 55248933 | 55249182 | 0.600 | EGFR |
| chr7 | 55248937 | 55249187 | 0.598 | EGFR |
| chr7 | 55248961 | 55249208 | 0.597 | EGFR |
| chr7 | 55259335 | 55259571 | 0.523 | EGFR |
| chr7 | 55259337 | 55259586 | 0.532 | EGFR |
| chr7 | 55259356 | 55259602 | 0.534 | EGFR |
| chr7 | 55259368 | 55259570 | 0.537 | EGFR |
| chr7 | 55259391 | 55259640 | 0.544 | EGFR |
| chr7 | 116411722 | 116411963 | 0.417 | MET |
| chr7 | 116411832 | 116412076 | 0.424 | MET |
| chr7 | 116411854 | 116412102 | 0.410 | MET |
| chr7 | 116411942 | 116412183 | 0.347 | MET |
| chr7 | 140453006 | 140453257 | 0.381 | BRAF |
| chr7 | 140453042 | 140453283 | 0.364 | BRAF |
| chr7 | 140453060 | 140453259 | 0.385 | BRAF |
| chr7 | 140453085 | 140453319 | 0.336 | BRAF |
| chr7 | 140453086 | 140453335 | 0.340 | BRAF |
| chr7 | 140453105 | 140453353 | 0.329 | BRAF |
| chr7 | 140481224 | 140481471 | 0.355 | BRAF |
| chr7 | 140481234 | 140481475 | 0.360 | BRAF |
| chr7 | 140481251 | 140481496 | 0.358 | BRAF |
| chr7 | 140481263 | 140481507 | 0.363 | BRAF |
| chr8 | 90947700 | 90947947 | 0.319 | NBN |
| chr8 | 90949123 | 90949343 | 0.326 | NBN |
| chr8 | 90955446 | 90955690 | 0.351 | NBN |
| chr8 | 90958290 | 90958510 | 0.321 | NBN |
| chr8 | 90959971 | 90960119 | 0.329 | NBN |
| chr8 | 90965407 | 90965703 | 0.347 | NBN |
| chr8 | 90965709 | 90965960 | 0.333 | NBN |
| chr8 | 90967522 | 90967722 | 0.388 | NBN |
| chr8 | 90967762 | 90967962 | 0.299 | NBN |
| chr8 | 90970951 | 90971177 | 0.414 | NBN |
| chr8 | 90976592 | 90976816 | 0.324 | NBN |
| chr8 | 90976592 | 90976817 | 0.323 | NBN |
| chr8 | 90982598 | 90982805 | 0.385 | NBN |
| chr8 | 90983304 | 90983514 | 0.308 | NBN |
| chr8 | 90990344 | 90990544 | 0.318 | NBN |
| chr8 | 90992886 | 90993086 | 0.323 | NBN |
| chr8 | 90993103 | 90993303 | 0.284 | NBN |
| chr8 | 90993522 | 90993742 | 0.321 | NBN |
| chr8 | 90994780 | 90995004 | 0.347 | NBN |
| chr8 | 90996593 | 90996859 | 0.678 | NBN |
| chr9 | 5073651 | 5073873 | 0.354 | JAK2 |
| chr9 | 21968200 | 21968432 | 0.545 | CDKN2A |
| chr9 | 21970919 | 21971216 | 0.721 | CDKN2A |
| chr9 | 21970920 | 21971216 | 0.721 | CDKN2A |
| chr9 | 21973486 | 21973718 | 0.365 | CDKN2A |
| chr9 | 21974363 | 21974602 | 0.500 | CDKN2A |
| chr9 | 21994083 | 21994336 | 0.654 | CDKN2A |
| chr9 | 21994085 | 21994334 | 0.656 | CDKN2A |
| chr9 | 35073773 | 35074045 | 0.432 | FANCG |
| chr9 | 35074052 | 35074258 | 0.517 | FANCG |
| chr9 | 35074248 | 35074484 | 0.565 | FANCG |
| chr9 | 35074847 | 35075072 | 0.535 | FANCG |
| chr9 | 35075125 | 35075335 | 0.493 | FANCG |
| chr9 | 35075354 | 35075564 | 0.531 | FANCG |
| chr9 | 35075451 | 35075651 | 0.562 | FANCG |
| chr9 | 35075668 | 35075868 | 0.587 | FANCG |
| chr9 | 35075897 | 35076097 | 0.557 | FANCG |
| chr9 | 35076388 | 35076632 | 0.494 | FANCG |
| chr9 | 35076699 | 35076959 | 0.529 | FANCG |
| chr9 | 35076908 | 35077108 | 0.537 | FANCG |
| chr9 | 35077204 | 35077404 | 0.517 | FANCG |
| chr9 | 35078145 | 35078385 | 0.593 | FANCG |
| chr9 | 35078512 | 35078712 | 0.522 | FANCG |
| chr9 | 35079068 | 35079268 | 0.587 | FANCG |
| chr9 | 35079437 | 35079667 | 0.671 | FANCG |
| chr9 | 35079767 | 35079970 | 0.667 | FANCG |
| chr9 | 35079828 | 35080069 | 0.702 | FANCG |
| chr9 | 35079839 | 35080069 | 0.706 | FANCG |
| chr9 | 35079923 | 35080159 | 0.667 | FANCG |
| chr9 | 80336120 | 80336372 | 0.478 | GNAQ |
| chr9 | 80336121 | 80336372 | 0.476 | GNAQ |
| chr9 | 80336223 | 80336472 | 0.500 | GNAQ |
| chr9 | 80336253 | 80336500 | 0.484 | GNAQ |
| chr9 | 80336259 | 80336500 | 0.483 | GNAQ |
| chr9 | 80409367 | 80409598 | 0.353 | GNAQ |
| chr9 | 80409379 | 80409628 | 0.352 | GNAQ |
| chr9 | 97863312 | 97863512 | 0.572 | FANCC |
| chr9 | 97863840 | 97864111 | 0.548 | FANCC |
| chr9 | 97869338 | 97869594 | 0.595 | FANCC |
| chr9 | 97872619 | 97872829 | 0.308 | FANCC |
| chr9 | 97872955 | 97873165 | 0.398 | FANCC |
| chr9 | 97873179 | 97873409 | 0.494 | FANCC |
| chr9 | 97873711 | 97873957 | 0.587 | FANCC |
| chr9 | 97876827 | 97877027 | 0.517 | FANCC |
| chr9 | 97879613 | 97879852 | 0.404 | FANCC |
| chr9 | 97887262 | 97887462 | 0.403 | FANCC |
| chr9 | 97888682 | 97888882 | 0.423 | FANCC |
| chr9 | 97897551 | 97897761 | 0.417 | FANCC |
| chr9 | 97897761 | 97897961 | 0.313 | FANCC |
| chr9 | 97912208 | 97912455 | 0.456 | FANCC |
| chr9 | 97933298 | 97933498 | 0.363 | FANCC |
| chr9 | 97934219 | 97934419 | 0.333 | FANCC |
| chr9 | 98002823 | 98003043 | 0.321 | FANCC |
| chr9 | 98009694 | 98009940 | 0.328 | FANCC |
| chr9 | 98011375 | 98011575 | 0.418 | FANCC |
| chr9 | 98011585 | 98011785 | 0.433 | FANCC |
| chr9 | 98079868 | 98080148 | 0.669 | FANCC |
| chr9 | 100437190 | 100437390 | 0.428 | XPA |
| chr9 | 100437359 | 100437558 | 0.475 | XPA |
| chr9 | 100437525 | 100437765 | 0.299 | XPA |
| chr9 | 100437793 | 100437993 | 0.383 | XPA |
| chr9 | 100444474 | 100444677 | 0.397 | XPA |
| chr9 | 100447108 | 100447308 | 0.348 | XPA |
| chr9 | 100449321 | 100449540 | 0.309 | XPA |
| chr9 | 100451744 | 100451944 | 0.323 | XPA |
| chr9 | 100455831 | 100456031 | 0.328 | XPA |
| chr9 | 100459275 | 100459499 | 0.720 | XPA |
| chr9 | 100459396 | 100459601 | 0.728 | XPA |
| chr9 | 100459482 | 100459691 | 0.724 | XPA |
| chr9 | 100459482 | 100459695 | 0.720 | XPA |
| chr9 | 100459482 | 100459721 | 0.721 | XPA |
| chrX | 14861796 | 14861980 | 0.416 | FANCB |
| chrX | 14861961 | 14862165 | 0.322 | FANCB |
| chrX | 14862588 | 14862788 | 0.368 | FANCB |
| chrX | 14862805 | 14863005 | 0.294 | FANCB |
| chrX | 14862977 | 14863177 | 0.358 | FANCB |
| chrX | 14863227 | 14863447 | 0.403 | FANCB |
| chrX | 14868691 | 14868896 | 0.306 | FANCB |
| chrX | 14871130 | 14871330 | 0.313 | FANCB |
| chrX | 14875743 | 14875993 | 0.307 | FANCB |
| chrX | 14877225 | 14877425 | 0.313 | FANCB |
| chrX | 14877435 | 14877635 | 0.274 | FANCB |
| chrX | 14882681 | 14882881 | 0.393 | FANCB |
| chrX | 14882885 | 14883085 | 0.328 | FANCB |
| chrX | 14883089 | 14883289 | 0.383 | FANCB |
| chrX | 14883275 | 14883501 | 0.291 | FANCB |
| chrX | 14883493 | 14883735 | 0.350 | FANCB |
| chrX | 14887070 | 14887270 | 0.323 | FANCB |
| chrX | 14890941 | 14891170 | 0.578 | FANCB |
| chrX | 47426013 | 47426262 | 0.636 | ARAF |
| chrX | 47426015 | 47426263 | 0.635 | ARAF |
| chrX | 47426016 | 47426262 | 0.640 | ARAF |
| chrX | 47426063 | 47426266 | 0.627 | ARAF |
| chrX | 66765925 | 66766173 | 0.635 | AR |
| chrX | 66765934 | 66766173 | 0.638 | AR |
| chrX | 66766002 | 66766226 | 0.671 | AR |
| chrX | 66766013 | 66766226 | 0.682 | AR |
| chrX | 66931238 | 66931486 | 0.522 | AR |
| chrX | 66931245 | 66931461 | 0.525 | AR |
| chrX | 66931245 | 66931492 | 0.524 | AR |
| chrX | 66931246 | 66931484 | 0.523 | AR |
| chrX | 66937264 | 66937498 | 0.536 | AR |
| chrX | 66937327 | 66937559 | 0.532 | AR |
| chrX | 66937327 | 66937576 | 0.524 | AR |
| chrX | 66943491 | 66943695 | 0.468 | AR |
| chrX | 66943494 | 66943696 | 0.473 | AR |
| chrX | 66943515 | 66943684 | 0.476 | AR |
| chrX | 66943534 | 66943689 | 0.474 | AR |

### DETAILED DESCRIPTION

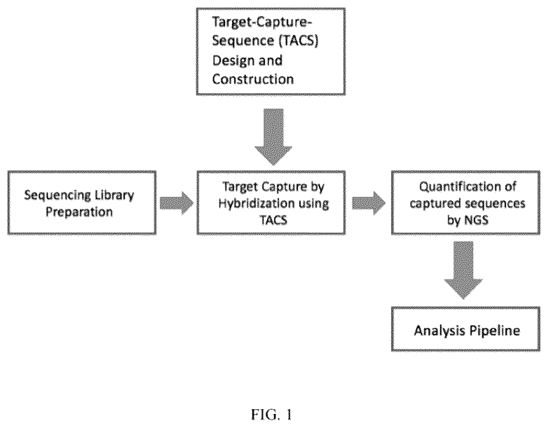

The invention pertains to a method for analyzing tumor biomarker sequences that involves hybridization-based enrichment of selected target regions across the human genome in a multiplexed panel assay, followed by quantification, coupled with a novel bioinformatics and mathematical analysis pipeline. An overview of the method is shown schematically in FIG. 1.

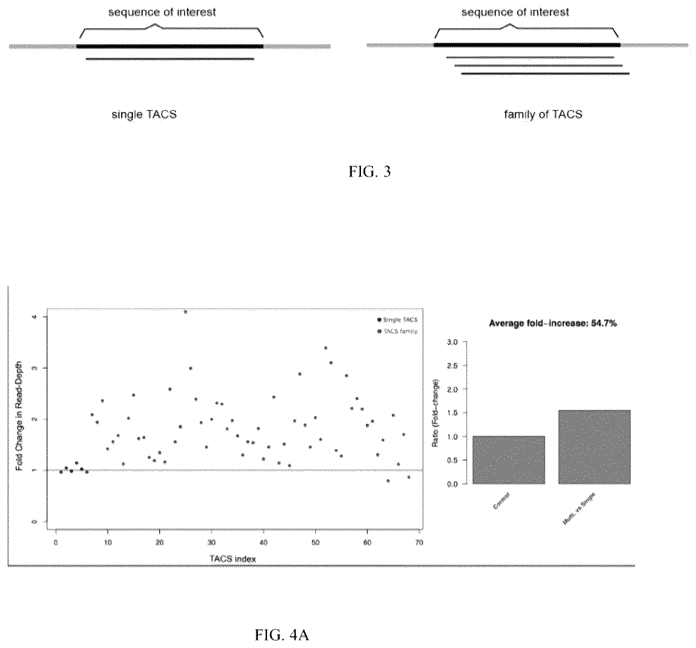

In-solution hybridization enrichment has been used in the past to enrich specific regions of interest prior to sequencing (see e.g., Meyer, M and Kirchner, M. (2010) Cold Spring Harb. Protoc. 2010 (6):pdbprot5448; Liao, G. J. et al. (2012) PLoS One 7:e38154; Maricic, T. et al. (2010) PLoS One 5:e14004; Tewhey, R. et al. (2009) Genome Biol. 10:R116; Tsangaras, K. et al. (2014) PLoS One 9:e109101; PCT Publication WO 2016/189388; US Patent Publication 2016/0340733; Koumbaris, G. et al. (2016) Clinical chemistry, 62(6), pp.848-855). However, for the methods of the invention, the target sequences (referred to as TArget Capture Sequences, or TACS) used to enrich for specific regions of interest have been optimized for maximum efficiency, specificity and accuracy and, furthermore, in certain embodiments are used in families of TACS, comprising a plurality of members that bind to the same tumor biomarker sequence but with differing start and/or stop positions, such that enrichment of the tumor biomarker sequences of interest is significantly improved compared to use of a single TACS binding to the genomic sequence. An example of a configuration of such families of TACS is illustrated schematically in FIG. 3, showing that the different start and/or stop positions of the members of the TACS family when bound to the genomic sequence of interest results in a staggered binding pattern for the family members.

The use of families of TACS with the TACS pool that bind to each target sequence of interest, as compared to use of a single TACS within the TACS pool that binds to each target sequence of interest, significantly increases enrichment for the target sequences of interest, as evidenced by a greater than 50% average increase in read-depth for the family of TACS versus a single TACS. Comparison of use of a family of TACS versus a single TACS, and the significantly improved read-depth that was observed, is described in detail in Example 5.

### Tumor Biomarker Detection

The methods and kits of the disclosure are used in the analysis of tumor biomarkers in biological samples. As described in detail in Examples 6-9, the methods of the invention can used for the detection of large panels of tumor biomarkers at tumor loads as low as 0.1% and can detect tumor biomarkers in both tumor tissue and in liquid biopsy samples from tumor patients. Accordingly, in one aspect, the invention pertains to a method of detecting one or more tumor biomarkers in a DNA sample from a subject having or suspected of having a tumor, the method comprising:
(a) preparing a sequencing library from the DNA sample;
(b) hybridizing the sequencing library to a pool of double-stranded TArget Capture Sequences (TACS) that bind to one or more tumor biomarker sequences of interest, wherein:
   (i) each member sequence within the pool of TACS is between 100-500 base pairs in length, each member sequence having a 5′ end and a 3′ end;
   (ii) preferably each member sequence binds to the tumor biomarker sequence of interest at least 50 base pairs away, on both the 5′ end and the 3′ end, from regions harboring Copy Number Variations (CNVs), Segmental duplications or repetitive DNA elements; and
   (iii) the GC content of the pool of TACS is between 19% and 80%, as determined by calculating the GC content of each member within the pool of TACS;
(c) isolating members of the sequencing library that bind to the pool of TACS to obtain an enriched library;
(d) amplifying and sequencing the enriched library; and
(e) performing statistical analysis on the enriched library sequences, optionally utilizing only fragments of a specific size range, to thereby detect the tumor biomarker(s) in the DNA sample.

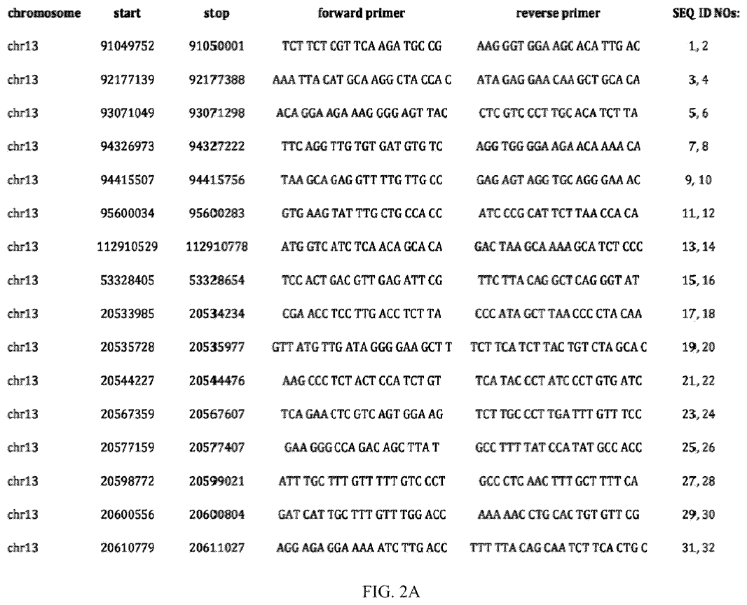

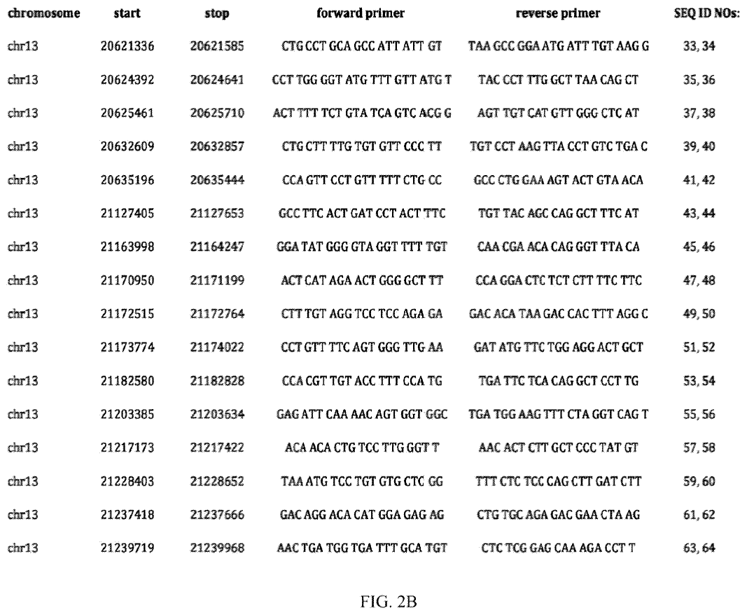

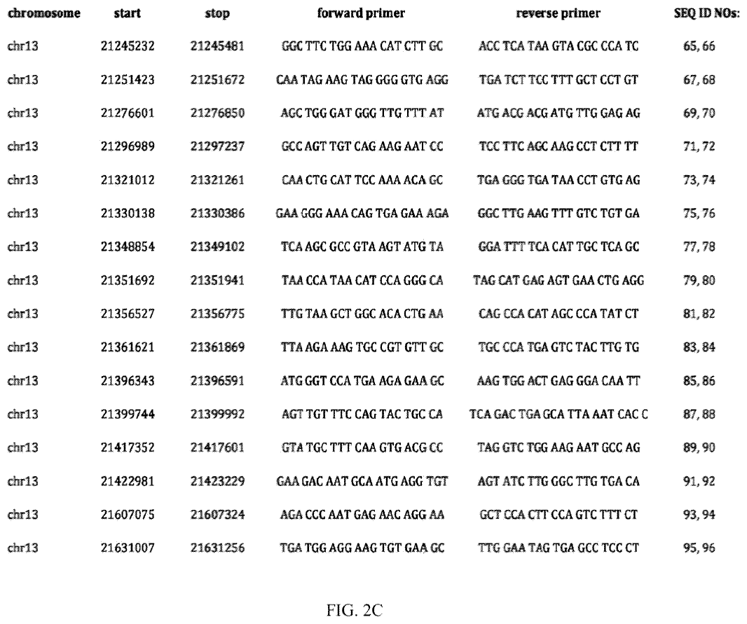

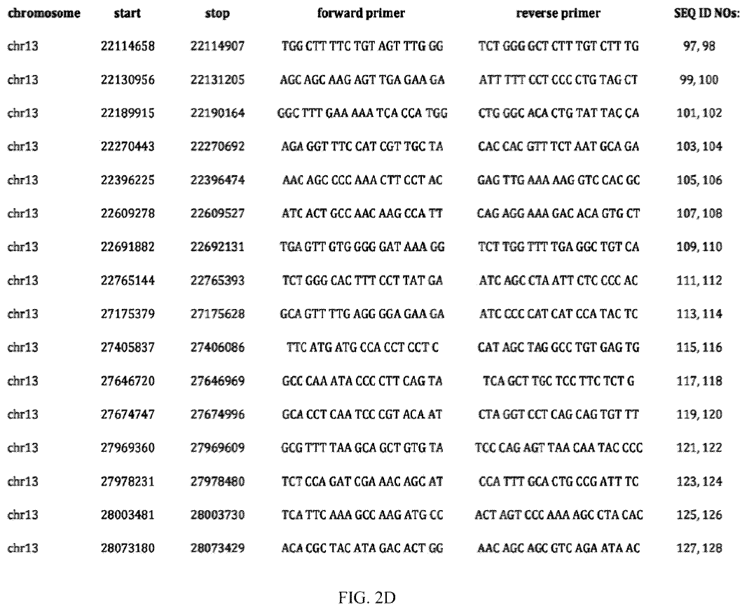

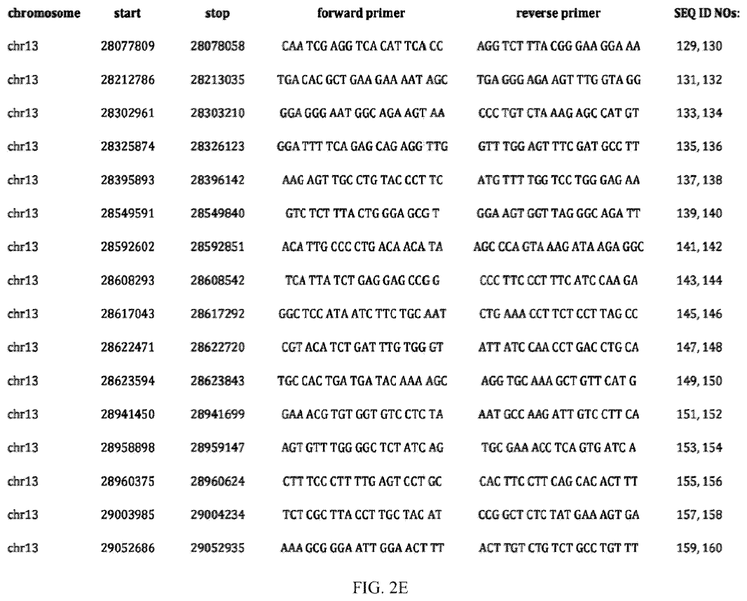

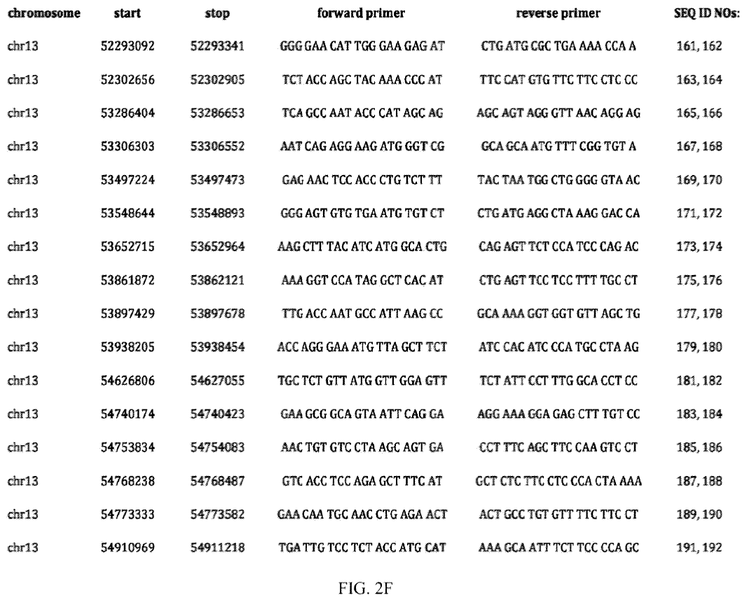

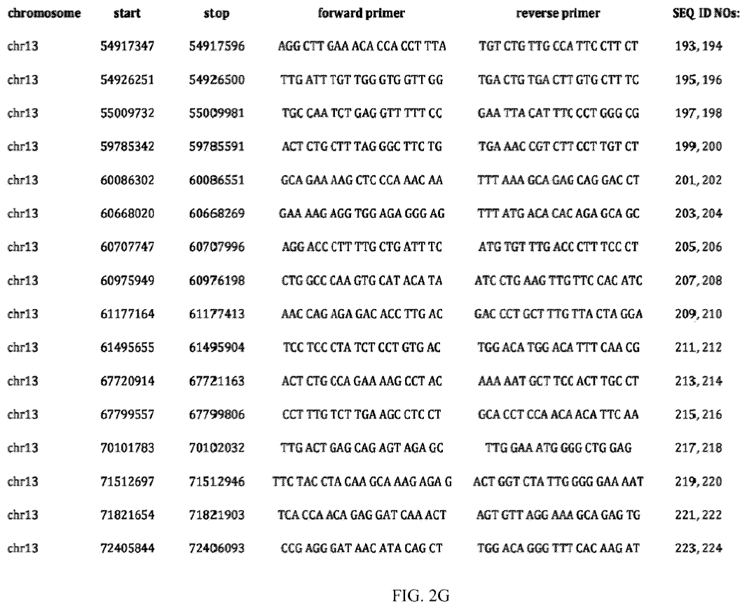

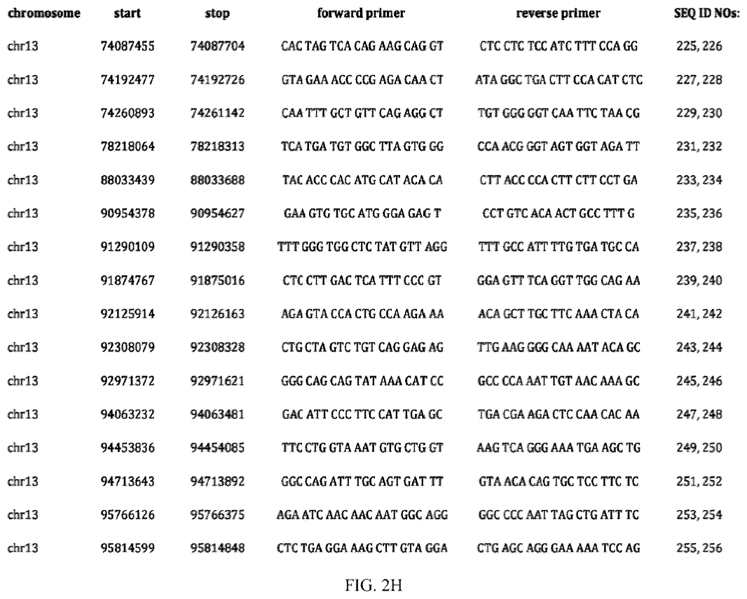

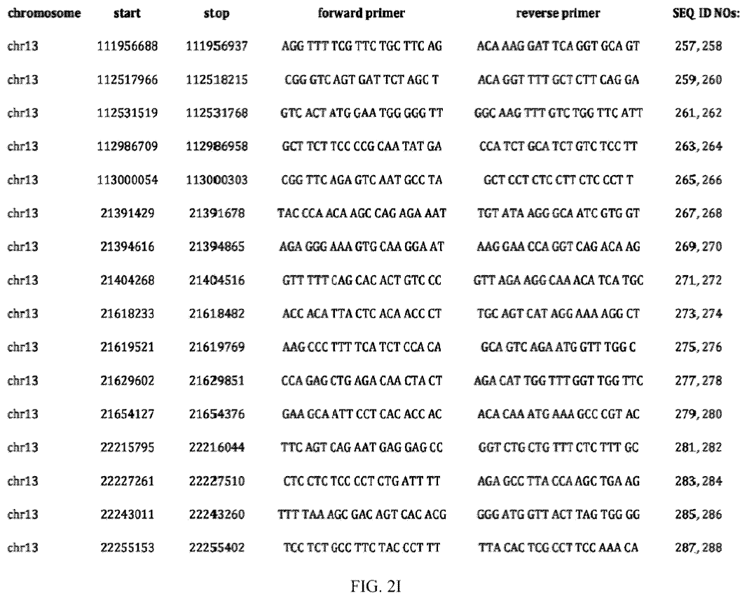

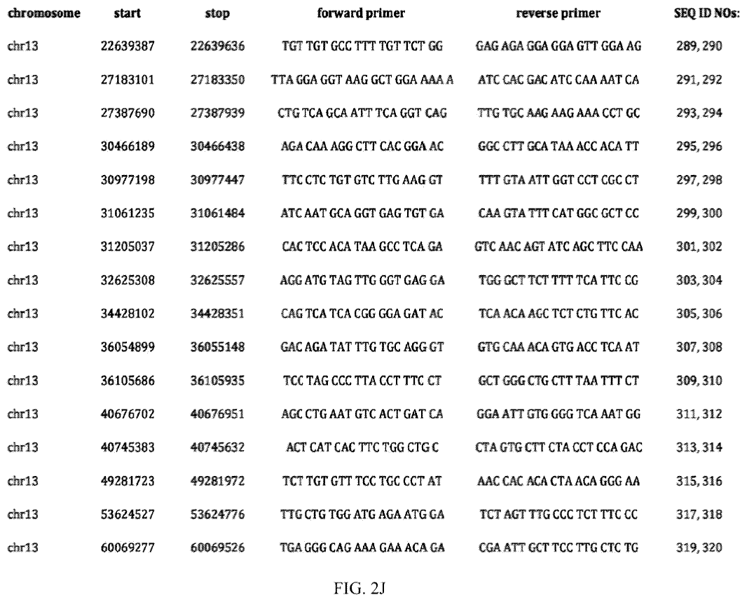

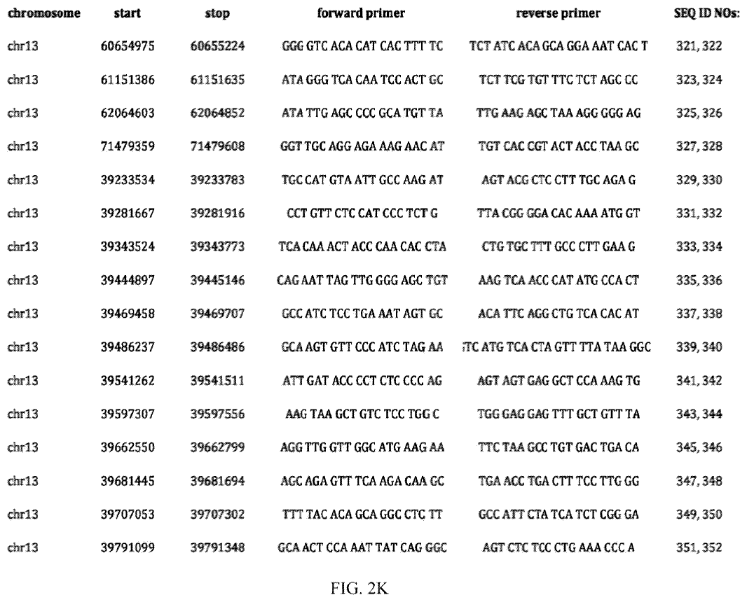

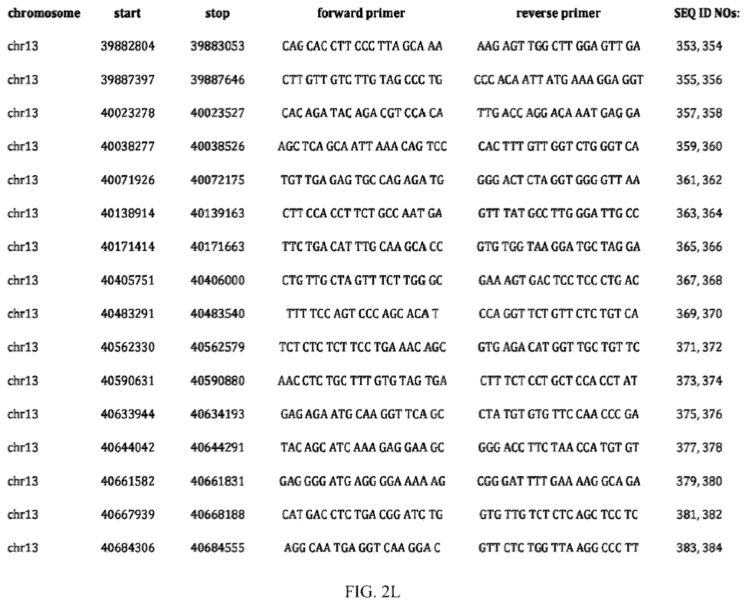

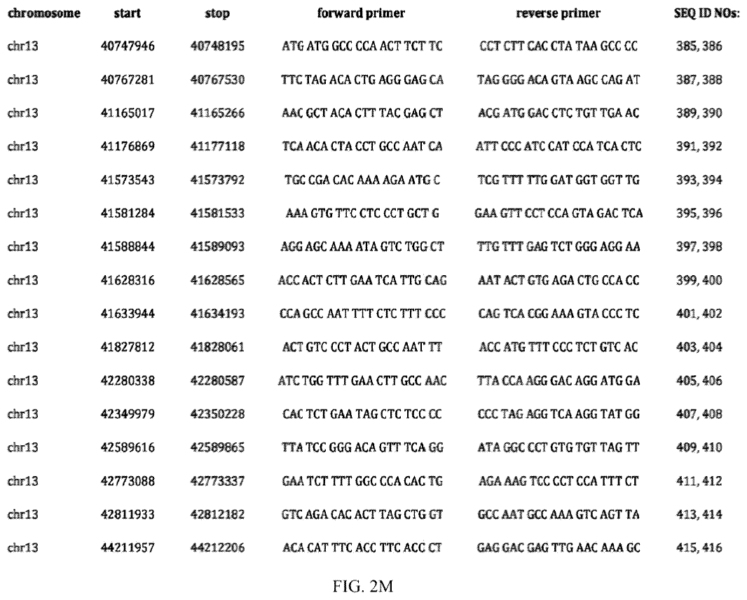

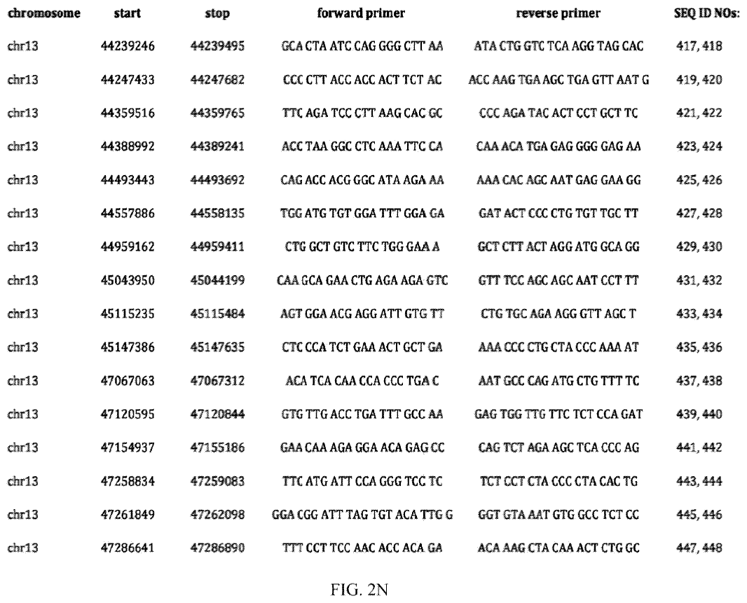

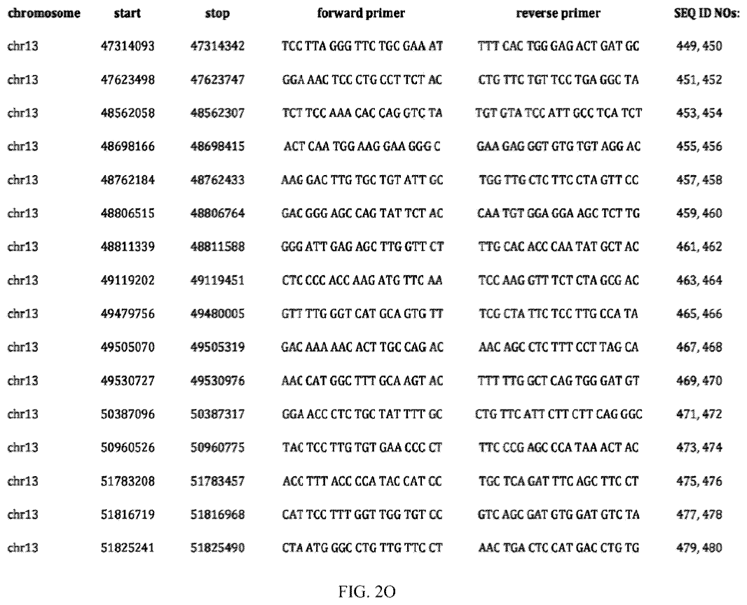

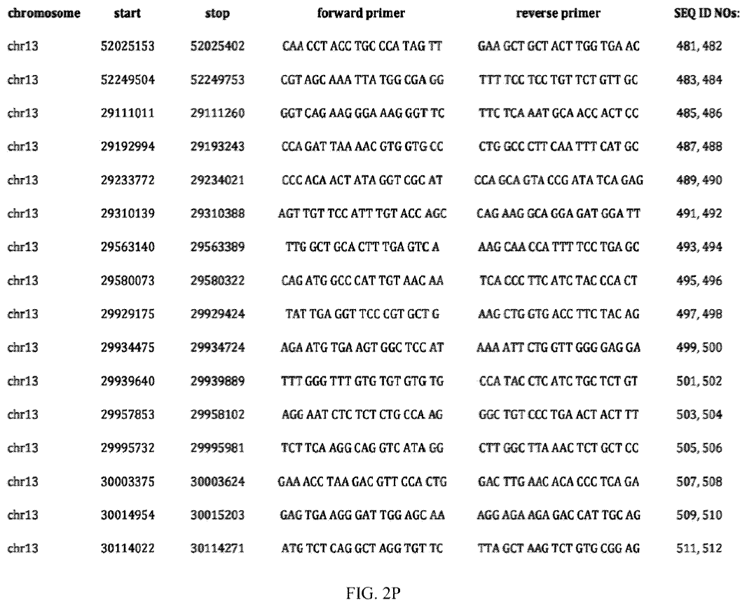

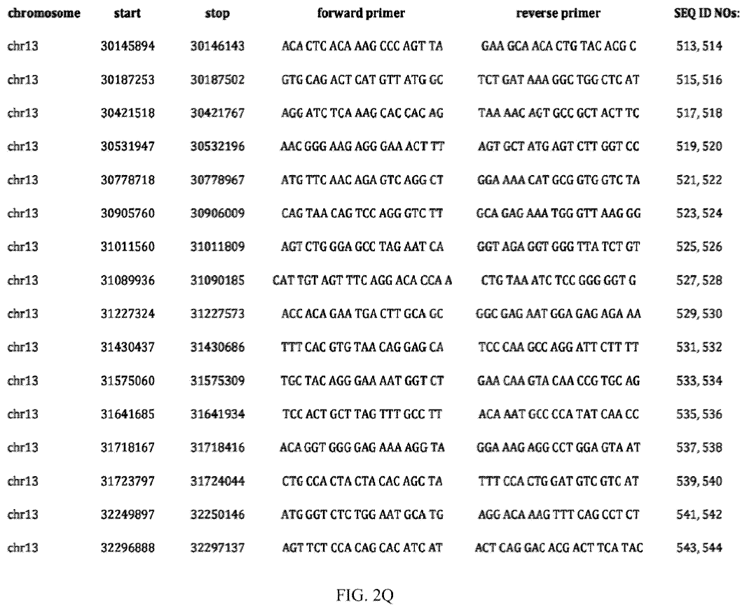

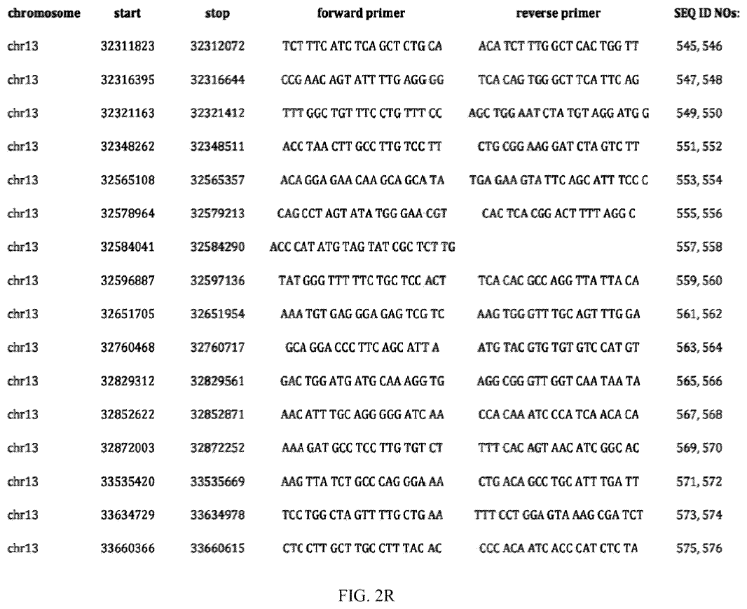

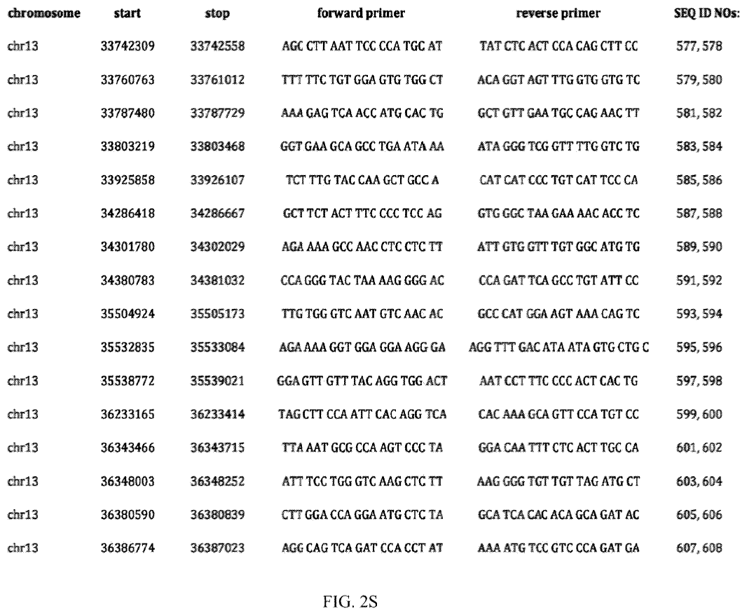

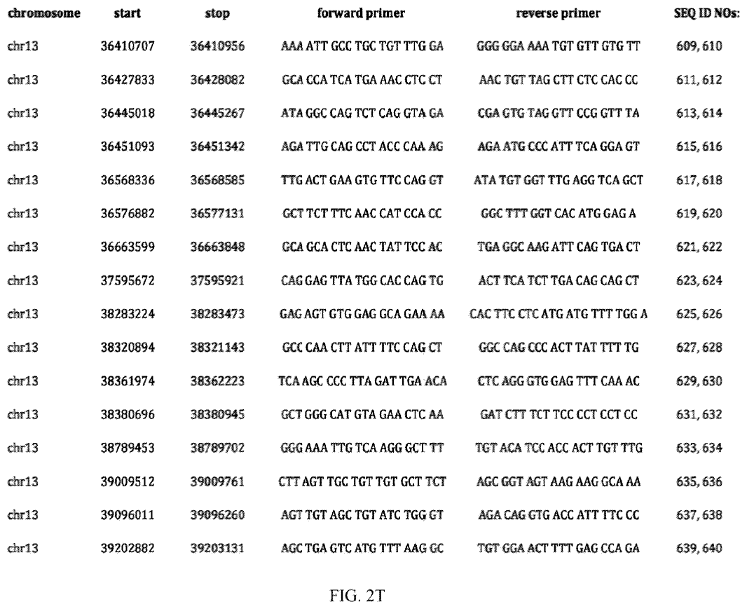

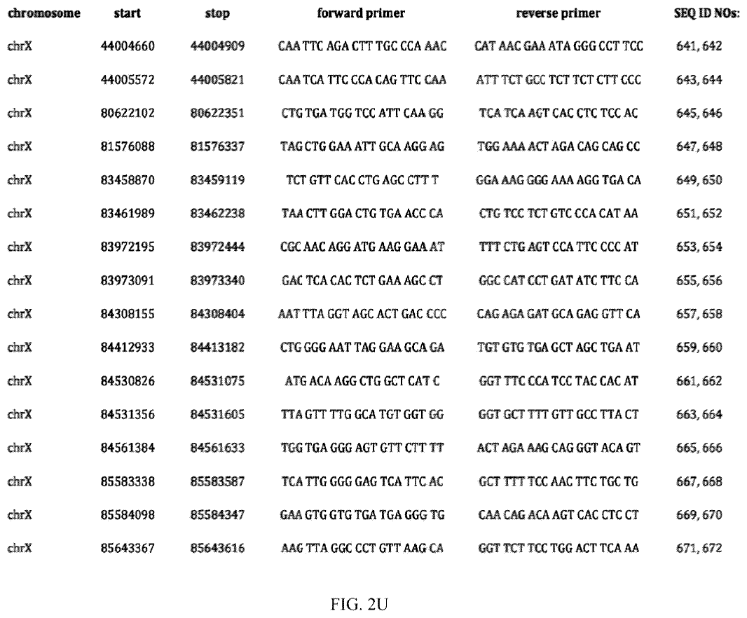

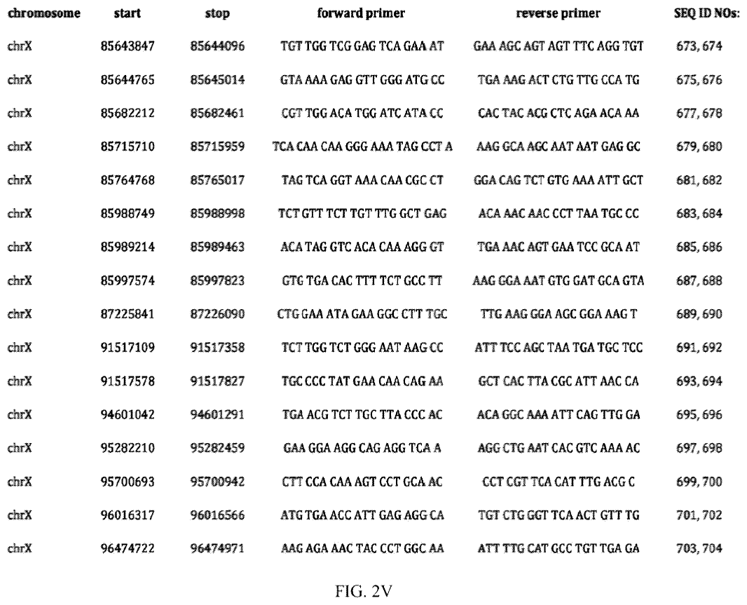

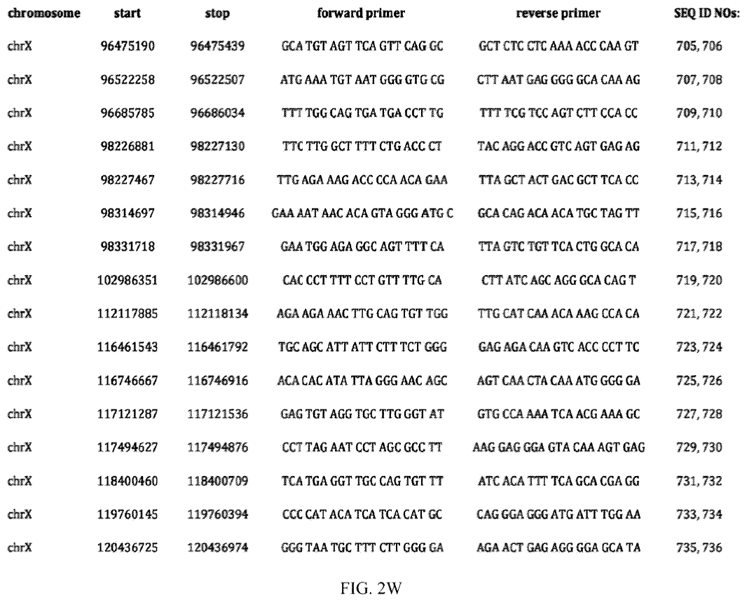

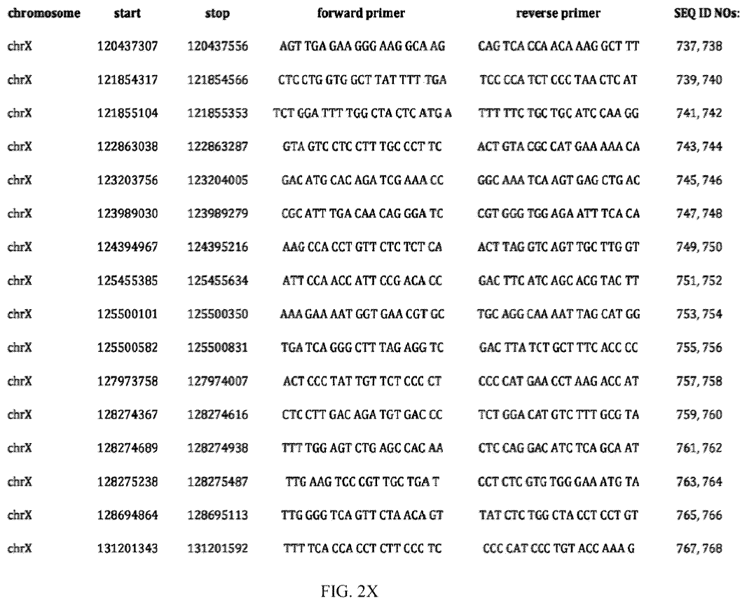

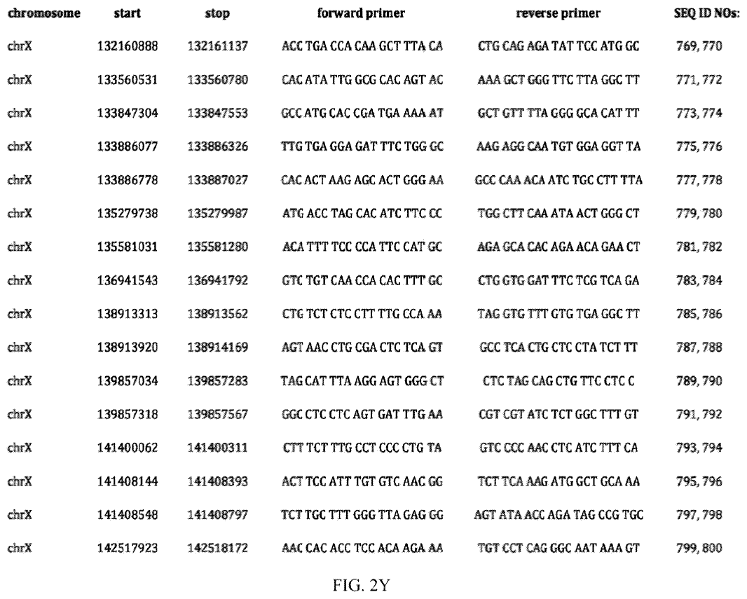

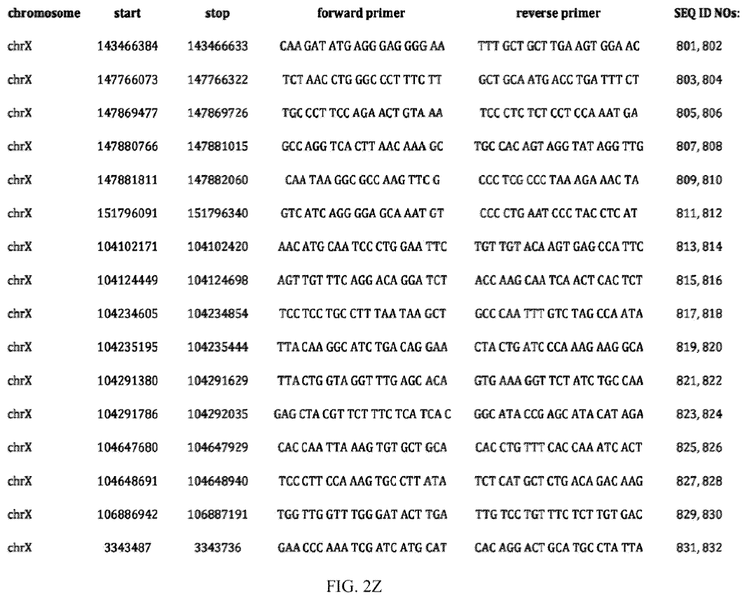

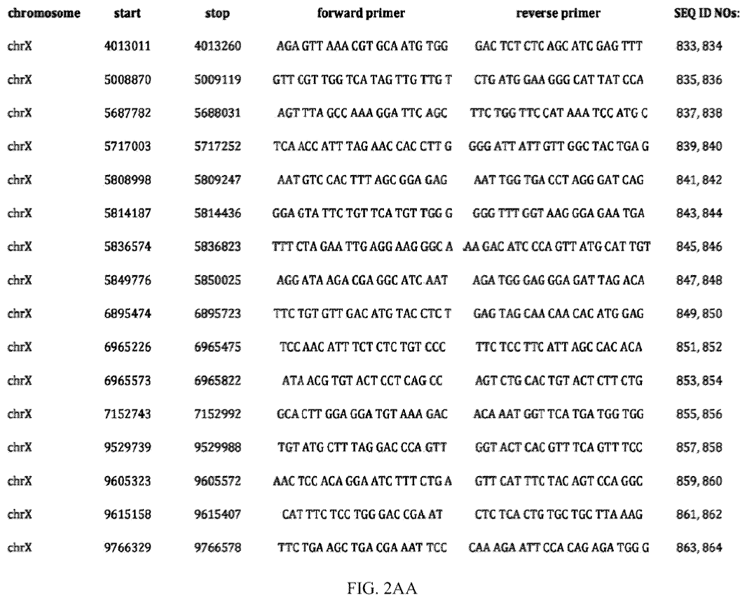

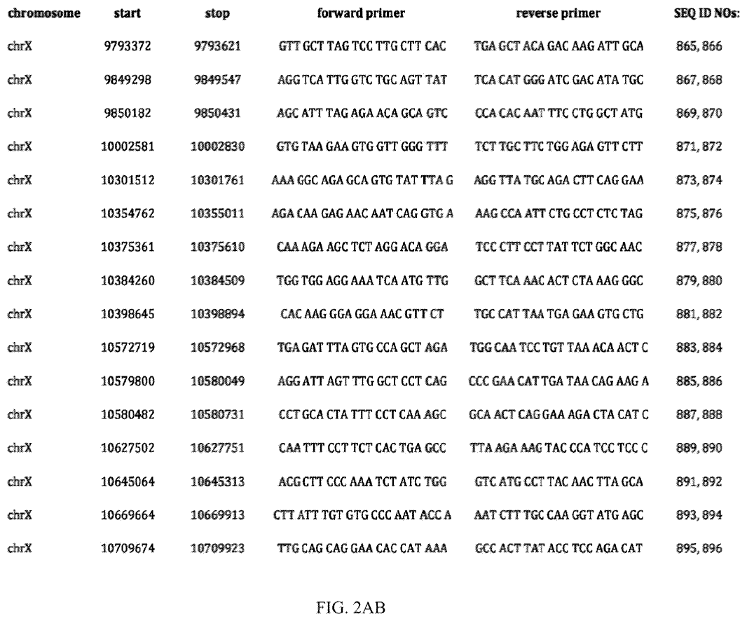

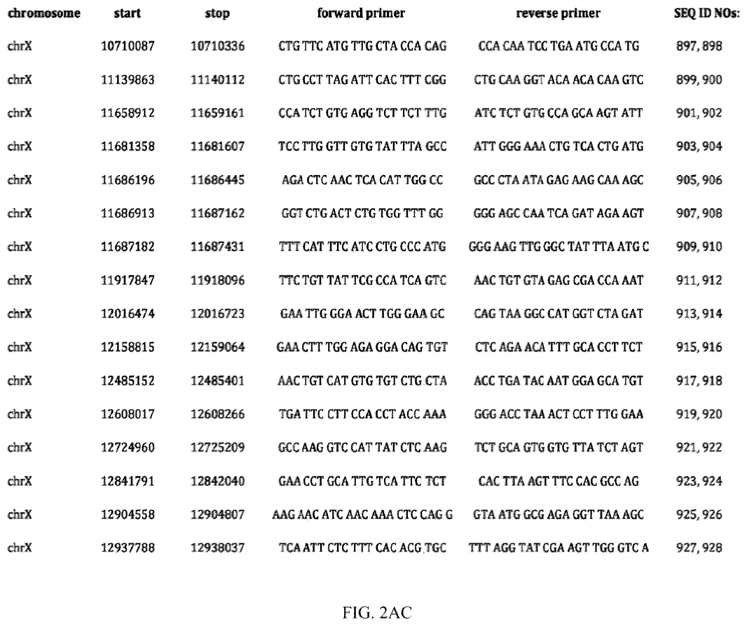

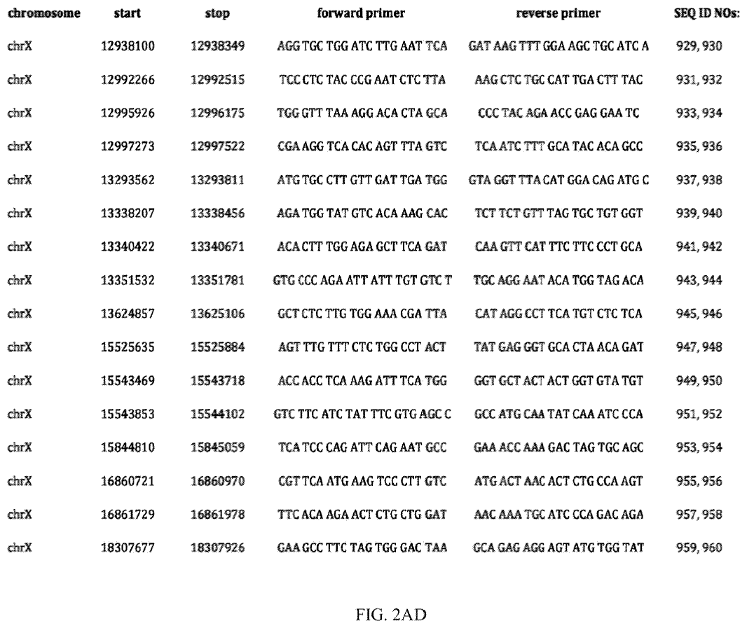

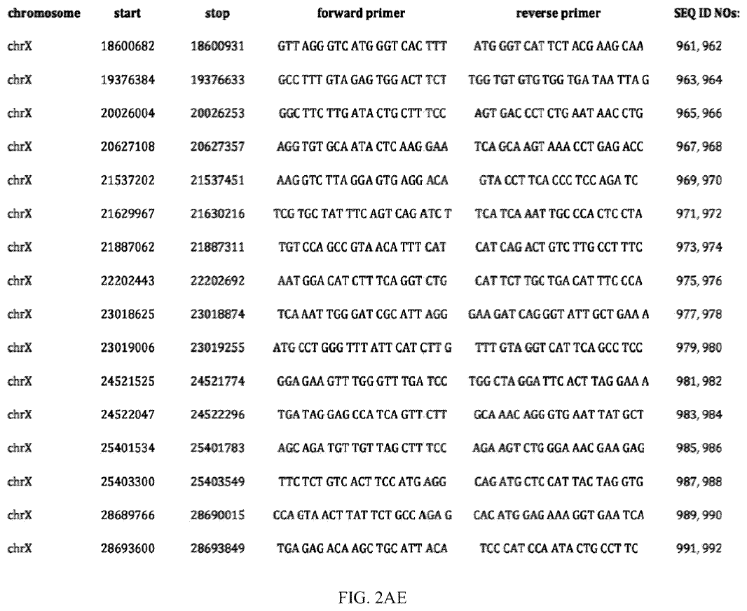

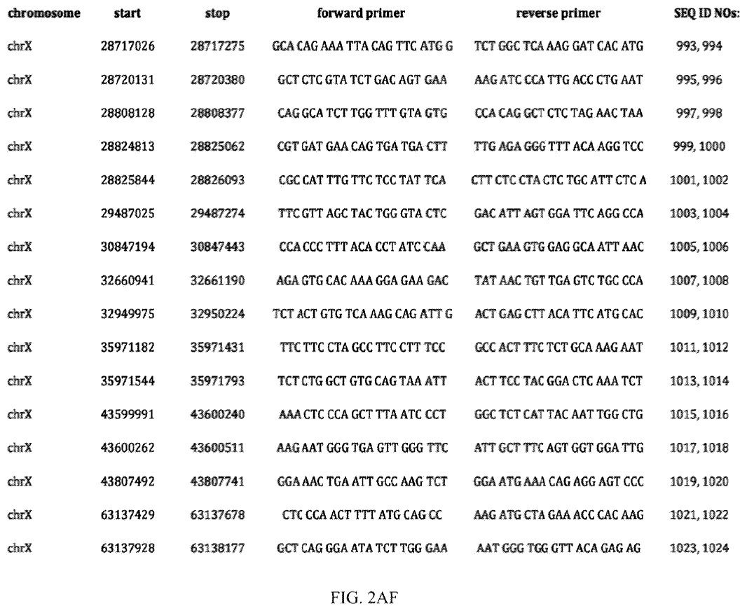

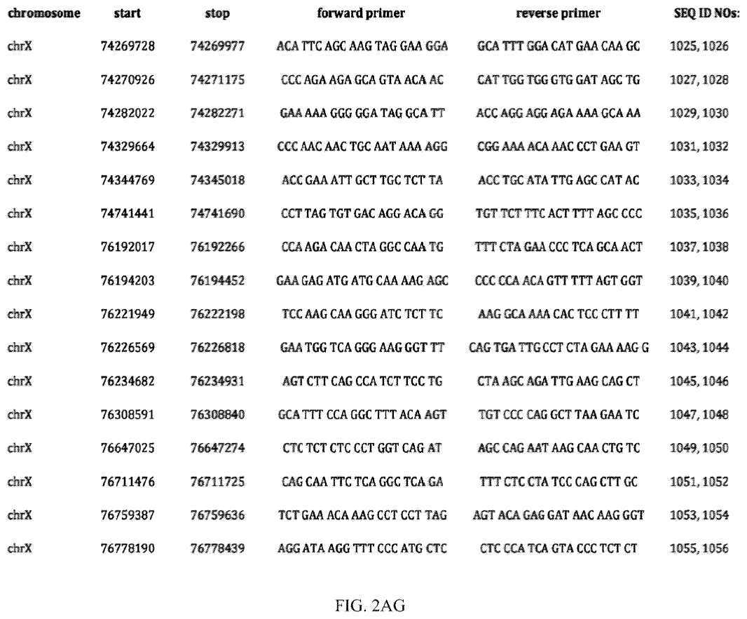

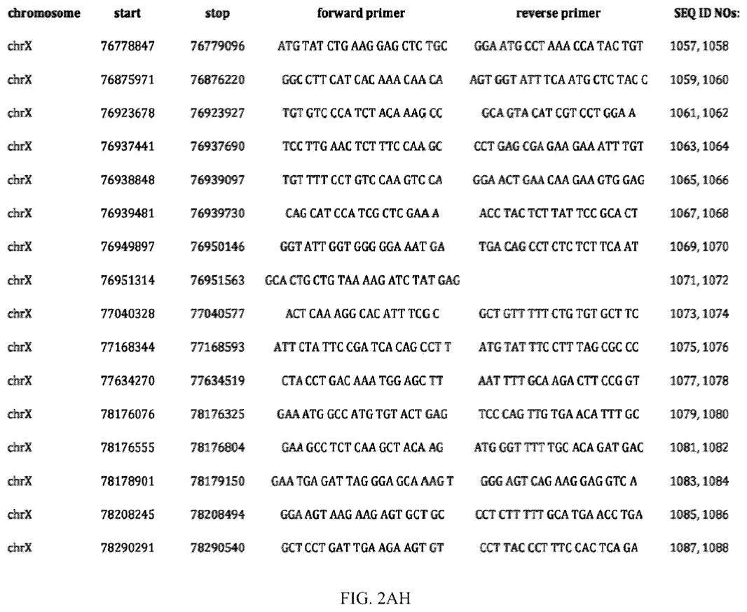

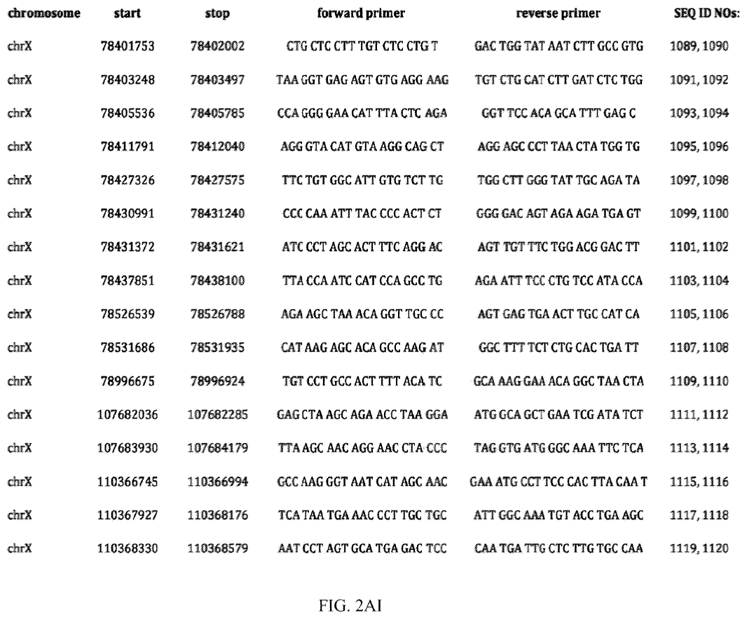

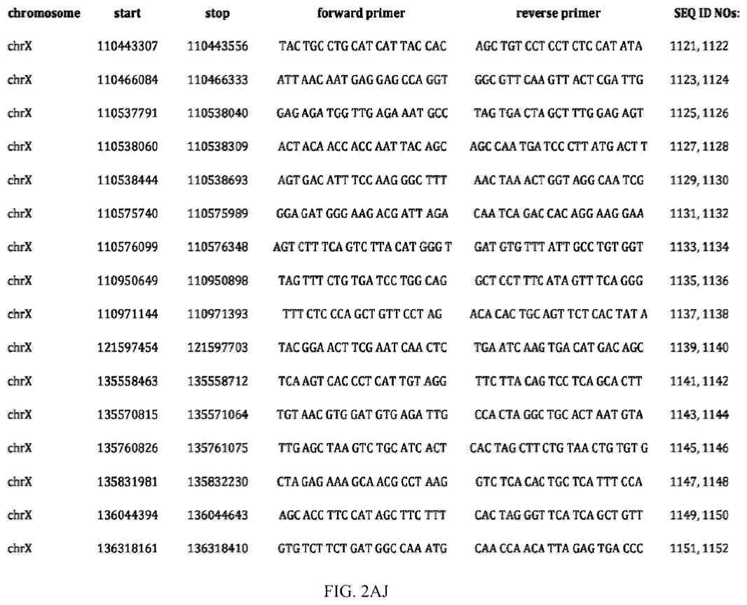

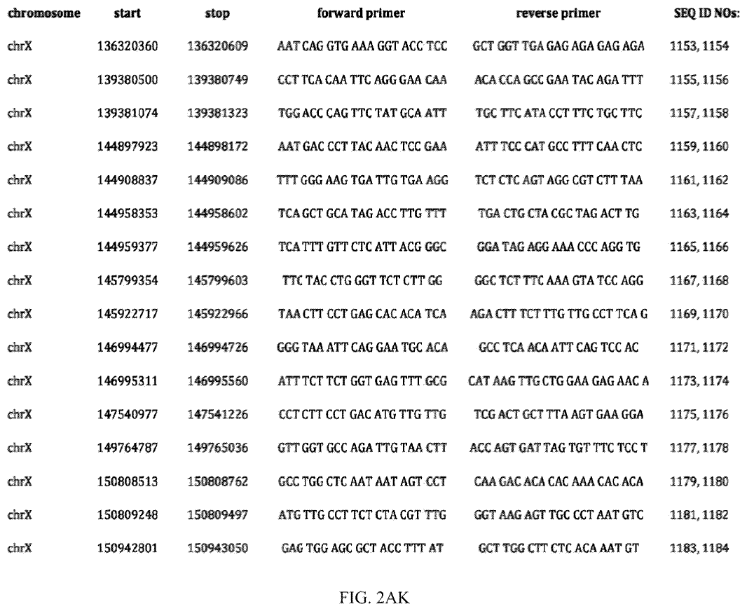

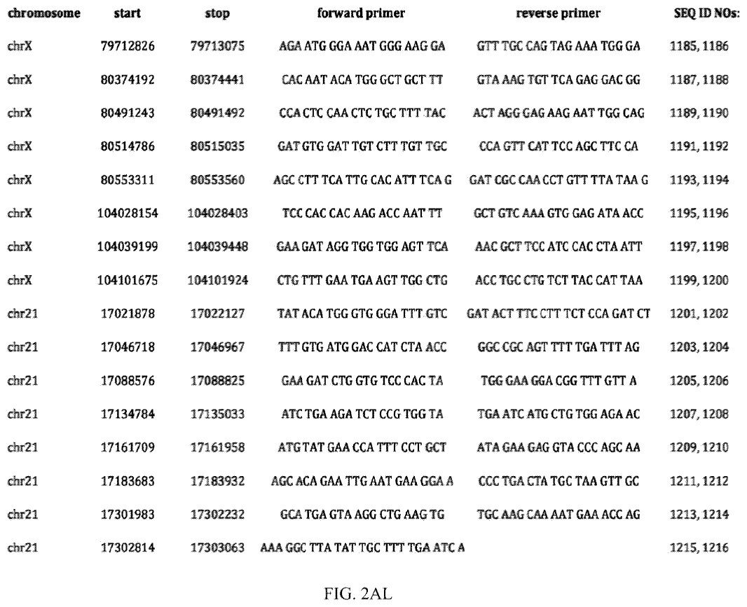

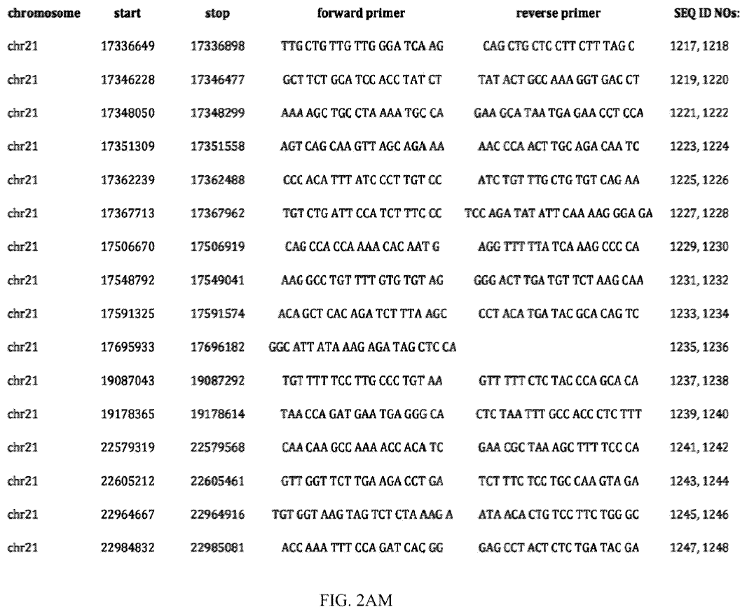

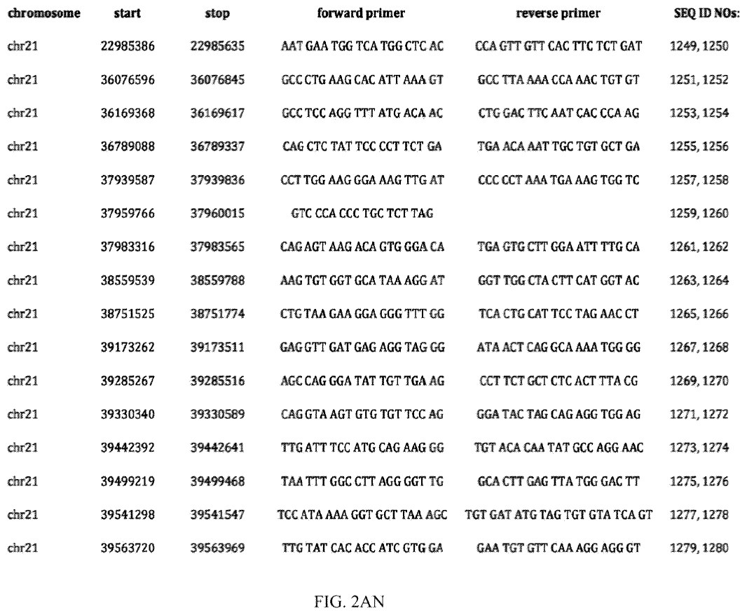

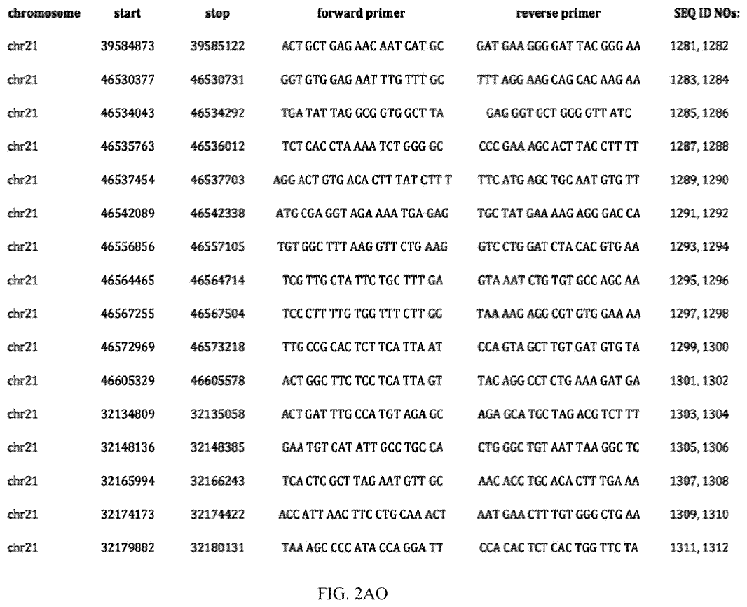

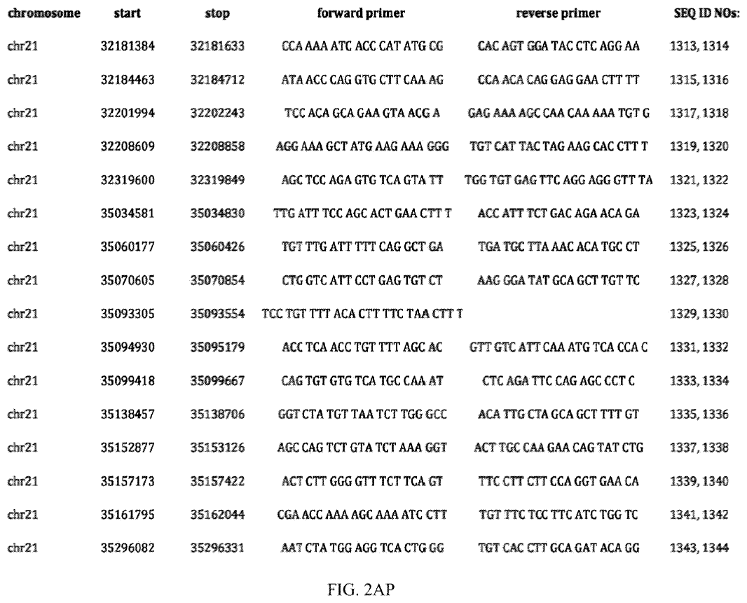

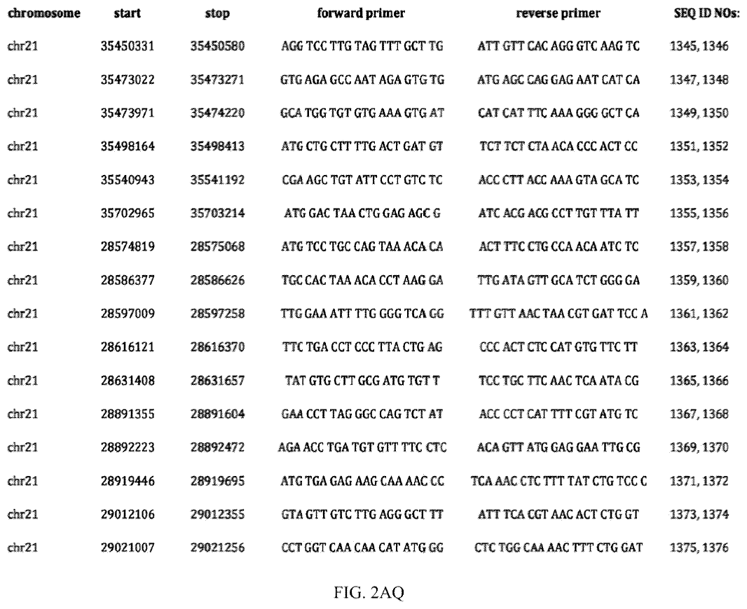

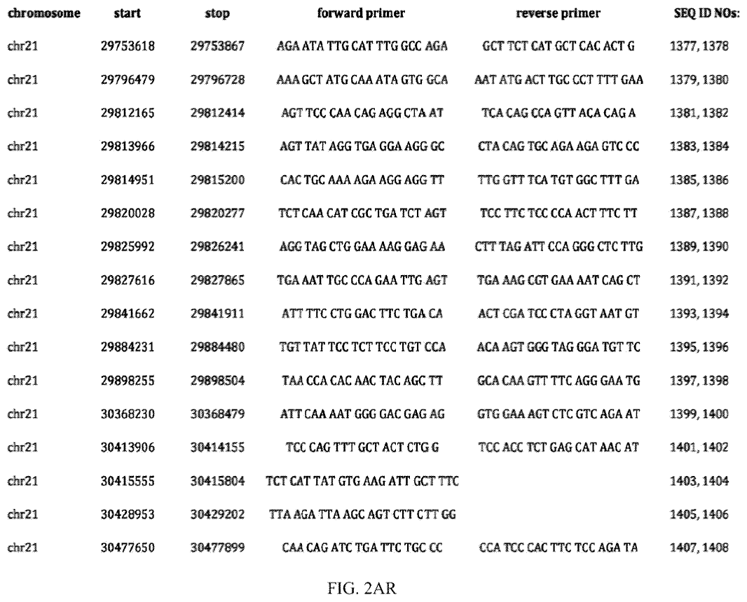

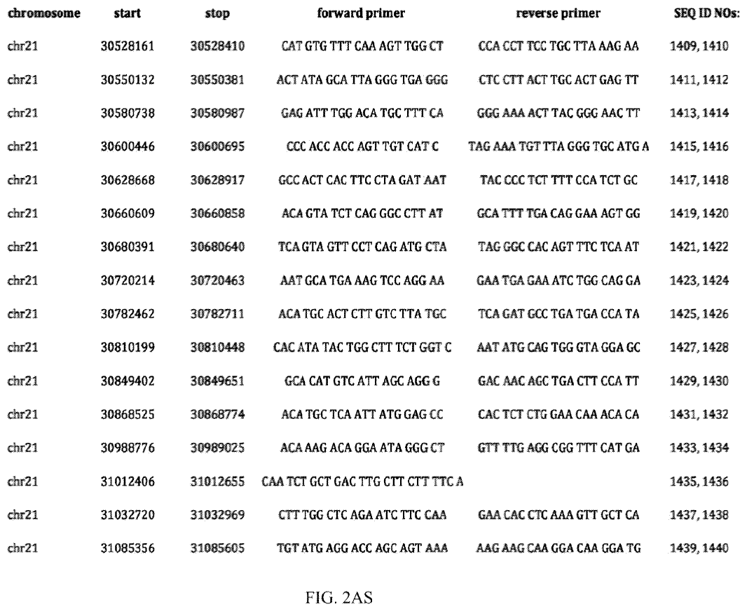

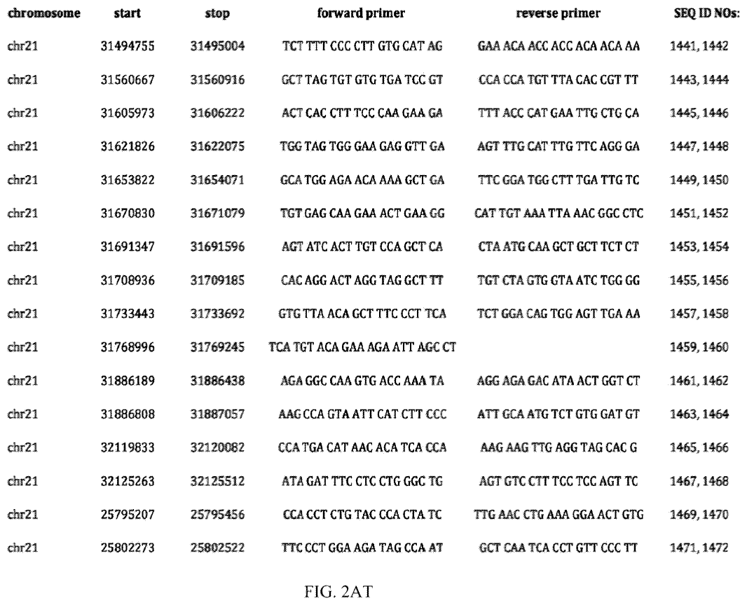

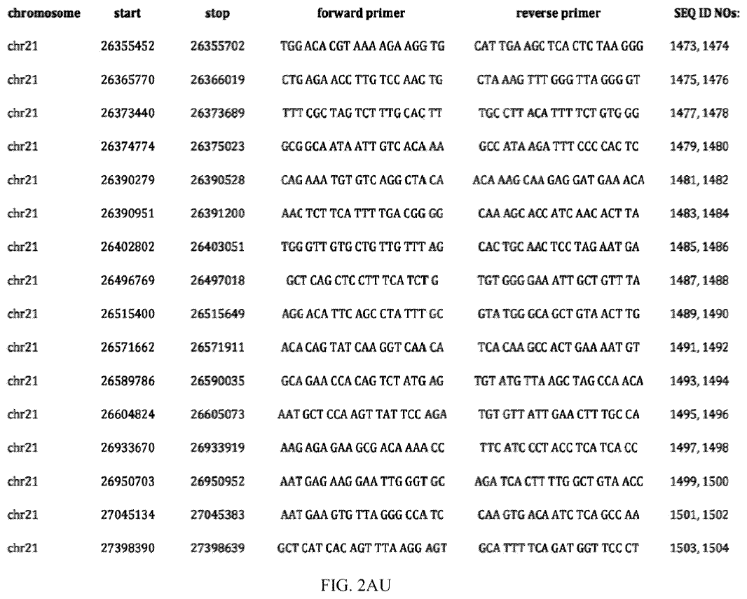

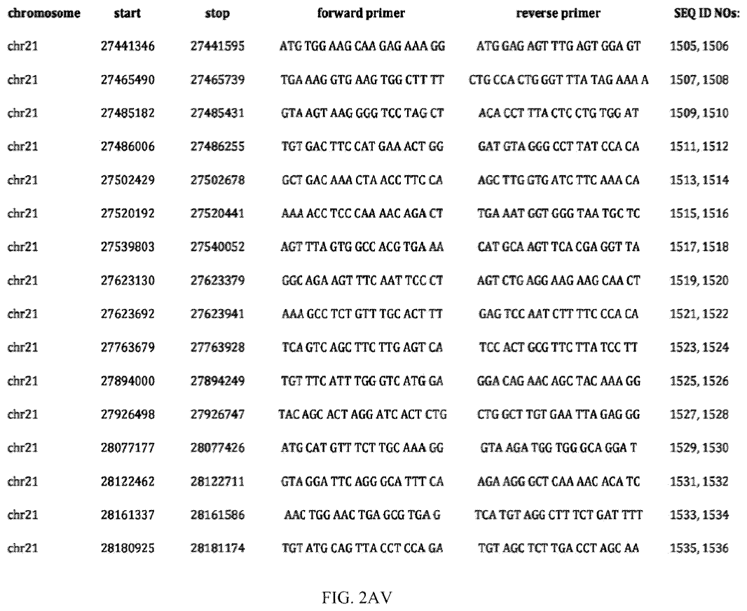

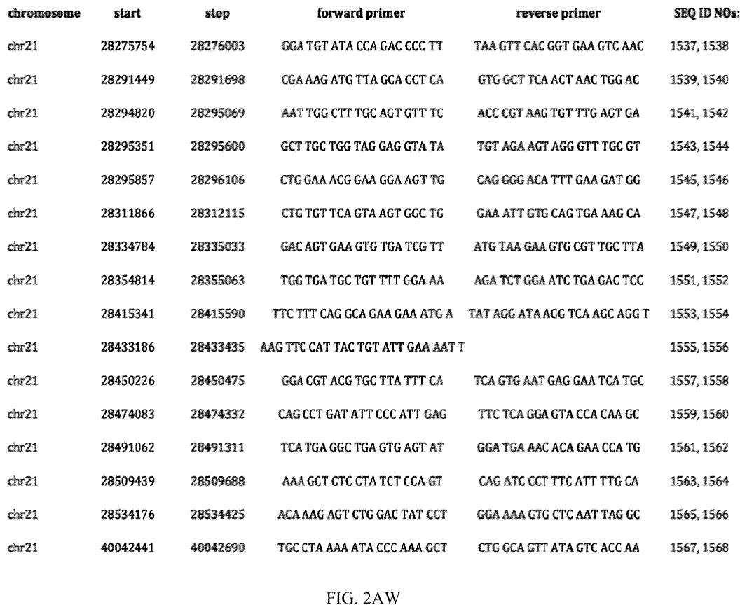

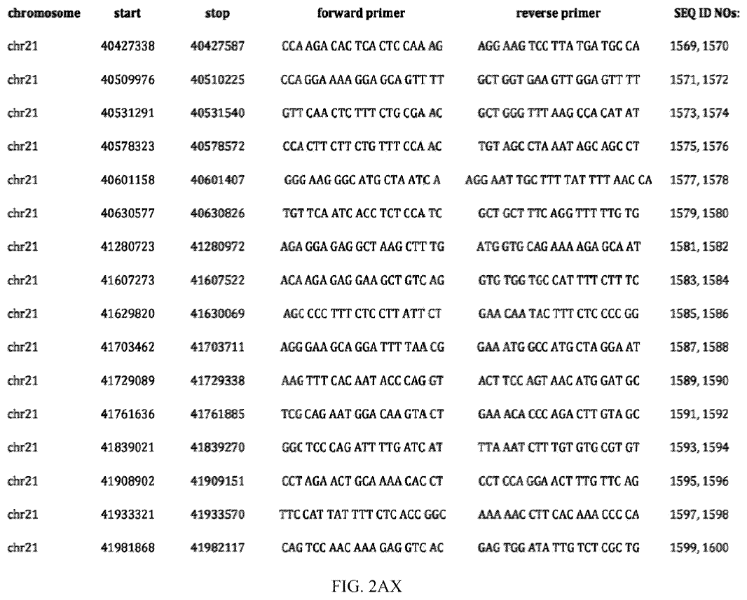

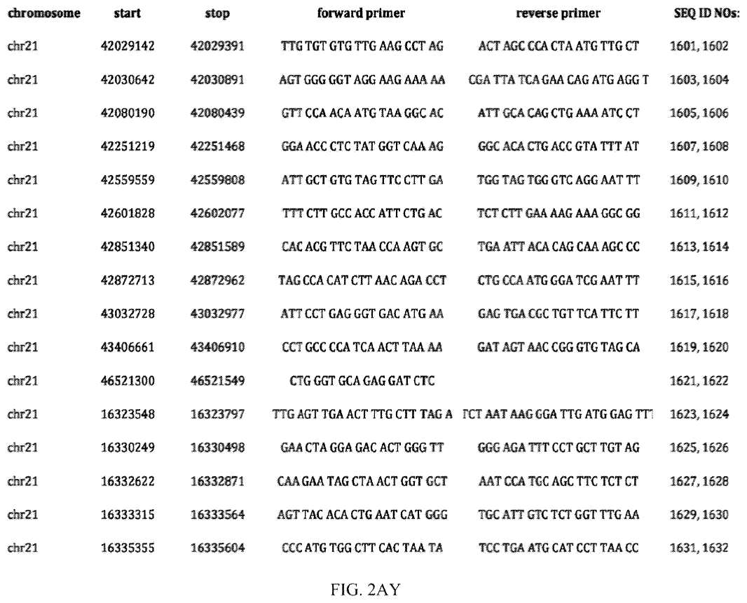

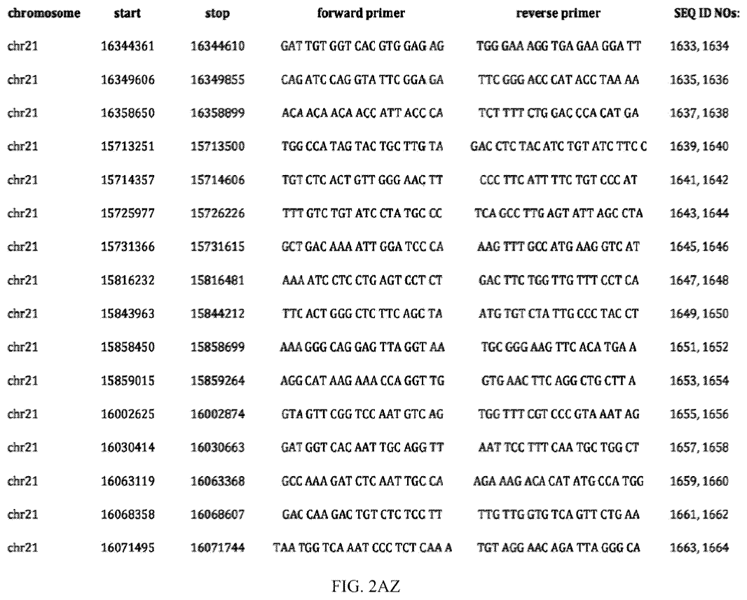

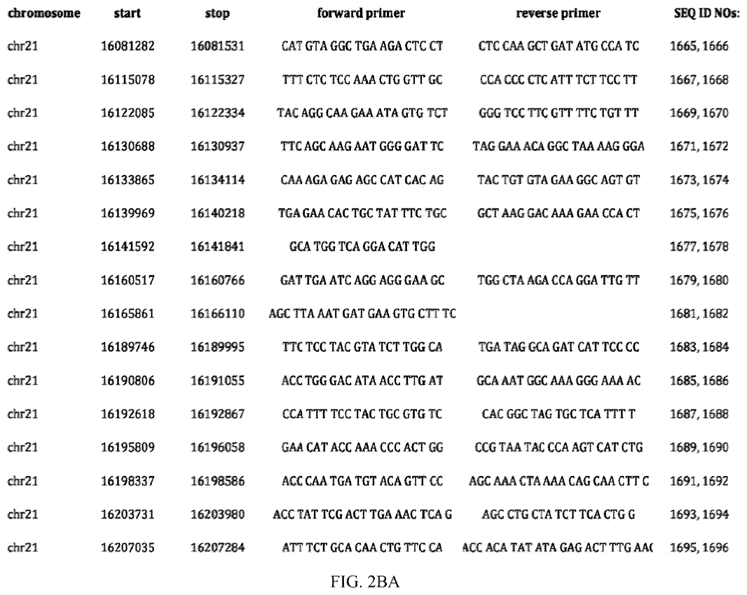

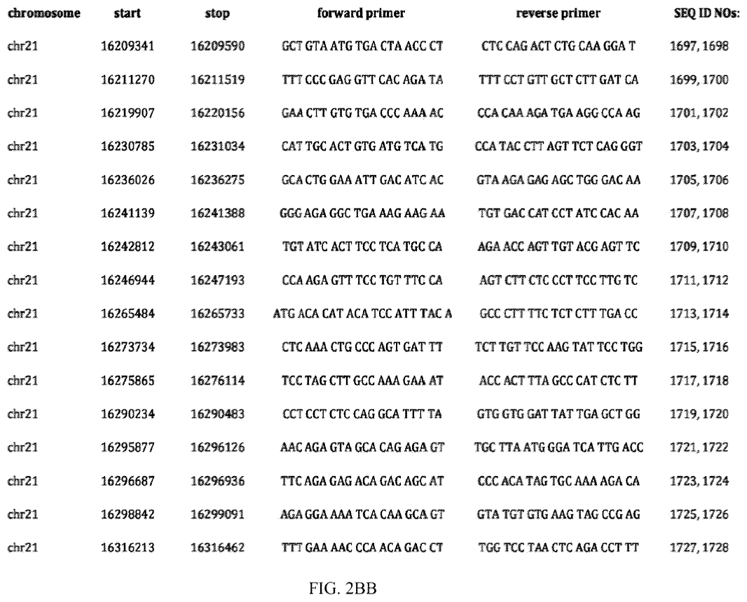

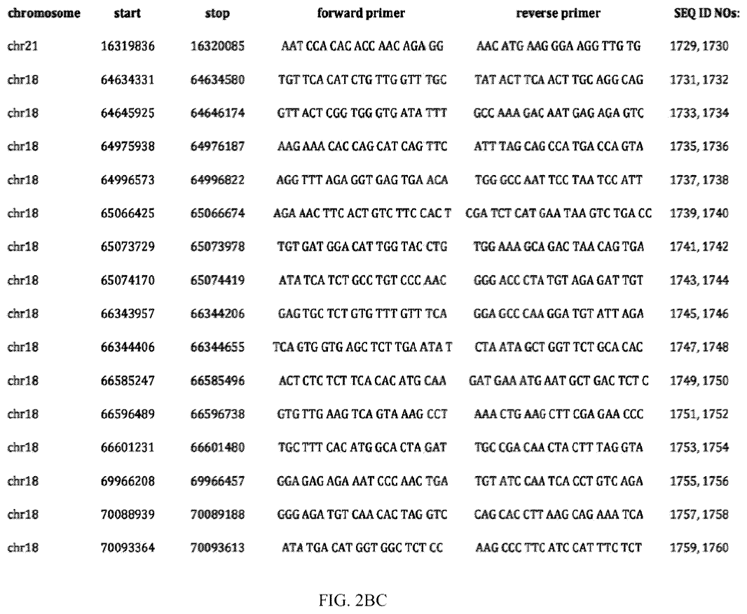

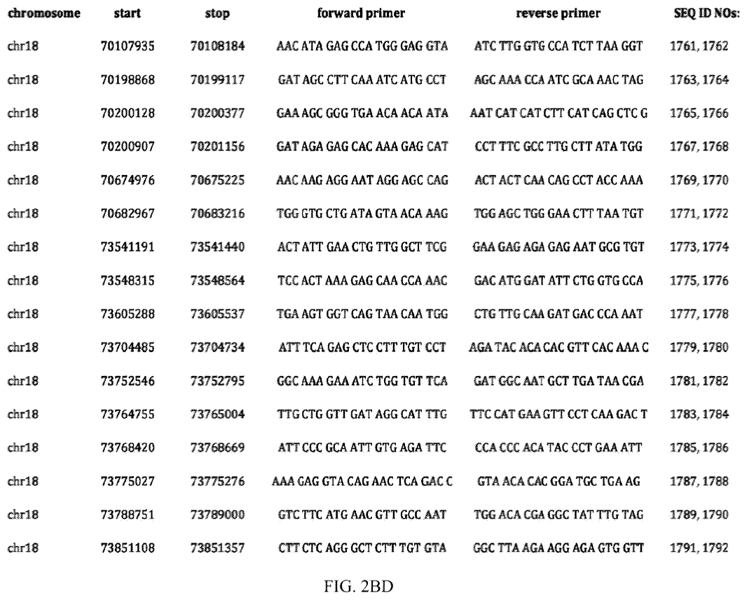

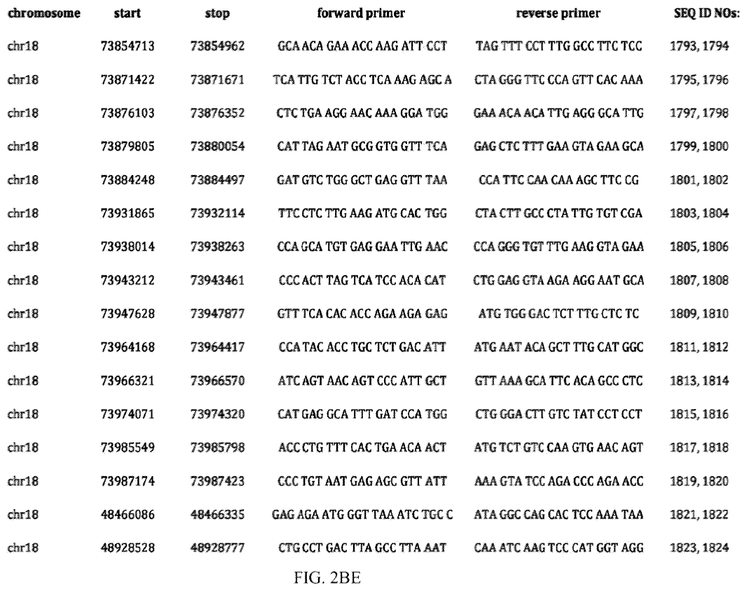

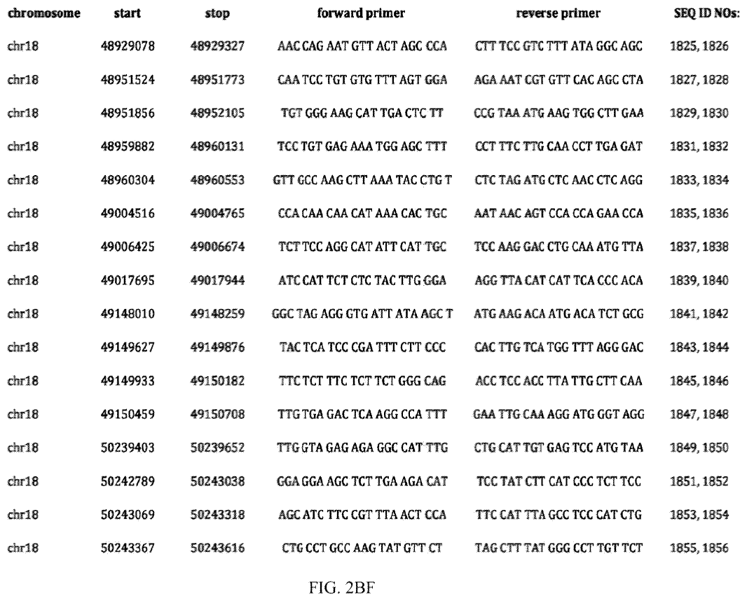

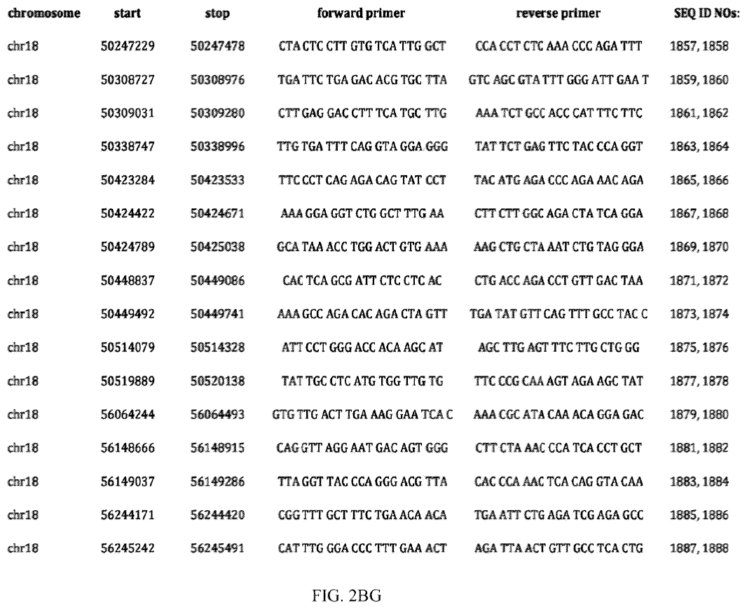

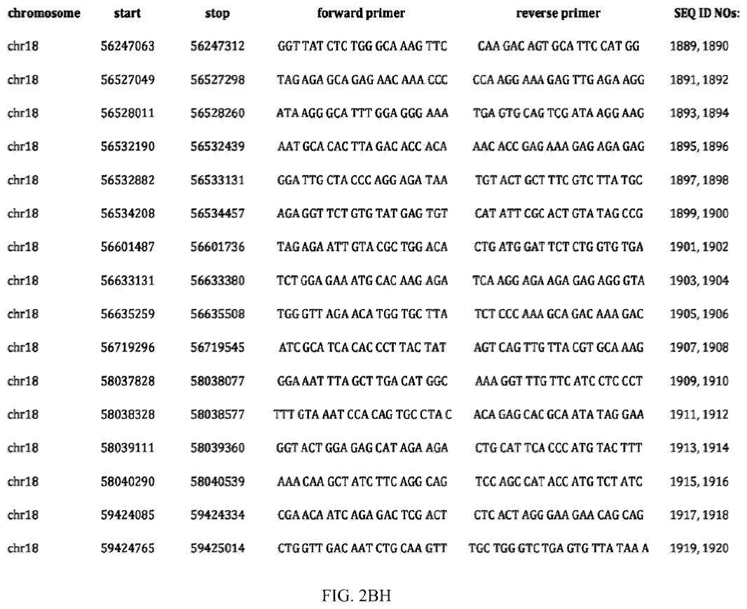

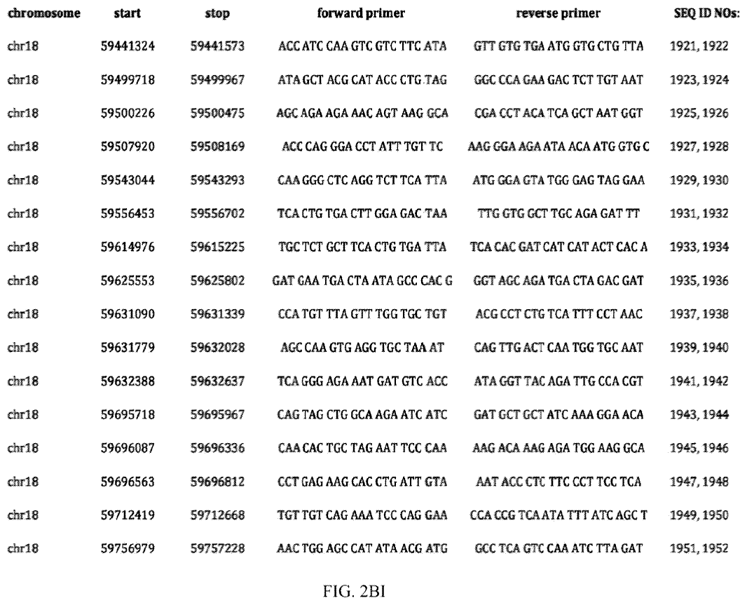

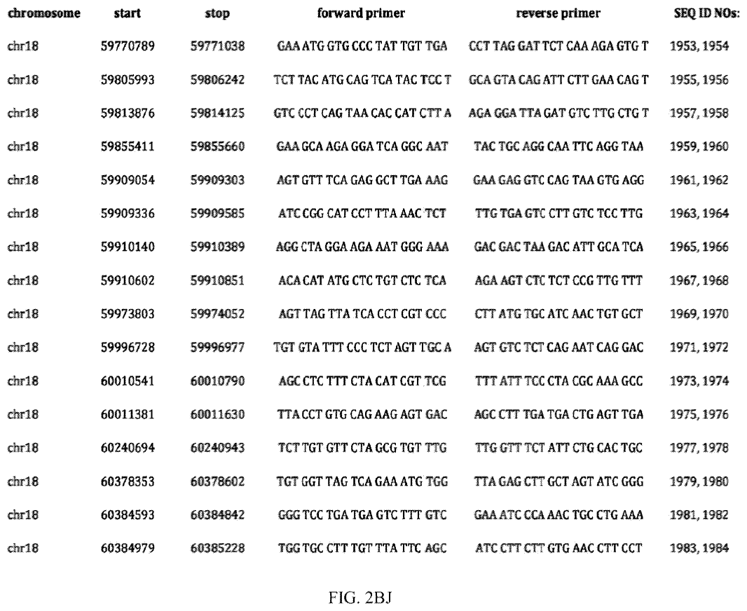

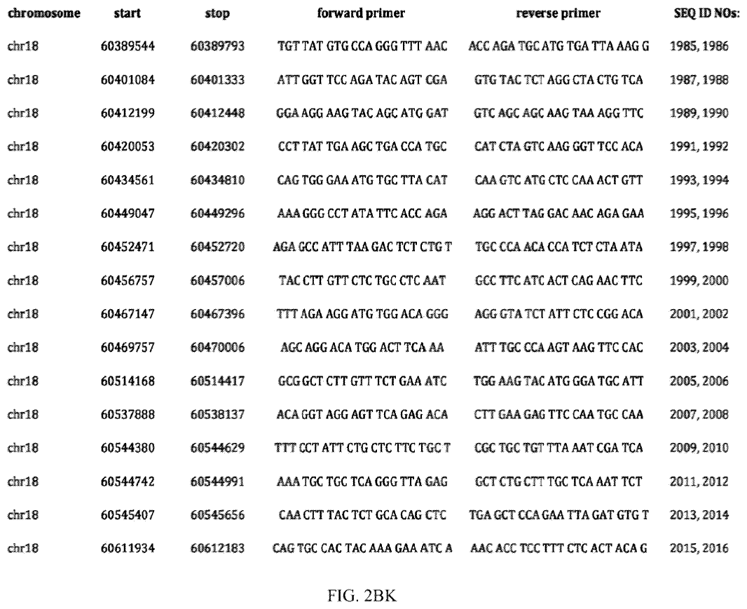

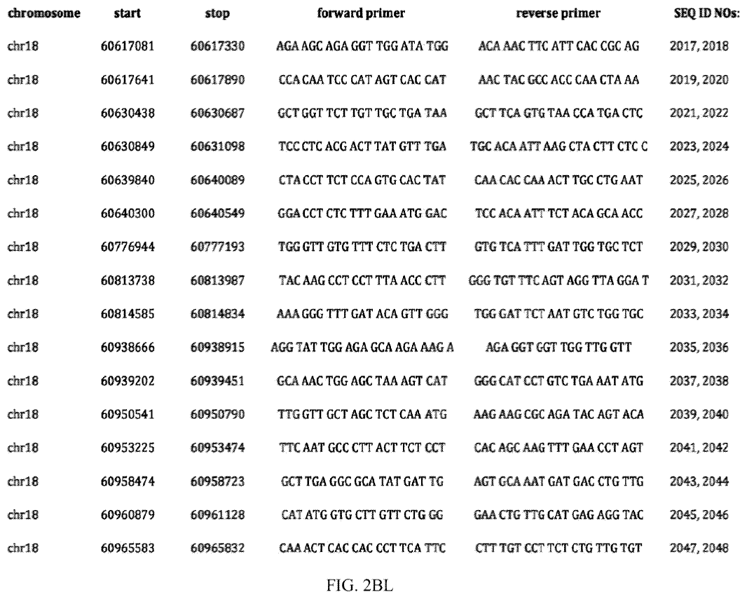

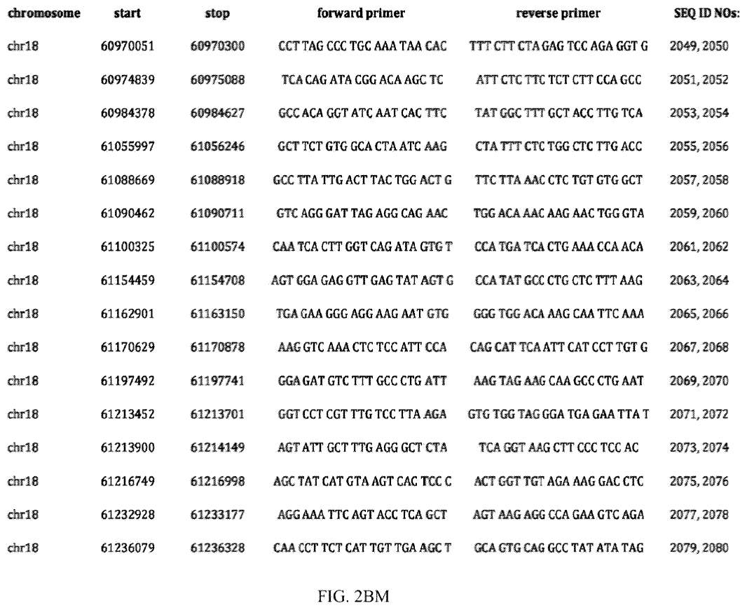

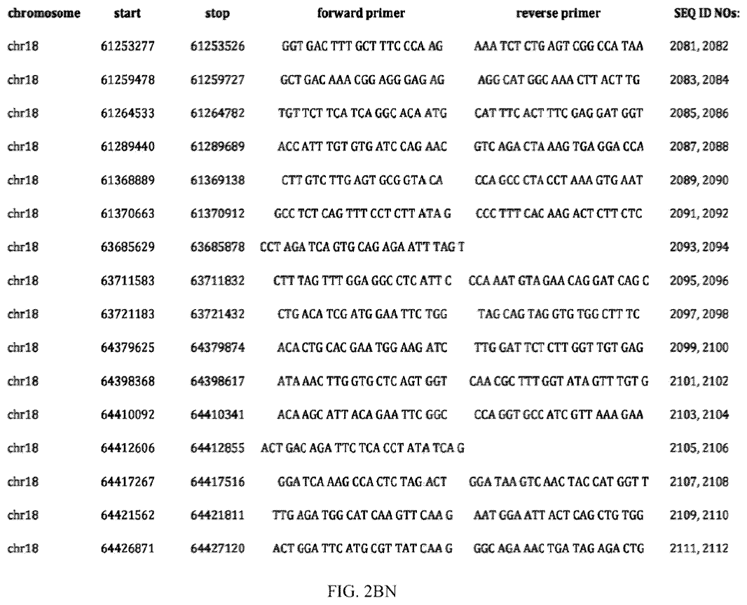

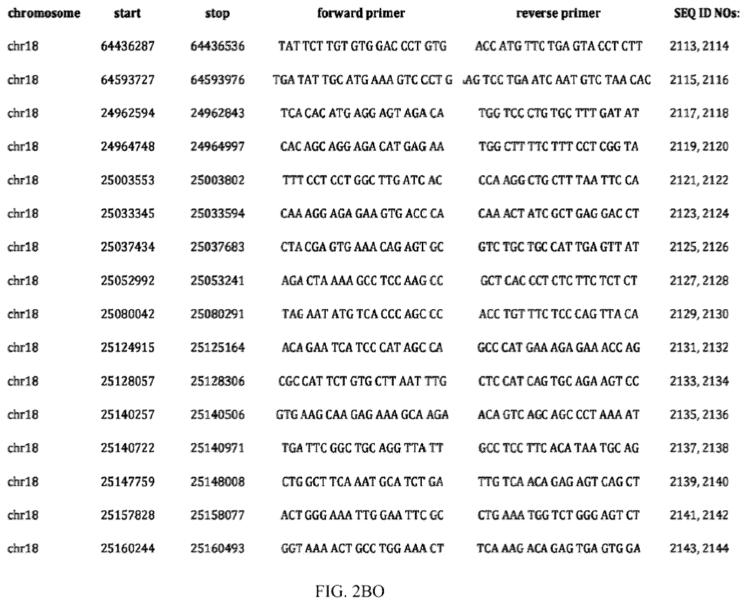

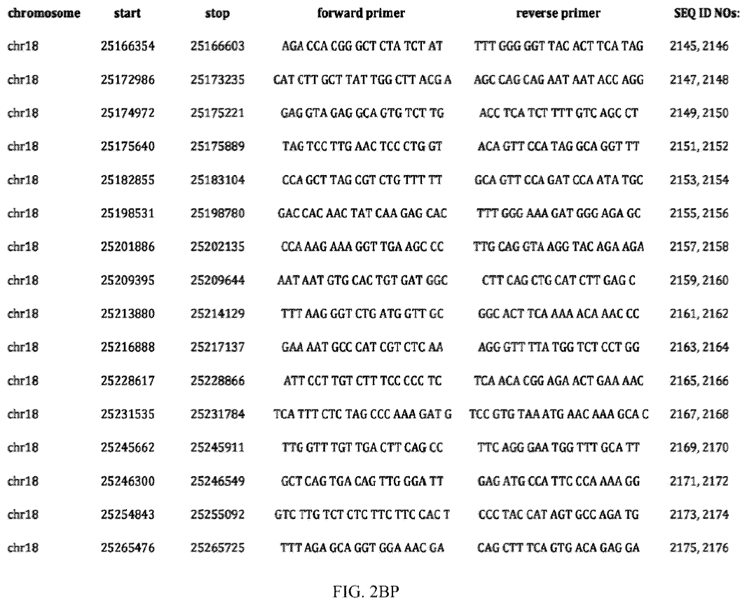

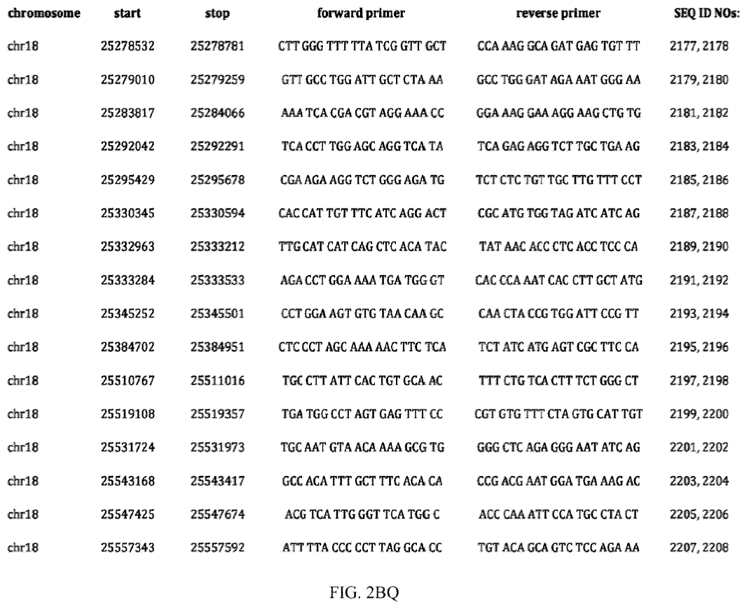

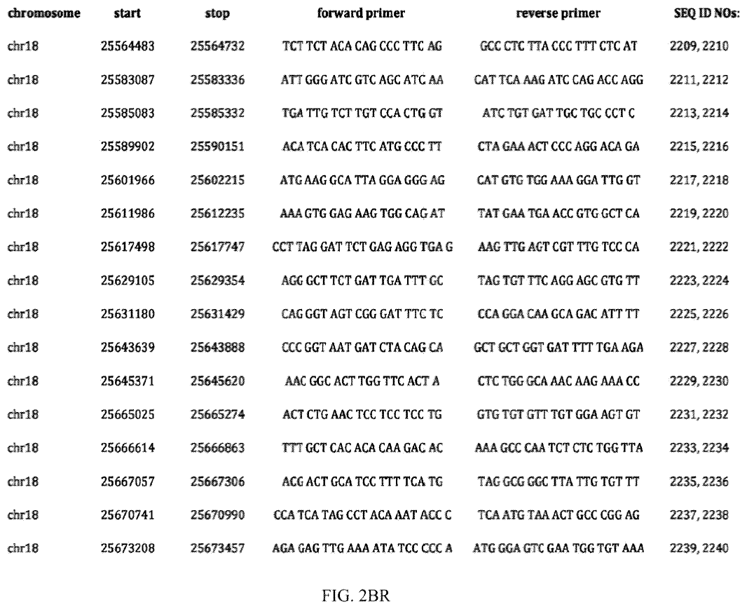

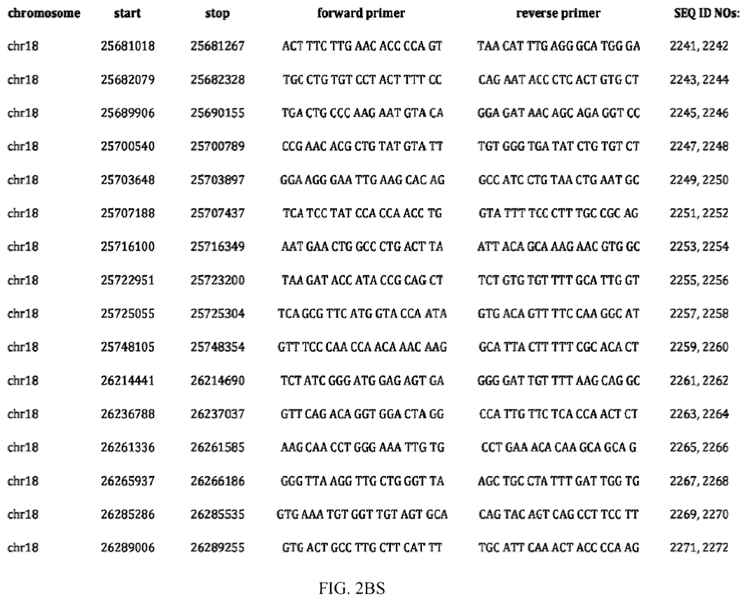

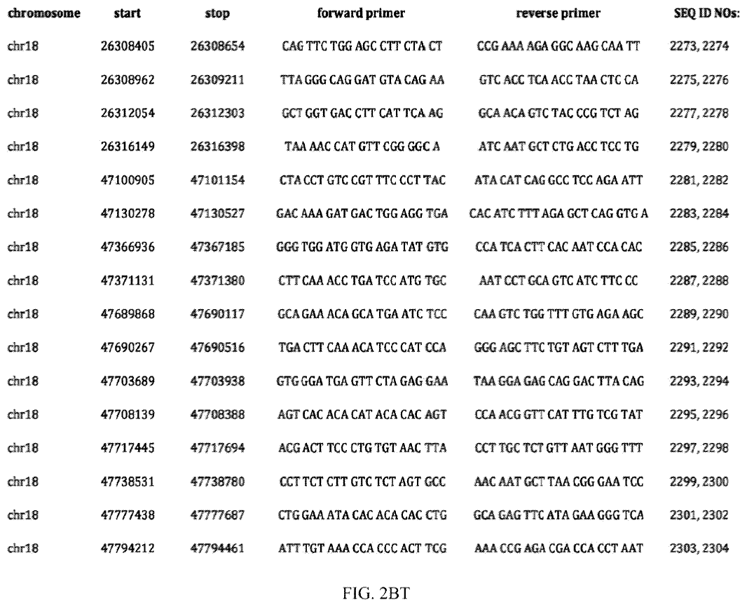

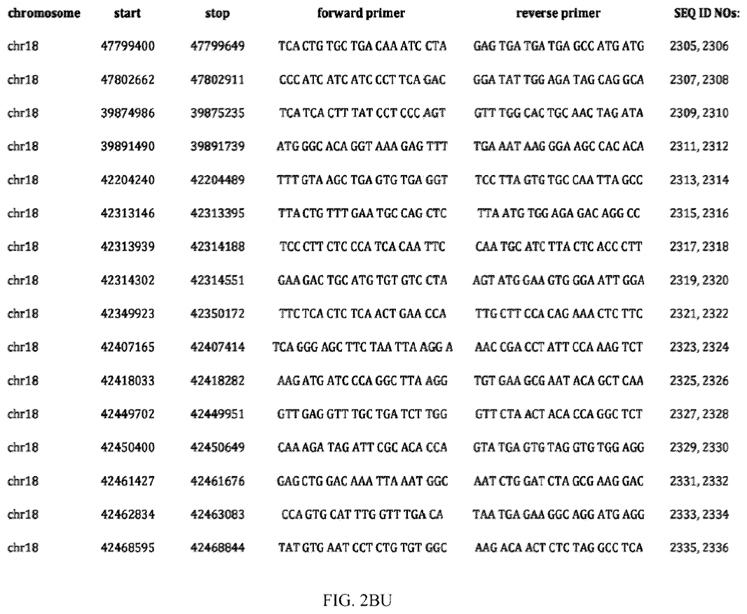

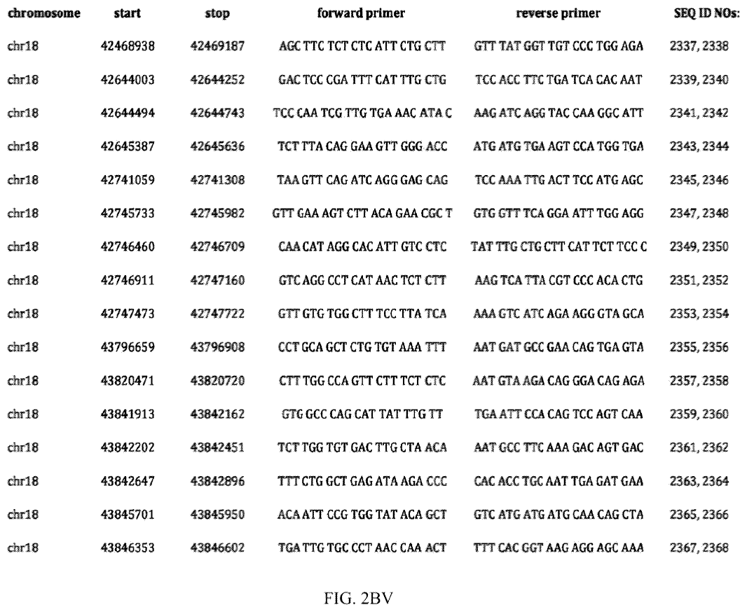

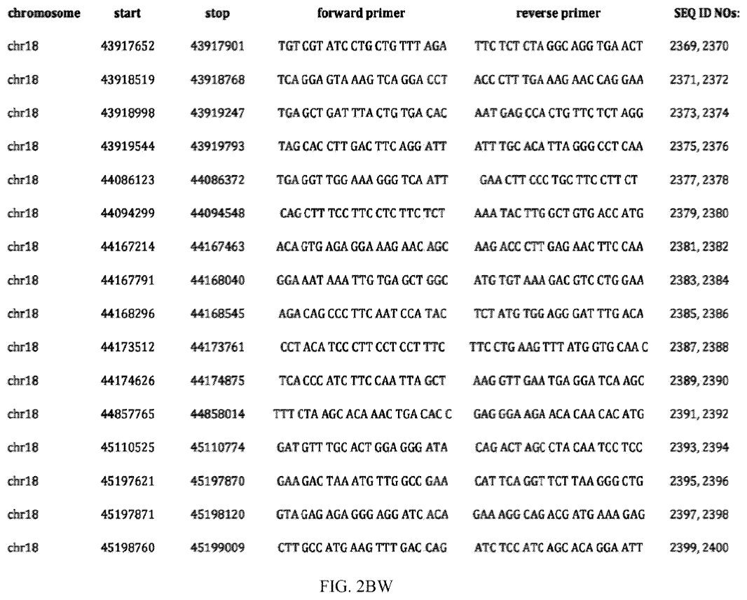

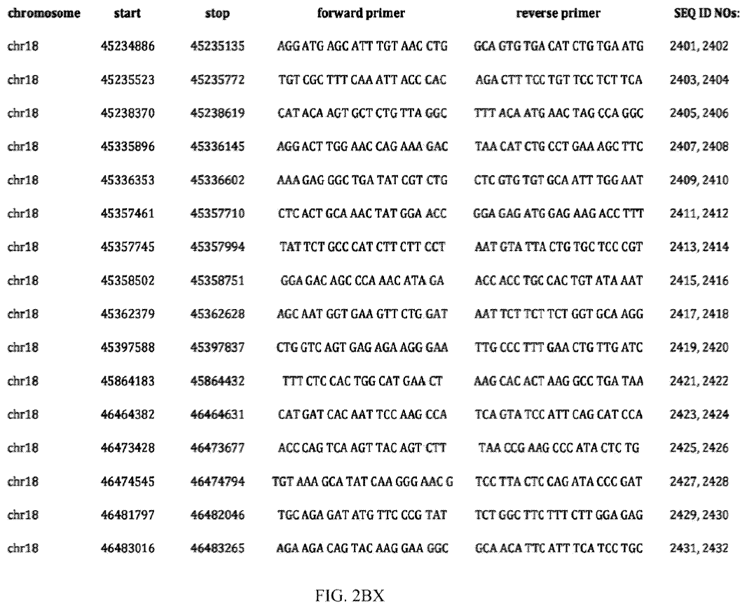

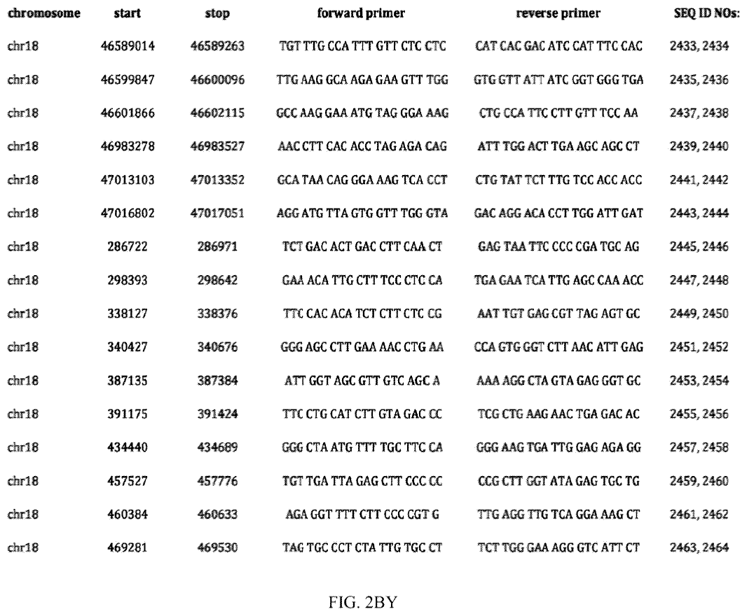

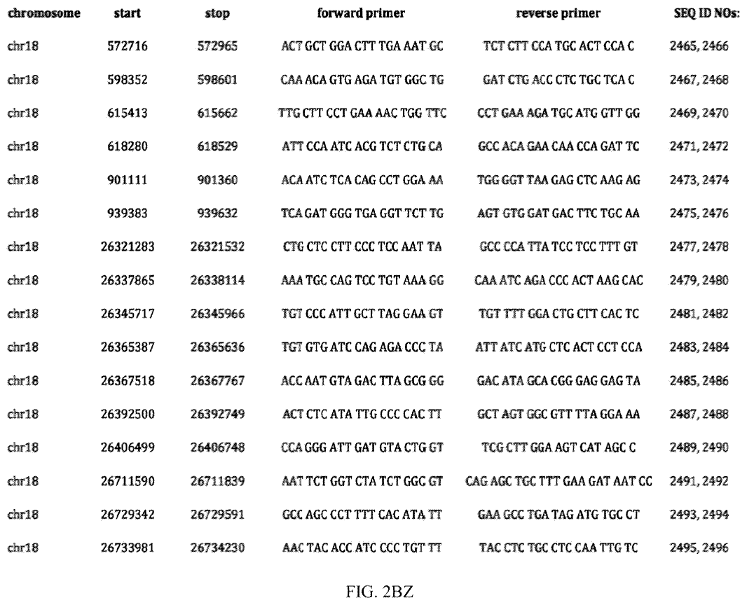

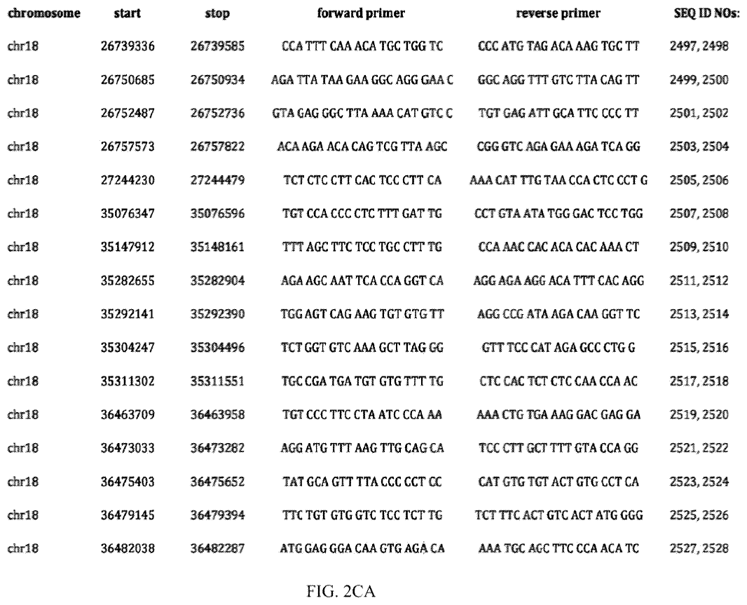

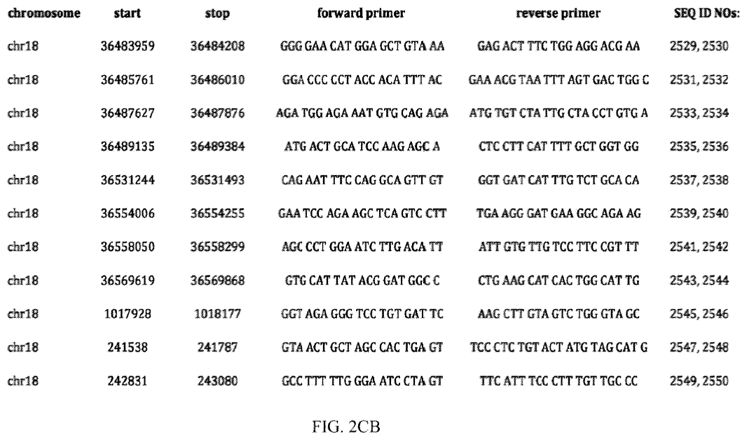

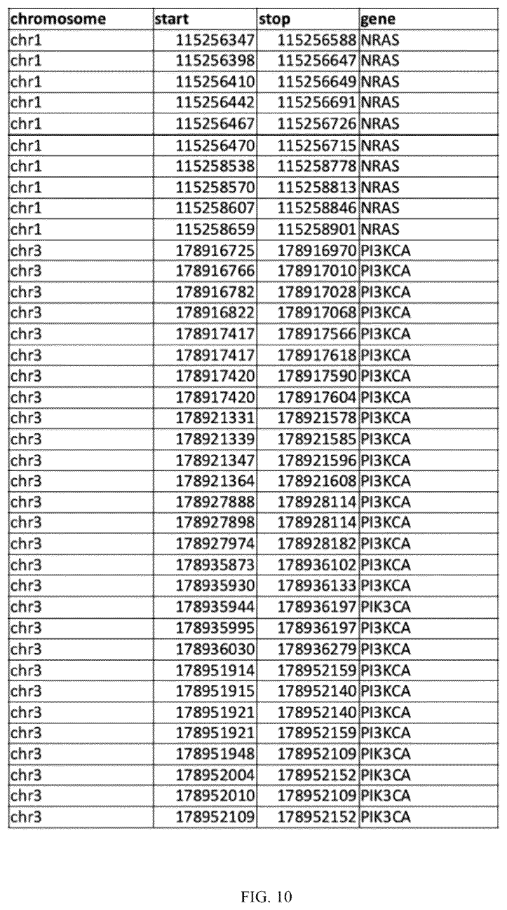

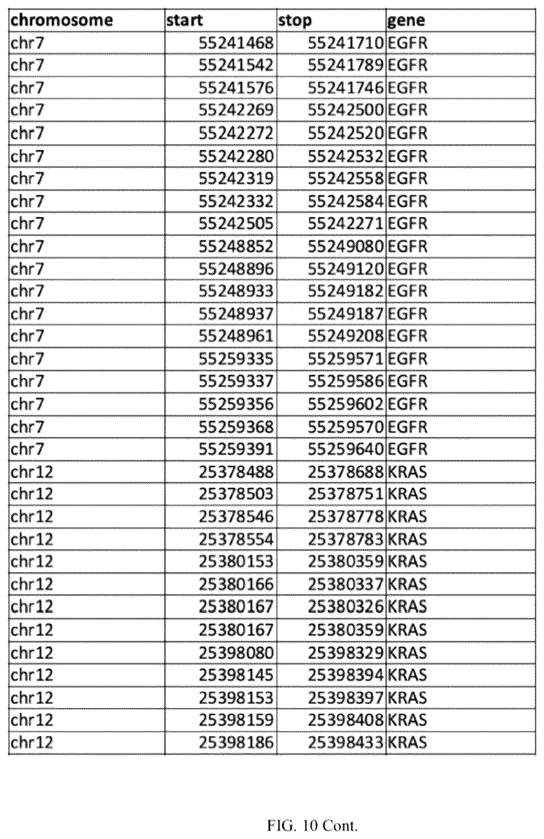

In one embodiment, the pool of TACS comprises a plurality of TACS families, wherein each member of a TACS family binds to the same tumor biomarker sequence of interest but with different start and/or stop positions on the sequence with respect to a reference coordinate system (i.e., binding of TACS family members to the target sequence is staggered) to thereby enrich for target sequences of interest, followed by massive parallel sequencing and statistical analysis of the enriched population. Typically, the reference coordinate system that is used for analyzing human genomic DNA is the human reference genome built hg19, which is publically available in the art, although other versions may be used. Alternatively, the reference coordinate system can be an artificially created genome based on built hg19 that contains only the genomic sequences of interest. Exemplary non-limiting examples of start/stop positions for TACS that bind to chromosome 13, 18, 21, X or Y are shown in FIG. 2. Exemplary non-limiting examples of start/stop positions for TACS that bind to NRAS on chromosome 1, PI3KCA on chromosome 3, EGFR on chromosome 7 or KRAS on chromosome 12 (as non-limiting examples of tumor biomarkers) are shown in FIG. 10.

Accordingly, in another aspect, the invention pertains to a method of detecting one or more tumor biomarkers in a DNA sample from a subject having or suspected of having a tumor, the method comprising:
(a) preparing a sequencing library from the DNA sample;
(b) hybridizing the sequencing library to a pool of double-stranded TArget Capture Sequences (TACS) that bind to one or more tumor biomarker sequences of interest, wherein the pool of TACS comprises a plurality of TACS families, wherein each member of a TACS family binds to the same tumor biomarker sequence of interest but with different start and/or stop positions on the sequence with respect to a reference coordinate system, and further wherein:
   (i) each member sequence within the pool of TACS is between 100-500 base pairs in length, each member sequence having a 5′ end and a 3′ end;
   (ii) preferably each member sequence binds to the tumor biomarker sequence of interest at least 50 base pairs away, on both the 5′ end and the 3′ end, from regions harboring Copy Number Variations (CNVs), Segmental duplications or repetitive DNA elements; and
   (iii) the GC content of the pool of TACS is between 19% and 80%, as determined by calculating the GC content of each member within the pool of TACS;
(c) isolating members of the sequencing library that bind to the pool of TACS to obtain an enriched library;
(d) amplifying and sequencing the enriched library; and
(e) performing statistical analysis on the enriched library sequences, optionally utilizing only fragments of a specific size range, to thereby detect the tumor biomarker(s) in the DNA sample.

The TACS-enrichment based method of the disclosure can be used in the detection of a wide variety of genetic abnormalities. In one embodiment, the genetic abnormality is a chromosomal aneuploidy (such as a trisomy, a partial trisomy or a monosomy). In other embodiments, the genomic abnormality is a structural abnormality, including but not limited to copy number changes including microdeletions and microduplications, insertions, translocations, inversions and small-size mutations including point mutations and mutational signatures. In another embodiment, the genetic abnormality is a chromosomal mosaicism.

Further aspects and features of the methods of the disclosure are described in the subsections below.

### TArget Capture Sequence Design

As used herein, the term “TArget Capture Sequences” or “TACS” refers to short DNA sequences that are complementary to the region(s) of interest on a genomic sequence(s) of interest (e.g., chromosome(s) of interest) and which are used as “bait” to capture and enrich the region of interest from a large library of sequences, such as a whole genomic sequencing library prepared from a biological sample. In addition to the features of the families of TACS described above (e.g., staggered binding to the genomic sequence of interest), a pool of TACS is used for enrichment wherein the sequences within the pool have been optimized with regard to: (i) the length of the sequences; (ii) the distribution of the TACS across the region(s) of interest; and (iii) the GC content of the TACS. The number of sequences within the TACS pool (pool size) has also been optimized.

It has been discovered that TACS having a length of 100-500 base pairs are optimal to maximize enrichment efficiency. In various other embodiments, each sequence within the pool of TACS is between 150-260 base pairs, 100-200 base pairs, 200-260 base pairs, 100-350 bp in length, or 100-500 bp in length. In preferred embodiments, the length of the TACS within the pool is at least 250 base pairs, or is 250 base pairs or is 260 base pairs or is 280 base pairs. It will be appreciated by the ordinarily skilled artisan that a slight variation in TACS size typically can be used without altering the results (e.g., the addition or deletion of a few base pairs on either end of the TACS); accordingly, the base pair lengths given herein are to be considered “about” or “approximate”, allowing for some slight variation (e.g., 1-5%) in length. Thus, for example, a length of “250 base pairs” is intended to refer to “about 250 base pairs” or “approximately 250 base pairs”, such that, for example, 248 or 252 base pairs is also encompassed.

The distribution of the TACS across each region or chromosome of interest has been optimized to avoid, if applicable, high copy repeats, low copy repeats and copy number variants, while at the same time also being able to target informative single nucleotide polymorphisms (SNPs) in order to enable both aneuploidy, or structural copy number change detection, and fraction of interest estimation. Accordingly, each sequence within the TACS pool is designed such that the 5′ end and the 3′ end are each at least 50 base pairs away from regions in the genome that are known to harbor one or more of the following genomic elements: Copy Number Variations (CNVs), Segmental duplications and/or repetitive DNA elements (such as transposable elements or tandem repeat areas). In various other embodiments, each sequence within the TACS pool is designed such that the 5′ end and the 3′ end are each at least 50, 100, 150, 200, 250, 300, 400 or 500 base pairs away from regions in the genome that are known to harbor one or more of the aforementioned elements.

The term “Copy Number Variations” is a term of art that refers to a form of structural variation in the human genome in which there can be alterations in the DNA of the genome in different individuals that can result in a fewer or greater than normal number of a section(s) of the genome in certain individuals. CNVs correspond to relatively large regions of the genome that may be deleted (e.g., a section that normally is A-B-C-D can be A-B-D) or may be duplicated (e.g., a section that normally is A-B-C-D can be A-B-C-C-D). CNVs account for roughly 13% of the human genome, with each variation ranging in size from about 1 kilobase to several megabases in size.

The term “Segmental duplications” (also known as “low-copy repeats”) is also a term of art that refers to blocks of DNA that range from about 1 to 400 kilobases in length that occur at more than one site within the genome and typically share a high level (greater than 90%) of sequence identity. Segmental duplications are reviewed in, for example, Eichler. E. E. (2001) Trends Genet. 17:661-669.

The term “repetitive DNA elements” (also known as “repeat DNA” or “repeated DNA”) is also a term of art that refers to patterns of DNA that occur in multiple copies throughout the genome. The term “repetitive DNA element” encompasses terminal repeats, tandem repeats and interspersed repeats, including transposable elements. Repetitive DNA elements in NGS is discussed further in, for example, Todd, J. et al. (2012) Nature Reviews Genet. 13:36-46.

The TACS are designed with specific GC content characteristics in order to minimize data GC bias and to allow a custom and innovative data analysis pipeline. It has been determined that TACS with a GC content of 19-80% achieve optimal enrichment and perform best with cell free DNA. Within the pool of TACS, different sequences can have different % GC content, although to be selected for inclusion with the pool, the % GC content of each sequence is chosen as between 19-80%, as determined by calculating the GC content of each member within each family of TACS. That is, every member within each family of TACS has a % GC content within the given percentage range (e.g., between 19-80% GC content).

In some instances, the pool of TACS (i.e., each member within each family of TACS) may be chosen so as to define a different % GC content range, deemed to be more suitable for the assessment of specific genetic abnormalities. Non-limiting examples of various % GC content ranges, can be between 19% and 80%, or between 19% and 79%, or between 19% and 78%, or between 19% and 77%, or between 19% and 76%, or between 19% and 75%, or between 19% and 74%, or between 19% and 73%, or between 19% and 72%, or between 19% and 71%, or between 19% and 70%, or between 19% and 69%, or between 19% and 68%, or between 19% and 67%, or between 19% and 66%, or between 19% and 65%, or between 19% and 64%, or between 19% and 63%, or between 19% and 62%, or between 19% and 61%, or between 19% and 60%, or between 19% and 59%, or between 19% and 58%, or between 19% and 57%, or between 19% and 56%, or between 19% and 55%, or between 19% and 54%, or between 19% and 53%, or between 19% and 52%, or between 19% and 51%, or between 19% and 50%, or between 19% and 49%, or between 19% and 48%, or between 19% and 47%, or between 19% and 46%, or between 19% and 45%, or between 19% and 44%, or between 19% and 43%, or between 19% and 42%, or between 19% and 41%, or between 19% and 40%.

As described in further detail below with respect to one embodiment of the data analysis, following amplification and sequencing of the enriched sequences, the test loci and reference loci can then be “matched” or grouped together according to their % GC content (e.g., test loci with a % GC content of 40% is matched with reference loci with a % GC content of 40%). It is appreciated that the % GC content matching procedure may allow slight variation in the allowed matched % GC range. A non-limiting instance, and with reference to the previously described example in text, a test locus with % GC content of 40% could be matched with reference loci of % GC ranging from 39-41%, thereby encompassing the test locus % GC within a suitable range.

To prepare a pool of TACS having the optimized criteria set forth above with respect to size, placement within the human genome and % GC content, both manual and computerized analysis methods known in the art can be applied to the analysis of the human reference genome. In one embodiment, a semi-automatic method is implemented where regions are firstly manually designed based on the human reference genome build 19 (hg19) ensuring that, if applicable, the aforementioned repetitive regions are avoided and subsequently are curated for GC-content using software that computes the % GC-content of each region based on its coordinates on the human reference genome build 19 (hg19). In another embodiment, custom-built software is used to analyses the human reference genome in order to identify suitable TACS regions that fulfill certain criteria, such as but not limited to, % GC content, proximity to repetitive regions and/or proximity to other TACS.

The number of TACS in the pool has been carefully examined and adjusted to achieve the best balance between result robustness and assay cost/throughput. The pool typically contains at least 800 or more TACS, but can include more, such as 1500 or more TACS, 2000 or more TACS or 2500 or more TACS or 3500 or more TACS or 5000 or more TACS. It has been found that an optimal number of TACS in the pool is 5000. It will be appreciated by the ordinarily skilled artisan that a slight variation in pool size typically can be used without altering the results (e.g., the addition or removal of a small number of TACS); accordingly, the number sizes of the pool given herein are to be considered “about” or “approximate”, allowing for some slight variation (e.g., 1-5%) in size. Thus, for example, a pool size of “1600 sequences” is intended to refer to “about 1600 sequences” or “approximately 1600 sequences”, such that, for example, 1590 or 1610 sequences is also encompassed.

In view of the foregoing, in another aspect, the invention provides a method for preparing a pool of TACS for use in the method of the invention for detecting risk of a chromosomal and/or other genetic abnormality, wherein the method for preparing the pool of TACS comprises: selecting regions in one or more chromosomes of interest having the criteria set forth above (e.g., at least 50 base pairs away on either end from the aforementioned repetitive sequences and a GC content of between 19% and 80%, as determined by calculating the GC content of each member within each family of TACS), preparing primers that amplify sequences that hybridize to the selected regions, and amplifying the sequences, wherein each sequence is 100-500 base pairs in length.

For use in the methods of the disclosure, the pool of TACS typically is fixed to a solid support, such as beads (such as magnetic beads) or a column. In one embodiment, the pool of TACS are labeled with biotin and are bound to magnetic beads coated with a biotin-binding substance, such as streptavidin or avidin, to thereby fix the pool of TACS to a solid support. Other suitable binding systems for fixing the pool of TACS to a solid support (such as beads or column) are known to the skilled artisan and readily available in the art. When magnetic beads are used as the solid support, sequences that bind to the TACS affixed to the beads can be separated magnetically from those sequences that do not bind to the TACS.

### Families of TACS

In one embodiment, the pool of TACS comprises a plurality of TACS families directed to different tumor biomarker sequences of interest. Each TACS family comprises a plurality of members that bind to the same tumor biomarker sequence of interest but having different start and/or stop positions with respect to a reference coordinate system for the genomic sequence of interest. Typically, the reference coordinate system that is used for analyzing human genomic DNA is the human reference genome built hg19, which is publically available in the art, but other coordinate systems may also be used. Alternatively, the reference coordinate system can be an artificially created genome based on publically available coordinate systems, such as for example built hg19 of the human genome, that contains only the genomic sequences of interest. Exemplary non-limiting examples of start/stop positions for TACS that bind to chromosome 13, 18, 21, X or Y are shown in FIG. 2.

Each TACS family comprises at least 2 members that bind to the same genomic sequence of interest. In various embodiments, each TACS family comprises at least 2 member sequences, or at least 3 member sequences, or at least 4 member sequences, or at least 5 member sequences, or at least 6 member sequences, or at least 7 member sequences, or at least 8 member sequence, or at least 9 member sequences, or at least 10 member sequences. In various embodiments, each TACS family comprises 2 member sequences, or 3 member sequences, or 4 member sequences, or 5 member sequences, or 6 member sequences, or 7 member sequences, or 8 member sequences, or 9 member sequences, or 10 member sequences. In various embodiments, the plurality of TACS families comprises different families having different numbers of member sequences. For example, a pool of TACS can comprise one TACS family that comprises 3 member sequences, another TACS family that comprises 4 member sequences, and yet another TACS family that comprises 5 member sequences, and the like. In one embodiment, a TACS family comprises 3-5 member sequences. In another embodiment, the TACS family comprises 4 member sequences.

The pool of TACS comprises a plurality of TACS families. Thus, a pool of TACS comprises at least 2 TACS families. In various embodiments, a pool of TACS comprises at least 3 different TACS families, or at least 5 different TACS families, or at least 10 different TACS families, or at least 50 different TACS families, or at least 100 different TACS families, or at least 500 different TACS families, or at least 1000 different TACS families, or at least 2000 TACS families, or at least 4000 TACS families, or at least 5000 TACS families.

Each member within a family of TACS binds to the same genomic region of interest but with different start and/or stop positions, with respect to a reference coordinate system for the genomic sequence of interest, such that the binding pattern of the members of the TACS family is staggered (for example see FIG. 3). In various embodiments, the start and/or stop positions are staggered by at least 3 base pairs, or at least 4 base pairs, or at least 5 base pairs, or at least 6 base pairs, or at least 7 base pairs, or at least 8 base pairs, or at least 9 base pairs, or at least 10 base pairs, or at least 15 base pairs, or at least 20 base pairs, or at least 25 base pairs. Typically, the start and/or stop positions are staggered by 5-10 base pairs. In one embodiment, the start and/or stop positions are staggered by 5 base pairs. In another embodiment, the start and/or stop positions are staggered by 10 base pairs.

### Sample Collection and Preparation

The methods of the invention can be used with a variety of biological samples. Essentially any biological sample containing DNA, and in particular cell-free DNA (cfDNA), can be used as the sample in the methods, allowing for genetic analysis of the DNA therein. For example, a peripheral whole blood sample can be obtained from a subject and plasma can be obtained from the whole blood sample by standard methods. Total cell free DNA can then be extracted from the sample using standard techniques, non-limiting examples of which include a Qiasymphony protocol (Qiagen) suitable for cell free DNA isolation or any other manual or automated extraction method suitable for cell free DNA isolation.

For tumor biomarker detection, the sample is a biological sample obtained from a patient having or suspected of having a tumor. In one embodiment, the DNA sample comprises cell free tumor DNA (cftDNA). In one embodiment, the oncology sample is a sample of tissue (e.g., from a tumor biopsy). In another embodiment the sample is a patient's urine, sputum,ascites, cerebrospinal fluid or pleural effusion. In another embodiment, the oncology sample is a patient plasma sample, prepared from patient peripheral blood. Thus, the sample can be a liquid biopsy sample that is obtained non-invasively from a patient's blood sample, thereby potentially allowing for early detection of cancer prior to development of a detectable or palpable tumor, or can be from a tissue that has or is suspected of having cancer. In another embodiment, the oncology sample is a patient's healthy tissue such as buffy coat, prepared from patient peripheral blood, or buccal swab or healthy tissue adjacent to the tumor or another source of healthy cells. Thus, the healthy cells can provide a source of DNA that allows for detection of germline mutations and comparison with tumor DNA.

For the biological sample preparation, typically cells are lysed and DNA is extracted using standard techniques known in the art, a non-limiting example of which is the Qiagen DNeasy Blood and Tissue protocol. In another embodiment, cell free DNA is isolated from plasma using standard techniques, a non-limiting example of which is the Qiasymphony (Qiagen) protocol.

Following isolation, the cell free DNA of the sample is used for sequencing library construction to make the sample compatible with a downstream sequencing technology, such as Next Generation Sequencing. Typically this involves ligation of adapters onto the ends of the cell free DNA fragments, followed by amplification. Sequencing library preparation kits are commercially available. A non-limiting exemplary protocol for sequencing library preparation is described in detail in Example 1. In another embodiment, nuclear DNA (a non-limiting example of which is DNA extracted from tissue of buffy coat) is fragmented using standard techniques. A non-limiting example of DNA fragmentation is sonication. Fragmented nuclear DNA is then subjected to the same downstream procedures for cell free DNA described in this paragraph.

### Enrichment by TACS Hybridization

The region(s) of interest on the chromosome(s) of interest (e.g., tumor biomarker sequences) is enriched by hybridizing the pool of TACS to the sequencing library, followed by isolation of those sequences within the sequencing library that bind to the TACS. To facilitate isolation of the desired, enriched sequences, typically the TACS sequences are modified in such a way that sequences that hybridize to the TACS can be separated from sequences that do not hybridize to the TACS. Typically, this is achieved by fixing the TACS to a solid support. This allows for physical separation of those sequences that bind the TACS from those sequences that do not bind the TACS. For example, each sequence within the pool of TACS can be labeled with biotin and the pool can then be bound to beads coated with a biotin-binding substance, such as streptavidin or avidin. In a preferred embodiment, the TACS are labeled with biotin and bound to streptavidin-coated magnetic beads. The ordinarily skilled artisan will appreciate, however, that other affinity binding systems are known in the art and can be used instead of biotin-streptavidin/avidin. For example, an antibody-based system can be used in which the TACS are labeled with an antigen and then bound to antibody-coated beads. Moreover, the TACS can incorporate on one end a sequence tag and can be bound to a solid support via a complementary sequence on the solid support that hybridizes to the sequence tag. Furthermore in addition to magnetic beads, other types of solid supports can be used, such as polymer beads and the like.

In certain embodiments, the members of the sequencing library that bind to the pool of TACS are fully complementary to the TACS. In other embodiments, the members of the sequencing library that bind to the pool of TACS are partially complementary to the TACS. For example, in certain circumstances it may be desirable to utilize and analyze data that are from DNA fragments that are products of the enrichment process but that do not necessarily belong to the genomic regions of interest (i.e. such DNA fragments could bind to the TACS because of part homologies (partial complementarity) with the TACS and when sequenced would produce very low coverage throughout the genome in non-TACS coordinates).

Following enrichment of the sequence(s) of interest using the TACS, thereby forming an enriched library, the members of the enriched library are eluted from the solid support and are amplified and sequenced using standard methods known in the art. Next Generation Sequencing is typically used, although other sequencing technologies can also be employed, which provides very accurate counting in addition to sequence information. To detect genetic abnormalities, such as but not limited to, aneuploidies or structural copy number changes requires very accurate counting and NGS is a type of technology that enables very accurate counting. Accordingly, for the detection of genetic abnormalities, such as but not limited to, aneuploidies or structural copy number changes, other accurate counting methods, such as digital PCR and microarrays can also be used instead of NGS. Non-limiting exemplary protocols for amplification and sequencing of the enriched library are described in detail in Example 3.

### Data Analysis

The information obtained from the sequencing of the enriched library can be analyzed using an innovative biomathematical/biostatistical data analysis pipeline. Details of an exemplary analysis using this pipeline are described in depth in Example 4, and in further detail below. Alternative data analysis approaches for different purposes are also provided herein. For example, data analysis approaches for analyzing oncology samples are described in detail in Example 6-9 and in the oncology section below.

The analysis pipeline described in Example 4 exploits the characteristics of the TACS, and the high-efficiency of the target capture enables efficient detection of aneuploidies or structural copy number changes, as well as other types of genetic abnormalities. In the analysis, first the sample's sequenced DNA fragments are aligned to the human reference genome. QC metrics are used to inspect the aligned sample's properties and decide whether the sample is suitable to undergo classification. These QC metrics can include, but are not limited to, analysis of the enrichment patterns of the loci of interest, such as for example the overall sequencing depth of the sample, the on-target sequencing output of the sample, TACS performance, GC bias expectation, fraction of interest quantification. For determining the risk of a chromosomal abnormality in the DNA of the sample, an innovative algorithm is applied. The steps of the algorithm include, but are not limited to, removal of inadequately sequenced loci, read-depth and fragment-size information extraction at TACS-specific coordinates, genetic (GC-content) bias alleviation and ploidy status classification.

Ploidy status determination can be achieved using one or more statistical methods, non-limiting examples of which include a t-test method, a bootstrap method, a permutation test and/or a binomial test of proportions and/or segmentation-based methods and/or combinations thereof. It will be appreciated by the ordinarily skilled artisan that the selection and application of tests to be included in ploidy status determination is based on the number of data points available. As such, the suitability of each test is determined by various factors such as, but not limited to, the number of TACS utilized and the respective application for GC bias alleviation, if applicable. Thus, the aforementioned methods are to be taken as examples of the types of statistical analysis that may be employed and are not the only methods suitable for the determination of ploidy status. Typically, the statistical method results in a score value for the mixed sample and risk of the chromosomal abnormality in the DNA is detected when the score value for the mixed sample is above a reference threshold value.

In particular, one aspect of the statistical analysis involves quantifying and alleviating GC-content bias. In addition to the challenge of detecting small signal changes in DNA in the mixed sample, and/or other components of DNA of interest part of a mixed sample (for example, but not limited to, additional or less genetic material from certain chromosomal regions), the sequencing process itself introduces certain biases that can obscure signal detection. One such bias is the preferential sequencing/amplification of genetic regions based on their GC-content. As such, certain detection methods, such as but not limited to, read-depth based methods, need to account for such bias when examining sequencing data. Thus, the bias in the data needs to be quantified and, subsequently, suitable methods are applied to account for it such that genetic context dependencies cannot affect any statistical methods that may be used to quantify genetic abnormality risk.

For example, one method of quantifying the GC-content bias is to use a locally weighted scatterplot smoothing (LOESS) technique on the sequencing data. Each targeted locus may be defined by its sequencing read-depth output and its' GC-content. A line of best fit through these two variables, for a large set of loci, provides an estimate of the expected sequencing read-depth given the GC-content. Once this GC-bias quantification step is completed, the next step is to use this information to account for possible biases in the data. One method is to normalize the read-depth of all loci by their expected read-depth (based on each locus' GC-content). In principle, this unlinks the read-depth data from their genetic context and makes all data comparable. As such, data that are retrieved from different GC-content regions, such as for example, but not limited, to different chromosomes, can now be used in subsequent statistical tests for detection of any abnormalities. Thus, using the LOESS procedure, the GC bias is unlinked from the data prior to statistical testing. In one embodiment, the statistical analysis of the enriched library sequences comprises alleviating GC bias using a LOESS procedure.

In an alternative embodiment, the GC-content bias is quantified and alleviated by grouping together loci of similar (matching) GC-content. Thus, conceptually this method for alleviating GC-content bias comprises of three steps, as follows:
1) identification and calculation of GC-content in the TACS;
2) alleviation/accounting of GC-content bias using various matching/grouping procedures of the TACS; and
3) calculation of risk of any genetic abnormalities that may be present in the fetus utilizing statistical and mathematical methods on datasets produced from step 2.

For the t-test method, the dataset is split into two groups; the test loci and the reference loci. For each group, subsets of groups are created where loci are categorized according to their GC-content as illustrated in a non-limiting example in the sample Table 1 below:

TABLE 1

| GC | Reference loci read-depth | Test loci read-depth |
|---|---|---|
| 40% | x₁⁴⁰, x₂⁴⁰, . . . xₙₓ₄₀⁴⁰ | y₁⁴⁰, y₂⁴⁰, . . . y_{ny40}⁴⁰ |
| 41% | x₁⁴¹, x₂⁴¹, . . . xₙₓ₄₁⁴¹ | y₁⁴¹, y₂⁴¹, . . . y_{ny41}⁴¹ |
| 42% | x₁⁴², x₂⁴², . . . xₙₓ₄₂⁴² | y₁⁴², y₂⁴², . . . y_{ny42}⁴² |
| . . . | . . . | . . . |

It is appreciated by the ordinarily skilled artisan that subgroup creation may involve encompassing a range of appropriate GC-content and/or a subset of loci that are defined by a given GC-content and/or GC-content range. Accordingly, the % GC content given in the non-limiting example of Table 1 are to be considered “about” or “approximate”, allowing for some slight variation (e.g., 1-2%). Thus, for example, a % GC content of “40%” is intended to refer to “about 40%” or “approximately 40%”, such that, for example, “39%-41%” GC-content loci may also be encompassed if deemed appropriate.

Hence, when referring to a particular GC-content it is understood that the reference and test loci subgroups may comprise of any number of loci related to a particular % GC content and/or range.

Subsequently, for each GC-content subgroup, a representative read-depth is calculated. A number of methods may be utilized to choose this such as, but not limited to, the mean, median or mode of each set. Thus, two vectors of representative read-depth are created where one corresponds to the reference loci and the other to the test loci (e.g., Xm, Ym). In one embodiment, the two vectors may be tested against each other to identify significant differences in read-depth. In another embodiment, the difference of the two vectors may be used to assess if there are significant discrepancies between the test and reference loci. The sample is attributed the score of the test.

For statistical analysis using a bootstrap approach, the dataset is split into two groups, the test loci and the reference loci. The GC-content of each locus is then calculated. Then the following procedure is performed:

A random locus is selected from the reference loci; its read-depth and GC-content are recorded. Subsequently, a random locus from the test loci is selected, with the only condition being that its' GC-content is similar to that of the reference locus. Its read-depth is recorded. It is appreciated by the ordinarily skilled artisan that GC-content similarity may encompass a range of suitable GC-content. As such, referral to a specific % GC content may be considered as “approximate” or “proximal” or “within a suitable range” (e.g., 1%-2%) encompassing the specific % GC content under investigation. Thus, a reference-test locus pair of similar GC-content is created. The difference of the reference-test pair is recorded, say E1. The loci are then replaced to their respective groups. This process is repeated until a bootstrap sample of the same size as the number of test TACS present is created. A representative read-depth of the bootstrap sample is estimated, say E_mu, and recorded. A number of methods may be utilized to do so, such as but not limited to, the mean, mode or median value of the vector, and/or multiples thereof.

The process described above is repeated as many times as necessary and a distribution of E_mu is created. The sample is then attributed a score that corresponds to a percentile of this distribution.

For statistical analysis using a permutation test, the dataset is sorted firstly into two groups, the test-loci and the reference loci. For each group, subsets of groups are created, where loci are categorized according to their GC-content similarity (see columns 2 and 3 of the non-limiting sample Table 2 below). The number of loci present in each test subgroup is also recorded. The loci of the test group are utilized to calculate an estimate of the test-group's read-depth, say Yobs. A representative number from each GC-content subgroup may be selected to do so. Any number of methods may be used to provide a read-depth estimate, such as but not limited to, the mean, median or mode of the chosen loci.

TABLE 2

| GC | Reference loci read-depth | Test loci read-depth | test loci | Merging of loci |
|---|---|---|---|---|
| 40% | x₁⁴⁰, x₂⁴⁰, . . . xₙₓ₄₀⁴⁰ | y₁⁴⁰, y₂⁴⁰, . . . y_{ny40}⁴⁰ | ny40 | x₁⁴⁰, . . . xₙₓ₄₀⁴⁰, y₁⁴⁰, . . . y_{ny40}⁴⁰ |
| 41% | x₁⁴¹, x₂⁴¹, . . . xₙₓ₄₁⁴¹ | y₁⁴¹, y₂⁴¹, . . . y_{ny41}⁴¹ | ny41 | x₁⁴¹, . . . xₙₓ₄₁⁴¹, y₁⁴¹, . . . y_{ny41}⁴¹ |
| 42% | x₁⁴², x₂⁴², . . . xₙₓ₄₂⁴² | y₁⁴², y₂⁴², . . . y_{ny42}⁴² | ny42 | x₁⁴², . . . xₙₓ₄₂⁴², y₁⁴², . . . y_{ny42}⁴² |
| . . . | . . . | . . . | . . . | . . . |

A distribution to test Yobs is then built utilizing loci irrespective of their test or reference status as follows. The test and reference loci of each GC-content subgroup (see last column of sample Table 2) are combined to allow for calculation of a new read-depth estimate. From each merged subgroup a number of loci are chosen at random, where this number is upper-bounded by the number of test-loci utilized in the original calculation of Yobs (e.g., for GC content 40%, and in the context of the non-limiting sample Table 2, this number of loci may be in the range [1,ny40]). The new read-depth estimate is calculated from all the chosen loci. The procedure is iterated as many times as necessary in order to build a distribution of observed means. A sample is then attributed a score that corresponds to the position of Yobs in this distribution using a suitable transformation that accounts for the moments of the built distribution. As with the already described methods, it is appreciated that slight variation in % GC content is allowed (e.g., 1%-2%), if deemed appropriate. Hence, reference to a specific GC-content could be taken as “about” or “approximate”, so that for example when referring to a 40% GC-content, loci that are “approximately” or “about” 40% (e.g., 39%-41%) may be utilized in the method.

For statistical analysis using a binomial test of proportions, fragment-sizes aligned to TACS-specific genomic coordinates are used. There is evidence from the literature that specific types of cancer can be characterized by and/or associated with fragments in the plasma having a smaller size than the expected size of fragments originating from healthy tissues (Jiang et al, (2015), Proceedings of the National Academy of Sciences, 112(11), ppE1317-E1325). The same hypothesis holds true for fragments originating from the placenta/fetus. Specifically, it has been shown that fragments of cell free genetic material originating from the placenta tend to be smaller in length when compared to other cell free genetic material (Chan, K. C. (2004) Clin. Chem. 50:88-92). Hence, the statistic of interest is whether the proportion of small-size fragments aligned to a TACS-specific test-region deviates significantly from what is expected when comparing it to the respective proportion of other TACS-specific reference-regions, as this would indicate fetal genetic abnormalities.

Thus, fragment-sizes are assigned into two groups. Sizes related to the test loci are assigned to one group and fragment-sizes related to the reference loci are assigned to the other group. Subsequently, in each group, fragment sizes are distributed into two subgroups, whereby small-size fragments are assigned into one subgroup and all remaining fragments are designated to the remaining subgroup. The last step computes the proportion of small-sized fragments in each group and uses these quantities in a binomial test of proportions. The score of the test is attributed to the sample under investigation.

The final result of a sample may be given by combining one or more scores derived from the different statistical methods, non-limiting examples of which are given in Example 4.

For statistical analysis using segmentation methods, the read-depth and sequence composition of non-overlapping genomic regions of interest of fixed-size is obtained. On the obtained dataset, GC-content read-depth bias alleviation may be performed, but is not limited to, using a local polynomial fitting method in order to estimate the expected read-depth of regions based on their GC content. The expected value, dependent on GC-content, is then used to normalize regions using suitable methods known to those skilled in the art. The normalized dataset is subsequently processed using one or more segmentation-based classification routines. To do so the algorithms process consecutive data points to detect the presence of read-depth deviations which manifest in the form of a “jump/drop” from their surrounding data points. Depending on the segmentation routine used, data points are given a score which is used towards assigning membership into segments of similar performing read-depths. For example, consecutive data points with score values within a suitable range may be classified as one segment, whereas consecutive data points with score values which exceed the set thresholds may be assigned to a different segment.

### Kits of the Invention

In another aspect, the invention provides kits for carrying out the methods of the disclosure. In one embodiment, the kit comprises a container consisting of the pool of TACS and instructions for performing the method. In one embodiment, the TACS are provided in a form that allows them to be bound to a solid support, such as biotinylated TACS. In another embodiment, the TACS are provided together with a solid support, such as biotinylated TACS provided together with streptavidin-coated magnetic beads.

In one embodiment, the kit comprises a container comprising the pool of TACS and instructions for performing the method, wherein the pool of TACS comprises a plurality of TACS families, wherein each TACS family comprises a plurality of member sequences, wherein each member sequence binds to the same genomic sequence of interest (e.g., tumor biomarker sequence of interest) but has different start and/or stop positions with respect to a reference coordinate system for the genomic sequence of interest, and further wherein:
(i) each member sequence within each TACS family is between 100-500 base pairs in length, each member sequence having a 5′ end and a 3′ end;
(ii) preferably each member sequence binds to the same genomic sequence of interest, and if applicable at least 50 base pairs away, on both the 5′ end and the 3′ end, from regions harboring Copy Number Variations (CNVs), Segmental duplications or repetitive DNA elements; and
(iii) the GC content of the pool of TACS is between 19% and 80%, as determined by calculating the GC content of each member within each family of TACS.

Furthermore, any of the various features described herein with respect to the design and structure of the TACS can be incorporated into the TACS that are included in the kit.

In various other embodiments, the kit can comprise additional components for carrying out other aspects of the method. For example, in addition to the pool of TACS, the kit can comprise one or more of the following (i) one or more components for isolating cell free DNA or nucleated DNA from a biological sample (e.g., as described in Example 1);
(ii) one or more components for preparing the sequencing library (e.g., primers, adapters, buffers, linkers, restriction enzymes, ligation enzymes, polymerase enzymes and the like as described in detail in Example 1); (iii) one or more components for amplifying and/or sequencing the enriched library (e.g., as described in Example 3); and/or (iv) software for performing statistical analysis (e.g., as described in Examples 4 and 6-11).

### Oncology Uses

In various embodiments, the TACS-based enrichment method of the disclosure can be used for a variety of purposes in the oncology field. As described in detail in Examples 6-9, the method allows for detection of tumor biomarkers (including cancer related-germline mutations) in biological samples. The method can be applied to the analysis of essentially any known tumor biomarker. An extensive catalogue of known cancer-associated mutations is known in the art, referred to as COSMIC (Catalogue of Somatic Mutations in Cancer), described in, for example, Forbes, S. A. et al. (2016) Curr. Protocol Hum. Genetic 91:10.11.1-10.11.37; Forbes, S. A. et al. (2017) Nucl. Acids Res. 45:D777-D783; and Prior et al. (2012) Cancer Res. 72:2457-2467. The COSMIC database is publically available at www.cancer.sanger.ac.uk. The database includes oncogenes that have been associated with cancers, any of which can be analyzed using the method of the disclosure. In addition to the COSMIC catalogue, other compilations of tumor biomarker mutations have been described in the art, non limiting examples of which include the ENCODE Project, which describes mutations in the regulatory sites of oncogenes (see e.g., Shar, N. A. et al. (2016) Mol. Canc. 15:76) and ClinVar, a National Center for Biotechnology Information (NCBI) database for genomic variations associated with human health. The Clin Var database is publicly available at www.ncbi.nlm.nih.gov/clinvar.

The methods of the invention can be used to simultaneously analyze a large panel of tumor biomarkers in a single biological sample. For example, in various embodiments, the pool of TACS used in the method detects at least 5, or at least 10, or at least 15, or at least 20, or at least 25, or at least 30, or at least 35, or at least 40, or at least 45 or at least 50 different tumor biomarkers.

For detection of tumor biomarkers, TACS are designed based on the design criteria described herein and the known sequences of tumor biomarker genes and genetic mutations therein associated with cancer. In one embodiment, a plurality of TACS families used in the method bind to a plurality of tumor biomarker sequences of interest selected from the group comprising of ABL, AKT, AKT1, ALK, APC, AR, ARAF, ATM, BAP1, BARD1, BCL, BMPR1A, BRAF, BRCA, BRCA1, BRCA2, BRIP1, CDH1, CDKN, CHEK2, CTNNB1, DDB2, DDR2, DICER1, EGFR, EPCAM, ErbB, ErcC, ESR1, FANCA, FANCB, FANCC, FANCD2, FANCE, FANCF, FANCG, FANCI, FANCL, FANCM, FBXW7, FGFR, FLT, FLT3, FOXA1, FOXL2, GATA3, GNA11, GNAQ, GNAS, GREM1, HOX, HOXB13, HRAS, IDH1, JAK, JAK2, KEAP1, KIT, KRAS, MAP2Ks, MAP3Ks, MET, MLH1, MPL, MRE11A, MSH2, MSH6, MTOR, MUTYH, NBN, NPM1, NRAS, NTRK1, PALB2, PDGFRs, PI3KCs, PMS2, POLD1, POLE, POLH, PTEN, RAD50, RAD51C, RAD51D, RAF1, RB1, RET, RUNX1, SLX4, SMAD, SMAD4, SMARCA4, SPOP, STAT, STK11, TP53, VHL, XPA and XPC, and combinations thereof.

In one embodiment, the plurality of TACS families used in the method bind to a plurality of tumor biomarker sequences of interest selected from the group consisting of, but not limited to, EGFR_6240, KRAS_521, EGFR_6225, NRAS_578, NRAS_580, PIK3CA_763, EGFR_13553, EGFR_18430, BRAF_476, KIT_1314, NRAS_584, EGFR_12378, and combinations thereof.

Representative, exemplary and non-limiting examples of chromosomal start and stop positions for amplifying TACS that bind to exemplary, non-limiting tumor biomarker genes are shown in FIG. 10, for NRAS on chromosome 1, for PI3KCA on chromosome 3, for EGFR on chromosome 7 and for KRAS on chromosome 12. Alternative suitable chromosomal start and stop positions, for these oncogenes and/or for other oncogenes, for amplifying TACS are readily identifiable by one of ordinary skill in the art based on the teachings herein.

In one embodiment of the method, following sequencing of the library preparation and enrichment for the sequences of interest through TACS hybridization, the subsequent step of amplifying the enriched library is performed in the presence of blocking sequences that inhibit amplification of wild-type sequences. Thus, amplification is biased toward amplification of the mutant tumor biomarker sequences.

The pool of TACS and families of TACS used in the method of detecting tumor biomarkers can include any of the design features described herein with respect to the design of the TACS. For example, in various embodiments, each TACS family comprises at least 2, at least 3, at least 4 or at least 5 different member sequences. In one embodiment, each TACS family comprises 4 different member sequences. In various embodiments, the start and/or stop positions for the member sequences within a TACS family, with respect to a reference coordinate system for the genomic sequence of interest, are staggered by at least 5 base pairs, or at least 10 base pairs, or by 5-10 base pairs. In various embodiments, the pool of TACS comprises at least 5, or at least 10 or at least 50 or at least 100 different TACS families, or more.

Suitable statistical analysis approaches for use with oncology samples and detection of tumor biomarkers are described further in Examples 6-9.

The method for detecting tumor biomarkers can be used in a variety of different clinical circumstances in the oncology field. For example, the method can be used for making an initial cancer diagnosis in a subject suspected of having cancer. Accordingly in one embodiment, the method further comprises making a diagnosis of the subject based on detection of at least one tumor biomarker sequence.

Additionally, the method can be used to select an appropriate treatment regimen for a patient diagnosed with cancer, wherein the treatment regimen is designed to be effective against a tumor having the tumor biomarkers detected in the patient's tumor (i.e., known in the art as personalized medicine). Accordingly, in another embodiment, the method further comprises selecting a therapeutic regimen for the subject based on detection of at least one tumor biomarker sequence.

Still further, the method can be used to monitor the efficacy of a therapeutic regiment, wherein changes in tumor biomarker detection are used as an indicator of treatment efficacy. Accordingly, in another embodiment, the method further comprises monitoring treatment efficacy of a therapeutic regimen in the subject based on detection of at least one tumor biomarker sequence.

Moreover, the method can be used to detect relapse and minimal residual disease (MRD), wherein detection of at least one tumor biomarker are used as an indicator of remaining tumor cells in a patient after treatment or tumor recurrence. Accordingly in another embodiment, the method further informs of MRD and disease relapse.

Also, the method can be used to detect cancer-related germline (hereditary) mutations in patients with cancer or individuals suspected of a cancer pre-disposing syndrome wherein detection of at least one germline mutation is used as an indicator for having a cancer pre-disposing syndrome. Accordingly, in another embodiment, the method further comprises diagnosing a patient or an individual with a hereditary cancer pre-disposing syndrome that can inform the clinician to allow for early medical intervention, treatment selection and close monitoring.

### Fragment-Based Analysis

In another aspect, the invention pertains to fragment based analysis of samples, described further in Example 9. There is evidence from the literature that specific types of cancer can be characterized by and/or associated with fragments in the plasma having a smaller size than the expected size of fragments originating from healthy tissues (Jiang et al, (2015), Proceedings of the National Academy of Sciences, 112(11), ppE1317-E1325). The same hypothesis holds true for fragments originating from the placenta/fetus. Specifically, placenta derived fragments are generally of smaller size when compared to fragments originating from maternal tissues/cells. Accordingly, a fragment size-based test was developed and assessed, demonstrating its ability to identify samples harboring chromosomal abnormalities.

Thus, the fragments-based detection may be used to detect abnormalities in mixed samples with low signal-to-noise ratio (e.g., as is the case in detection of cancer).

Accordingly, in one embodiment, a fragments-based test is utilized to detect the presence of somatic copy number aberrations in a sample from a patient suspected of having cancer. For example, a binomial test of proportions, as described Example 4 and Example 9, can be used for the detection of increased presence of nucleic acid material originating from non-healthy tissue (e.g., tumor tissue) based on fragment size. In particular, under the null hypothesis that the distribution of fragment sizes originating from both healthy and cancerous cells is the same, a binomial test for proportions (as described in Example 4 and Example 9) using continuity correction can be utilized to quantify any evidence against it.

### EXAMPLES

The present invention is further illustrated by the following examples, which should not be construed as further limiting. The contents of all references, appendices, Genbank entries, patents and published patent applications cited throughout this application are expressly incorporated herein by reference in their entirety.

### Example 1: Sample Collection and Library Preparation

The general methodology for the TACS-based multiplexed parallel analysis approach for genetic assessment is shown schematically in FIG. 1. In this example, methods for collecting and processing a maternal plasma sample (containing maternal and fetal DNA), followed by sequencing library preparation for use in the methodology of FIG. 1 are described. The DNA sample and library preparation described herein can similarly be used with DNA samples from tumors for tumor biomarker detection (see Example 6-9).

### Sample Collection

Plasma samples were obtained anonymously from pregnant women after the 10^{th} week of gestation. Protocols used for collecting samples for our study were approved by the Cyprus National Bioethics Committee, and informed consent was obtained from all participants.

### Sample Extraction

Cell Free DNA was extracted from 2-4 ml plasma from each individual using a manual or automated extraction method suitable for cell free DNA isolation such as for example, but not limited to, Qiasymphony protocol suitable for cell free fetal DNA isolation (Qiagen) (Koumbaris, G. et al. (2016) Clinical chemistry, 62(6), pp.848-855).

### Sequencing Library Preparation

Extracted DNA from maternal plasma samples was used for sequencing library construction. Standard library preparation methods were used with the following modifications. A negative control extraction library was prepared separately to monitor any contamination introduced during the experiment. During this step, 5′ and 3′ overhangs were filled-in, by adding 12 units of T4 polymerase (NEB) while 5′ phosphates were attached using 40 units of T4 polynucleotide kinase (NEB) in a 100 μl reaction and subsequent incubation at 25° C. for 15 minutes and then 12° C. for 15 minutes. Reaction products were purified using the MinElute kit (Qiagen). Subsequently, adaptors P5 and P7 (see adaptor preparation) were ligated at 1:10 dilution to both ends of the DNA using 5 units of T4 DNA ligase (NEB) in a 40 μl reaction for 20 minutes at room temperature, followed by purification using the MinElute kit (Qiagen). Nicks were removed in a fill-in reaction with 16 units of Bst polymerase (NEB) in a 40 μl reaction with subsequent incubation at 65° C. for 25 minutes and then 12° C. for 20 minutes. Products were purified using the MinElute kit (Qiagen). Library amplification was performed using a Fusion polymerase (Herculase II Fusion DNA polymerase (Agilent Technologies) or Pfusion High Fidelity Polymerase (NEB)) in 50 μl reactions and with the following cycling conditions, 95° C. for 3 minutes; followed by 10 cycles at 95° C. for 30 seconds, 60° C. for 30 seconds, 72° C. for 30 seconds and finally 72° C. for 3 minutes (Koumbaris, G. et al. (2016) Clinical chemistry, 62(6), pp.848-855). The final library products were purified using the MinElute Purification Kit (Qiagen) and measured by spectrophotometry.

### Adaptor Preparation

Hybridization mixtures for adapter P5 and P7 were prepared separately and incubated for 10 seconds at 95° C. followed by a ramp from 95° C. to 12° C. at a rate of 0.1° C./second. P5 and P7 reactions were combined to obtain a ready-to-use adapter mix (100 μM of each adapter). Hybridization mixtures were prepared as follows: P5 reaction mixture contained adaptor P5_F (500 μM) at a final concentration of 200 μM, adaptor P5+P7_R (500 μM) at a final concentration of 200 μM with 1X oligo hybridization buffer. In addition, P7 reaction mixture contained adaptor P7_F (500 uM) at a final concentration of 200 uM, adapter P5+P7_R (500 μM) at a final concentration of 200 μM with 1X oligo hybridization buffer (Koumbaris, G. et al. (2016) Clinical chemistry, 62(6), pp.848-855.). Sequences were as follows, wherein *=a phosphorothioate bond (PTO) (Integrated DNA Technologies):

adaptor P5_F:
(SEQ ID NO: XX)
A*C*A*C*TCTTTCCCTACACGACGCTCTTCCG*A*T*C*T
adaptor P7_F:
(SEQ ID NO: YY)
G*T*G*A*CTGGAGTTCAGACGTGTGCTCTTCCG*A*T*C*T,
adaptor_P5+P7_R:
(SEQ ID NO: ZZ)
A*G*A*T*CGGAA*G*A*G*C.

### Example 2: TArget Capture Sequences (TACS) Design and Preparation

This example describes preparation of custom TACS for the detection of whole or partial chromosomal abnormalities for chromosomes 13, 18, 21, X, Y or any other chromosome, as well as other genetic abnormalities, such as but not limited to, microdeletion/microduplication syndromes, translocations, inversions, insertions, and other point or small size mutations. The genomic target-loci used for TACS design were selected based on their GC content and their distance from repetitive elements (minimum 50 bp away). TACS size can be variable. In one embodiment of the method the TACS range from 100-500 bp in size and are generated through a PCR-based approach as described below. The TACS were prepared by simplex polymerase chain reaction using standard Taq polymerase, primers designed to amplify the target-loci, and normal DNA used as template. The chromosomal regions used to design primers to amplify suitable loci on chromosomes 13, 18, 21 and X, to thereby prepare the pool of TACS for analysis of chromosomes 13, 18, 21 and X, are shown in FIG. 2.

All custom TACS were generated using the following cycling conditions: 95° C. for 3 minutes; 40 cycles at 95° C. for 15 seconds, 60° C. for 15 seconds, 72° C. for 12 seconds; and 72° C. for 12 seconds, followed by verification via agarose gel electrophoresis and purification using standard PCR clean up kits such as the Qiaquick PCR Purification Kit (Qiagen) or the NucleoSpin 96 PCR clean-up (Mackerey Nagel) or the Agencourt AMPure XP for PCR Purification (Beckman Coulter). Concentration was measured by Nanodrop (Thermo Scientific).

### Example 3: TACS Hybridization and Amplification

This example describes the steps schematically illustrated in FIG. 1 of target capture by hybridization using TACS, followed by quantitation of captured sequences by Next Generation Sequencing (NGS).

### TACS Biotinylation

TACS were prepared for hybridization, as previously described (Koumbaris, G. et al. (2016) Clinical chemistry, 62(6), pp.848-855), starting with blunt ending with the Quick Blunting Kit (NEB) and incubation at room temperature for 30 minutes. Reaction products were subsequently purified using the MinElute kit (Qiagen) and were ligated with a biotin adaptor using the Quick Ligation Kit (NEB) in a 40 μl reaction at RT for 15 minutes. The reaction products were purified with the MinElute kit (Qiagen) and were denatured into single stranded DNA prior to immobilization on streptavidin coated magnetic beads (Invitrogen).

### TACS Hybridization

Amplified libraries were mixed with blocking oligos (Koumbaris, G. et al. (2016) Clinical chemistry, 62(6), pp.848-855) (200 μM), 5 μg of Cot-1 DNA (Invitrogen), 50 μg of Salmon Sperm DNA (Invitrogen), Agilent hybridization buffer 2×, Agilent blocking agent 10×, and were heated at 95° C. for 3 minutes to denature the DNA strands. Denaturation was followed by 30 minute incubation at 37° C. to block repetitive elements and adaptor sequences. The resulting mixture was then added to the biotinylated TACS. All samples were incubated in a rotating incubator for 12-48 hours at 66° C. After incubation, the beads were washed as described previously and DNA was eluted by heating (Koumbaris, G. et al. (2016) Clinical chemistry, 62(6), pp.848-855). Eluted products were amplified using outer-bound adaptor primers. Enriched amplified products were pooled equimolarly and sequenced on a suitable platform.

If appropriate, amplification may be biased toward amplification of specific/desired sequences. In one embodiment of the method, this is performed when amplification is performed in the presence of sequences that hybridize to the undesired sequence of interest, and as such block the action of the polymerase enzyme during the process. Hence, the action of the amplification enzyme is directed toward the sequence of interest during the process.

### Example 4: Bioinformatics Sample Analysis

This example describes representative statistical analysis approaches for use in the methodology illustrated in FIG. 1 (“analysis pipeline” in FIG. 1).

### Human Genome Alignment

For each sample, the bioinformatic pipeline routine described below was applied in order to align the sample's sequenced DNA fragments to the human reference genome. Targeted paired-end read fragments obtained from NGS results were processed to remove adaptor sequences and poor quality reads (Q-score<25) using the cutadapt software (Martin, M. et al. (2011) EMB.netJournal 17.1). The quality of the raw and/or processed reads as well as any descriptive statistics which aid in the assessment of quality check of the sample's sequencing output were obtained using the FastQC software (Babraham Institute (2015) FastQC) and/or other custom-built software. Processed reads which were at least 25 bases long were aligned to the human reference genome built hg19 (UCSC Genome Bioinformatics) using the Burrows-Wheel Alignment algorithm (Li, H. and Durbin, R. (2009) Bioinformatics 25:1754-1760) but other algorithms known to those skilled in the art may be used as well. If relevant, duplicate reads were removed post-alignment. Where applicable, sequencing output pertaining to the same sample but processed on separate sequencing lanes, was merged to a single sequencing output file. The removal of duplicates and merging procedures were performed using the Picard tools software suite (Broad Institute (2015) Picard) and/or the Sambamba tools software suite (Tarasov, Artem, et al. “Sambamba: fast processing of NGS alignment formats.” Bioinformatics 31.12 (2015):2032-2034.). A realignment procedure, using tools known to those in the art, may also be performed.

The above software analysis resulted in a final aligned version of a sequenced sample against the human reference genome and all subsequent steps were based on this aligned version. Information in terms of Short Nucleotide Polymorphisms (SNPs) at loci of interest was obtained using bcftools from the SAMtools software suite (Li, H. et al. (2009) Bioinformatics 25:2078-2079) and/or other software known to those skilled in the art. The read-depth per base, at loci of interest, was obtained using the mpileup option of the SAMtools software suite, from here on referred to as the mpileup file. Information pertaining to the size of the aligned fragments was obtained using the view option of the SAMtools software suite, from here on referred to as the fragment-sizes file and/or other software known to those skilled in the art.

The mpileup file and the fragment-sizes file were processed using custom-build application programming interfaces (APIs) written in the Python and R programming languages (Python Software Foundation (2015) Python; The R Foundation (2015) The R Project for Statistical Computing). The APIs were used to determine the ploidy state of chromosomes of interest, and/or other genetic abnormalities in regions of interest across the human genome, using a series of steps (collectively henceforth referred to as the “algorithm”) and to also collect further descriptive statistics to be used as quality check metrics, such as but not limited to fetal fraction and/or fraction of interest quantification (collectively henceforth referred to as the “QC metrics”). The APIs can also be used for the assessment of genetic abnormalities from data generated when applying the described method in cases of multiple gestation pregnancies, as well as other genetic abnormalities such as, but not limited to, microdeletions, microduplications, copy number variations, translocations, inversions, insertions, point mutations and mutational signatures.

### QC Metrics

QC metrics were used to inspect an aligned sample's properties and decide whether the sample was suitable to undergo classification. These metrics were, but are not limited to:
(a) The enrichment of a sample. The patterns of enrichment are indicative of whether a sample has had adequate enrichment across loci of interest in a particular sequencing experiment (herein referred to as a “run”). To assess this, various metrics are assessed, non-limiting examples of which are:
   (i) overall sample on-target read depth,
   (ii) sample on-target sequencing output with respect to total mapped reads,
   (iii) individual TACS performance in terms of achieved read-depth,
   (iv) kurtosis and skewness of individual TACS enrichment,
   (v) kurtosis and skewness moments that arise from all TACS,
   (vi) fragment size distribution,
   (vii) percentage of duplication,
   (viii) percentage of paired reads and,
   (ix) percentage of aligned reads,
      if applicable.

The above checks are also taken into consideration with regards to GC-bias enrichment. Samples that fail to meet one or more of the criteria given above are flagged for further inspection, prior to classification.
(b) A sample's fetal fraction or fraction of interest. Samples with an estimated fetal fraction, or fraction of interest, that is below a specific threshold are not classified. Furthermore, if applicable the fraction of interest may be calculated using more than one method and concordance of results between estimation methods may be used as an additional QC prior to classification.

### The Algorithm

The algorithm is a collection of data processing, mathematical and statistical model routines arranged as a series of steps. The algorithm's steps aim in deciding the relative ploidy state of a chromosome of interest with respect to all other chromosomes of the sequenced sample and is used for the detection of whole or partial chromosomal abnormalities for chromosomes 13, 18, 21, X, Y or any other chromosome, as well as other genetic abnormalities such as, but not limited to, microdeletion/microduplication syndromes and other point or small size mutations. As such the algorithm can be used, but is not limited to, the detection of whole or partial chromosomal abnormalities for chromosomes 13, 18, 21, X,Y or any other chromosome, as well as other genetic abnormalities such as, but not limited to, microdeletions, microduplications, copy number variations, translocations, inversions, insertions, point mutations and other mutational signatures. The algorithm carries out, but is not limited to, two types of assessments, one pertaining to the read-depth information of each sample and the other to the distribution of fragment-sizes, across TACS-specific regions. One or more statistical tests may be associated with each type of assessment, non-limiting examples of which are given in the statistical methods described herein.

In the case of read-depth associated tests, the algorithm compares sequentially the read-depth of loci from each chromosome of interest (herein referred to as the test chromosome) against the read-depth of all other loci (herein referred to as the reference loci) to classify its ploidy state. For each sample, these steps were, but are not limited to:
(a) Removal of inadequately sequenced loci. The read-depth of each locus was retrieved. Loci that have not achieved a minimum number of reads, were considered as inadequately enriched and were removed prior to subsequent steps.
(b) Genetic (GC-content) bias alleviation. The sequencing procedure may introduce discrepancies in read-depth across the loci of interest depending on their GC content. To account for such bias, a novel sequence-matching approach that increases both sensitivity and specificity to detect chromosomal aneuploidies was employed. The GC content of each locus on the test chromosome was identified and similar genetic loci were grouped together to form genetically matched groups. The procedure was repeated for the reference loci. Then, genetically matched groups from the test chromosome were conditionally paired with their genetically matched group counterparts on the reference chromosome(s). The groups may have any number of members. The conditionally matched groups were then used to assess the ploidy status of test chromosomes.
(c) Genetic abnormality determination. Ploidy status determination, or other genetic abnormalities of interest such as but not limited to microdeletions, microduplications, copy number variations, translocations, inversions, insertions, point mutations and other mutational signatures was achieved using a single statistical method and/or a weighted score approach on the result from the following, but not limited to, statistical methods:

Statistical Method 1: The differences in read-depth of the conditionally paired groups were tested for statistical significance using the t-test formula:

$$t = \frac{\hat{x} - μ}{s / \sqrt{n}}$$

where t is the result of the t-test, \hat{x} is the average of the differences of the conditionally paired groups, μ is the expected read-depth and is set to a value that represents insignificant read-depth differences between the two groups, s the standard deviation of the differences of the conditionally paired groups and n the length of the vector of the conditionally paired differences. The magnitude of the t-score was then used to identify evidence, if any, against the null hypothesis of same ploidy between reference and test chromosomes. Specifically, t>=c1 (where c1 is a predefined threshold belonging to the set of all positive numbers) shows evidence against the null hypothesis of no difference.

Statistical Method 2: Bivariate nonparametric bootstrap. The bootstrap method depends on the relationship between the random variables X (read-depth of reference loci) and Y (read-depth of test loci). Here, the read depth of baits on the reference group (random variable denoted by X) were treated as the independent covariate. The first step of the iterative procedure involved random sampling with replacement (bootstrapping) of the read-depths of loci on the reference chromosomes, i.e. (x1,g1), . . . , (xn,gn), where the parameter g is known and denotes the GC-content of the chosen bait. Then, for each randomly selected reference bait (xi,gi), a corresponding read depth was generated for a genetically matched locus i.e., (y1,g1), . . . , (yn,gn). Thus, the bivariate data (x1,y1), (x2,y2), . . . , (xn,yn) was arrived at, which was conditionally matched on their GC-content (parameter gi). The differences between the read depths of the genetically matched bootstrapped values xi and yi were used to compute the statistic of interest in each iteration. In one embodiment this statistical measure can be, but is not limited to, the mode, mean or median of the recorded differences, and/or multiples thereof. The procedure was repeated as necessary to build up the distribution of the statistic of interest from these differences. The sample was assigned a score that corresponds to a specific percentile of the built distribution (e.g. 5^{th} percentile). Under the null hypothesis the ploidy between chromosomes in the reference and test groups is not different. As such, samples whose score for a particular chromosome, was greater than a predefined threshold, say c2, were classified as statistically unlikely to have the same ploidy. Other statistical measures may be employed.

Statistical Method 3: Stratified permutation test. The statistic of interest is the read-depth estimate of the test chromosome, denoted by Ŷ_{obs}, which is calculated using all loci of the test chromosome's genetically matched groups as follows:

$${\hat{Y}}_{obs} = \frac{{\sum }_{j = 1}^{j = T} {\sum }_{i = 1}^{i = Nj} {y}_{ij}}{{\sum }_{j = 1}^{j = T} Nj}$$

where yᵢⱼ is the read-depth of locus i part of the genetically matched group j (i.e., loci belonging to a specific group based on their GC-content), Nj is the number of test loci part of the genetically matched group j and T the number of genetically matched groups.

Subsequently, a null distribution to test Ŷ_{obs} was built. To do so, for each group j, the test and reference loci were combined (exchangeability under the null hypothesis), and each group j was sampled randomly up to Nj times without replacement (stratified permutation). This created a vector of values, say yi, and from this the vector's average value, say ýᵢ, was calculated. The procedure was repeated as necessary to build the null distribution. Finally Ŷ_{obs}, was studentised against the null distribution using the formula:

$${Z}_{Yobs} = \frac{Y {\hat{}}_{obs} - \hat{Y}}{{σ}_{Y}}$$

where Ŷ and σ_{Y} are the first and square root of the second moment of all permuted ýᵢ statistic values. Samples whose Z_{Yobs} was greater than a predefined threshold, say c3, were statistically less likely to have the same ploidy in the reference and test groups.

In the case of fragment-size associated tests, the algorithm computes the proportion of small-size fragments found in test-loci and compares it with the respective proportion in reference-loci as described in Statistical Method 4 below.

Statistical Method 4: Fragment Size Proportions. For each sample the number and size of fragments aligned onto the human reference genome at the corresponding TACS coordinates, is extracted. The data is subsequently filtered so as to remove fragment-sizes considered statistical outliers using the median outlier detection method. Specifically, outliers are defined as those fragments whose size is above or below the thresholds, Fₜₕᵣ, set by equation:

$${F}_{thr} = {F}_{median} \pm ( X \times IQR )$$

where F_{median} is the median fragment-size of all fragments of a sample, X is a variable that can take values from the set of R+, and IQR is the interquartile range of fragment sizes. Thereafter, a binomial test of proportions is carried out to test for supporting evidence against the null hypothesis, H0, where this is defined as:

H0: The proportion of small fragments of the test-region is not different from the proportion of small-fragments of the reference region.

In various embodiments of the invention, small fragments are defined as those fragments whose size is less than or equal to a subset of Z+, that is upper-bounded by 160 bp. If the set of all TACS are defined as T, then the test region can be any proper subset S which defines the region under investigation, and the reference region is the relative complement of S in T. For example, in one embodiment of the invention, the set S is defined by all TACS-captured sequences of chromosome 21 and thus the reference set is defined by all TACS-captured fragments on the reference chromosomes, and/or other reference loci The alternative hypothesis, H1, is defined as:

H1: The proportion of small fragments of the test-region is not equal to the proportion of test fragments of the reference region.

As such, and taking into account continuity correction, the following score is computed (Brown et. al, Harrel):

$${W}_{test} = ( {p}^{′} - {p}_{ref} ) / \sqrt{\frac{{p}^{′} ( 1 - {p}^{′} )}{{N}_{test}}}$$

$$where$$

$${p}^{′} = \frac{( {F}^{′} + 0.5 )}{( {N}_{test} + 1 )}$$

$${p}_{ref} = \frac{( {F}_{ref} + 0.5 )}{( {N}_{ref} + 1 )}$$

\acute{F} is the number of small-size fragments on the test-region, F_{ref} the number of small size fragments on the reference region, Nₜₑₛₜ the number of all fragments on the test region and N_{ref} the number of all fragments on the reference region.

For each sample, the algorithm tests sequentially the proportion of fragment sizes of regions under investigation (for example, but not limited to, chromosome 21, chromosome 18, chromosome 13 or other (sub)chromosomal regions of interest) against reference regions; those not under investigation at the time of testing. For each sample a score is assigned for each test. Scores above a set-threshold, say c4, provide evidence against the null hypothesis.

Weighted Score method 1: In one embodiment of the method, a weighted score was attributed to each sample s, computed as a weighted sum of all statistical methods using the formula:

$${V}_{s} ( R , F ) = {z}_{1} max \{ {R}_{s} , {F}_{s} \} + ( 1 - {z}_{1} ) min \{ {R}_{s} , {F}_{s} \}$$

where Rₛ is the run-specific corrected score arising from a weighted contribution of each read-depth related statistical method for sample s and is defined as:

$${R}_{s} = \frac{( {\sum }_{i} {w}_{i} {S}_{is} - {{R}^{′}}_{r} )}{{σ}_{r}}$$

and Ŕᵣ is the run-specific median value calculated from the vector of all unadjusted read-depth related weighted scores that arise from a single sequencing run, and σᵣ is a multiple of the standard deviation of R scores calculated from a reference set of 100 euploid samples. The terms max{Rₛ, Fₛ} and min{Rₛ, Fₛ} denote the maximum and minimum values of the bracketed set, respectively.

Fₛ is the run-specific corrected score arising from the fragment-size related statistical method and is defined as:

$${F}_{s} = \frac{( {W}_{test} - {{R}^{′}}_{f} )}{{σ}_{f}}$$

where Wₜₑₛₜ is as defined earlier, Ŕ_{f} is the run specific median calculated from the vector of all unadjusted fragment-related statistical scores that arise from a single sequencing run, and σ_{f} is a multiple of the standard deviation of F scores calculated from a reference set of 100 euploid samples.

A unique classification score of less than a predefined value indicates that there is no evidence from the observed data that a sample has a significant risk of aneuploidy.

Weighted Score method 2: In another embodiment of the method, the weighted score arising from the statistical methods described above was used to assign each sample a unique genetic abnormality risk score using the formula:

$$R ( t , c ) = {\sum }_{j = 0}^{j = N} {w}_{j} \frac{{t}_{j}}{{c}_{j}}$$

where R is the weighted score result, wⱼ the weight assigned to method j, tⱼ the observed score resulting from method j, and cⱼ the threshold of method j.

A unique classification score of less than a predefined value indicates that there is no evidence from the observed data that a sample has a significant risk of aneuploidy.

Since all read depths from baits in the reference group were assumed to be generated from the same population, and in order to have a universal threshold, run-specific adjustments were also employed to alleviate run-specific biases.

The aforementioned method(s), are also suitable for the detection of other genetic abnormalities, such as but not limited to, subchromosomal abnormalities. A non-limiting example is the contiguous partial loss of chromosomal material leading to a state of microdeletion, or the contiguous partial gain of chromosomal material leading to a state of microduplication. A known genetic locus subject to both such abnormalities is 7q11.23. In one embodiment of statistical method 1, synthetic plasma samples of 5%, 10% and 20% fetal material were tested for increased risk of microdeletion and/or microduplication states for the genetic locus 7q11.23.

For point mutations various binomial tests are carried out that take into consideration the fetal fraction estimate of the sample, f, the read-depth of the minor allele, r, and the total read-depth of the sequenced base, n. Two frequent, yet non-limiting examples involve assessment of the risk when the genetic abnormality is a recessive point mutation or a dominant point mutation.

In the non-limiting example of a recessive point mutation the null hypothesis tested is that both the mother and the fetus are heterozygous (minor allele frequency is 0.5) against the alternative in which the fetus is homozygous (minor allele frequency is 0.5−f/2). A small p-value from the corresponding likelihood ratio test would indicate evidence against the null. In the non-limiting example of a dominant point mutation the null hypothesis tested is that the mother and fetus are homozygous at the given position against the alternative in which only the fetus is heterozygous for the given position. A small p-value from the corresponding likelihood ratio test would indicate evidence against the null.

In addition to the above, fetal sex determination methods were also developed, with non-limiting examples given below. In one embodiment of the invention, fetal sex was assigned to a sample using a Poisson test using the formula:

$$Pr ( {r}_{y} \leq k ) = {e}^{- λ} {\sum }_{i = 0}^{i = k} \frac{{λ}^{i}}{i !}$$

$$where$$

$$λ = \frac{fB μ}{2}$$

and f is the fetal fraction estimate of the sample, B is the number of target sequences on chromosome Y, μ is the read-depth of the sample and k is the sum of reads obtained from all targets B. The null hypothesis of the Poisson test was that the sample is male. A value of Pr(r_{y}) less than a threshold c_{y} was considered as enough evidence to reject the null hypothesis, i.e. the sample is not male. If any of the terms for computing Pr(r_{y}) were unavailable, then the sample's sex was classified as NA (not available).

In another embodiment of the invention, fetal sex was assigned using the average read-depth of target sequences on chromosome Y. If the average read-depth of the target-sequences was over a predefined threshold, where such threshold may be defined using other sample-specific characteristics such as read-depth and fetal-fraction estimate, the fetal sex was classified as male. If the average read-depth was below such threshold then the sample was classified as female.

### Fetal Fraction Estimation/Fraction of Interest Estimation

Several methods have been developed to estimate fetal fraction that can be applied to singleton and/or to multiple gestation pregnancies. As such, and dependent on the type of pregnancy, the fetal fraction estimate can be obtained from either method or as a weighted estimate from a subset and/or all developed methods. Some non-limiting examples are given below.

In one embodiment, a machine learning technique has been developed based on Bayesian inference to compute the posterior distribution of fetal DNA fraction using allelic counts at heterozygous loci in maternal plasma of singleton pregnancies. Three possible informative combinations of maternal/fetal genotypes were utilized within the model to identify those fetal DNA fraction values that get most of the support from the observed data.

Let f denote the fetal DNA fraction. If the mother is heterozygous at a given genomic locus, the fetal genotype can be either heterozygous or homozygous resulting in expected minor allele frequencies at 0.5 and 0.5−f/2, respectively. If the mother is homozygous and the fetus is heterozygous then the expected minor allele frequency will be f/2. A Markov chain Monte Carlo method (a Metropolis-Hastings algorithm) (The R Foundation (2015) The R Project for Statistical Computing) was used with either a non-informative or an informative prior (i.e. incorporate additional information such as gestational age, maternal weight etc.) to obtain a sequence of random samples from the posterior probability distribution of fetal DNA fraction that is based on a finite mixture model.

In another embodiment, the fetal fraction estimate is computed only from the fetus-specific minor allele frequency (MAF) cluster, i.e. the cluster formed when the mother is homozygous and the fetus is heterozygous for a given genomic locus. It is assumed that the mean value of the fetal fraction estimate is normally distributed as N (2\acute{x}, σ_{\acute{x}}), where \acute{x} is the mean of the fetus-specific MAF, and σ_{\acute{x}} is the standard deviation of the fetus-specific MAF. The fetal fraction estimate is then obtained from percentiles of the computed distribution, N(2\acute{x}, σ_{\acute{x}}).

For multiple gestation pregnancies, non-limiting examples of which include monozygotic and dizygotic twin pregnancies, triplet pregnancies and various egg and/or sperm donor cases, the fetal fraction can be estimated using information obtained from heterozygous genetic loci whose MAF value is less than a threshold, say Mₜₕᵣₑₛₕ, and derived from potential fetus-specific SNPs. The ordinarily skilled artisan will appreciate that fetus specific SNPs can originate from any fetus, or from any possible combination of the fetuses or from all the fetuses of the gestation. As such, an algorithm that estimates the fetal fraction of the fetus with the smallest contribution to the total fetal content, by taking into account the combinatorial contribution of each fetus to the MAF values that define fetus-specific SNPs, and also allows for inhomogeneous contribution of fetal material to the total fetal content of plasma derived material has been developed. To this effect, a two-step approach is employed by the algorithm.

In one embodiment of the algorithm, the multiple gestation pregnancy under consideration is a dizygotic twin pregnancy. As a first step, the algorithmic implementation of the model utilizes all informative SNPs and allows for inhomogeneous fetal contribution that can be explained with a fold-difference in fetal fraction estimates of a set threshold, say cf. Specifically, if f1 and f2 represent the fetal fractions of fetus one and fetus two, and f1<=f2, then the assumption is that f2<=cf f1, with cf being a positive real number greater than or equal to 1. Under this assumption, the observed data D, defined as counts of the alternate and reference alleles at informative SNP loci, are believed to be generated from a mixture distribution of three Binomials (defined by parameters, f1/2, f2/2 and (f1+f2)/2), with the posterior distribution p(f1,f2|D) being proportional to the observational model which can be written as p(f1|f2,D) p(f2|D). The posterior distribution p(f1,f2|D) is sampled with an MCMC Metropolis-Hastings algorithm using a uniform prior. The empirical quantile approach is performed on the generated data array to infer the fetal fractions.

As a second step, the algorithm runs a model-based clustering algorithm (Finite Gaussian mixture modeling fitted via EM algorithm; R-package: mclust) to identify whether there exists a separate outlier SNP cluster which is believed to be centered around f1/2. Existence of such a cluster with a mean invalidating the cf>=f2/f1 assumption, leads to estimation of f1 using only SNPs part of the identified cluster.

The methods described above are suited to the determination of the fraction of any component of interest part of a mixed sample. As such, the methods are not to be understood as applicable only to the application of fetal fraction estimation and can be applied to the estimation of any component of interest part of a mixed sample, as outlined in Example 6.

### Example 5: Target Enrichment Using Families of TACS

In this example, a family of TACS, containing a plurality of members that all bind to the same target sequence of interest, was used for enrichment, compared to use of a single TACS binding to a target sequence of interest. Each member of the family of TACS bound to the same target sequence of interest but had different start/stop coordinates with respect to a reference coordinate system for that target sequence (e.g., the human reference genome, built hg19). Thus, when aligned to the target sequence, the family of TACS exhibit a staggered binding pattern, as illustrated in FIG. 3. Typically, the members of a TACS family were staggered approximately 5-10 base pairs.

A family of TACS containing four members (i.e., four sequences that bound to the same target sequence but having different start and/or stop positions such that the binding of the members to the target sequence was staggered) was prepared. Single TACS hybridization was also prepared as a control. The TACS were fixed to a solid support by labelling with biotin and binding to magnetic beads coated with a biotin-binding substance (e.g., streptavidin or avidin) as described in Example 3. The family of TACS and single TACS were then hybridized to a sequence library, bound sequences were eluted and amplified, and these enriched amplified products were then pooled equimolarly and sequenced on a suitable sequencing platform, as described in Example 3.

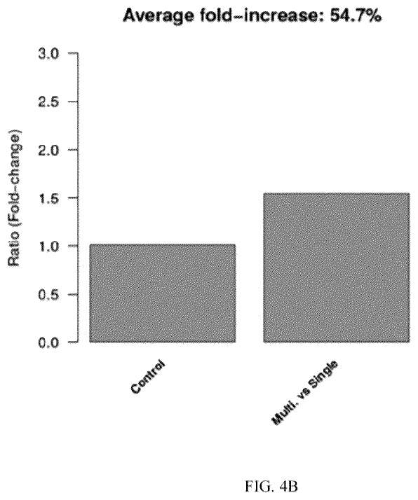

The enriched sequences from the family of TACS sample and the single TACS sample were analyzed for read-depth. The results are shown in FIGS. 4A and 4B. As shown in FIG. 4A, target sequences of interest enriched using the family of four TACS (red dots) exhibited a fold-change in read-depth when compared to control sequences that were subjected to enrichment using only a single TACS (blue dots). Fold-change was assessed by normalizing the read-depth of each locus by the average read-depth of a sample, wherein the average read- depth was calculated from all loci enriched with a single TACS. As shown in FIG. 4B, an overall 54.7% average increase in read-depth was observed using the family of four TACS.

This example demonstrates that use of a family of TACS, as compared to a single TACS, results in significantly improved enrichment of a target sequence of interest resulting in significantly improved read-depth of that sequence.

### Example 6: Tumor Biomarker Detection in Reference Material

In this example, the TACS methodology, illustrated in FIG. 1, was used for the detection of tumor biomarkers in certified reference material known to harbor particular genetic mutations that are tumor biomarkers. For detection of the tumor biomarker sequences of interest, families of TACS, as described in Example 5, were used.

A sample of certified reference material harboring known tumor-associated genetic mutations was commercially obtained and samples were prepared to simulate tumor loads of 0.1%, 1.0% and 5.0%.

The samples were subjected to the TACS methodology illustrated in FIG. 1 using families of TACS that bound to the following tumor-associated genetic mutations: EGFR_6240, KRAS_521, EGFR_6225, NRAS_578, NRAS_580, PIK3CA_763, EGFR_13553, EGFR_18430.

Following amplification and sequence of the TACS-enriched products, data analysis was performed as follows. Sequencing products were processed to remove adaptor sequences and poor quality reads. Reads whose length was at least 25 bases long post adaptor-removal were aligned to either:
(a) the human reference genome built hg19, or
(b) an artificially created genome based on built hg19 which contains only sequences of interest.

If relevant, duplicate reads were removed post-alignment. Where applicable, sequencing output pertaining to the same sample but processed on separate sequencing lanes was merged to a single sequencing output file. Local realignment of the data, using tools known in the art, may also be performed. The above software analysis provided a final aligned version of a sequenced sample against the reference genome, defined here as the final BAM file, where information can be extracted from it in terms of Short Nucleotide Polymorphisms (SNPs), Single Nucleotide Variants (SNVs) and other genetic variations with respect to a reference sequence at loci of interest, read-depth per base and the size of aligned fragments. Various available tools known to those skilled in the art, such as but not limited to bcftools, which is part of the samtools software suite, or varDict can be used to collect SNP information from the final BAM file. Such information concerns the sequence and number of times each variant is present in a sequenced sample was detected and was used to
(a) infer the presence of a genetic mutation, and
(b) to estimate the tumor load using the fetal-fraction estimation/fraction of interest estimation method described in Example 4.

In addition to the detection of the genetic mutation, statistical confidence was ascribed to a detected mutation using the estimated tumor load of the sample and the read-depth of each of the detected variants at a given position using binomial statistics. More than one test may be employed from which one can compute the probability of obtaining the sequenced information, or obtain a 95% confidence interval which describes a range of possible read-depths for the genetic mutation, or whether the obtained proportion of reads which can be ascribed to the genetic mutation is consistent with what would be expected at the given tumor load. A suitable binomial test of proportions is described in Example 4 (in the context of classification of chromosomal abnormalities).

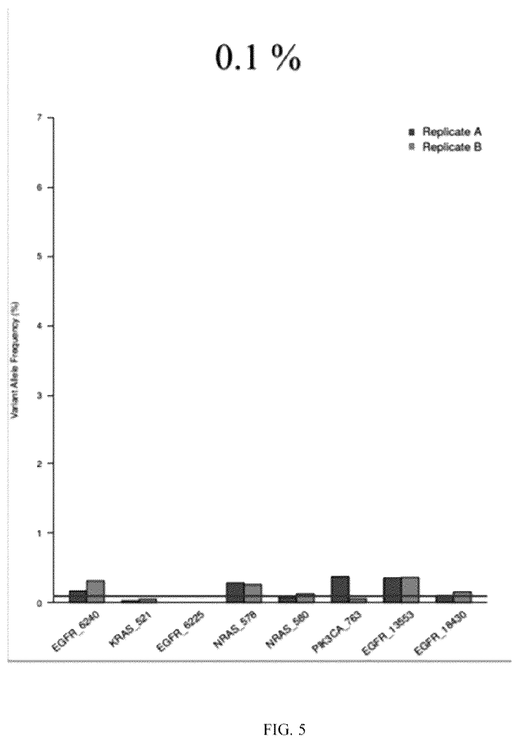

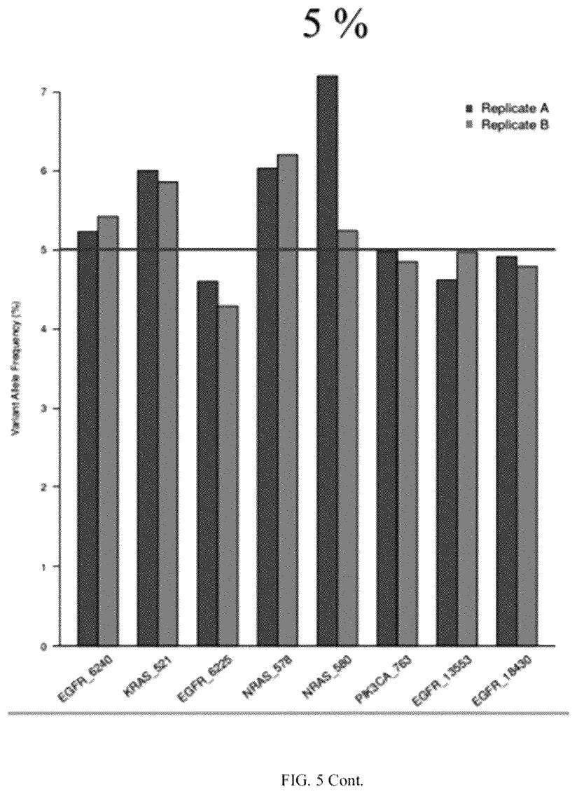

The results are shown in FIG. 5. The line illustrates the expected minor allele frequency (MAF) for each percent (%) tumor load. The bars (x-axis) illustrate the detected MAF (y-axis) for each sample for the indicated genetic mutations. Two technical replicates are shown for the reference material.

The data demonstrates that the TACS methodology successfully detected the tumor-associated genetic mutations EGFR_6240, KRAS_521, EGFR_6225, NRAS_578, NRAS_580, PIK3CA_763, EGFR_13553 and EGFR_18430 at the expected tumor loads of 1.0% and 5.0%. Mutations EGFR_6240, NRAS_578, PIK3CA_763, EGFR_13553 and EFGR_18430 were also successfully detected at 0.1% tumor load.

Accordingly, this example demonstrates the successful detection of a large panel of different tumor biomarkers using the TACS methodology at tumor loads as low as 0.1%.

### Example 7: Tumor Biomarker Detection in Patient Samples

In this example, the TACS methodology, illustrated in FIG. 1, was used for the detection of tumor biomarkers in tumor tissue and blood plasma samples from untreated cancer patients with confirmed diagnosis. For detection of the tumor biomarker sequences of interest, families of TACS, as described in Example 5, were used.

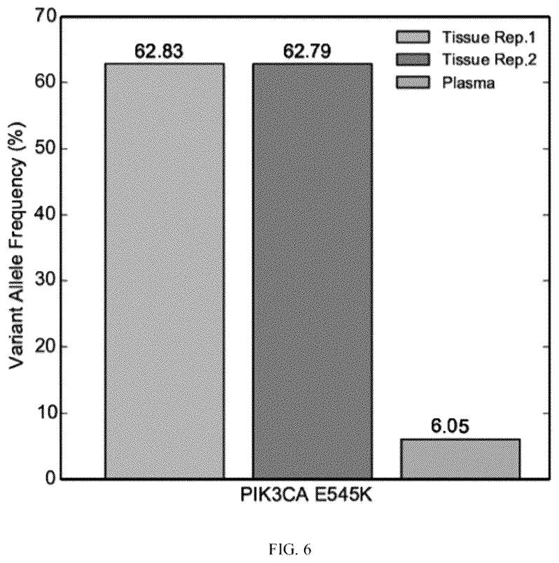

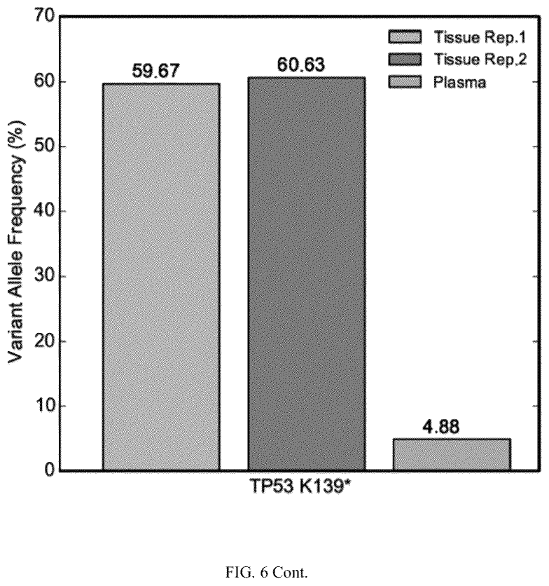

Matched pairs of peripheral blood and tumor tissue samples from untreated cancer patients were used to further validate the performance of the TACS methodology for tumor biomarker detection for a patient harboring mutation PIK3CA E545K (Patient 1) and for a patient harboring mutation TP53 K139 (Patient 2). The results are shown in FIG. 6.

As shown in FIG. 6, application of the TACS methodology to a tissue sample obtained from Patient 1 harboring mutation PIK3CA E545K (top bars) provided a variant allele frequency (VAF) percentage (i.e., the percentage that the genetic mutation is present instead of the normal allele) of ˜62%. Plasma obtained from peripheral blood of Patient 1 was processed according to the method described in Example 1 and provided a 6.05% VAF. Similarly, application of the TACS methodology to samples obtained from Patient 2 harboring mutation TP53 K139 (bottom bars) provided a VAF of ˜60% for tumor tissue and a VAF of 4.88% for plasma obtained from a peripheral blood sample.

Accordingly, this example demonstrates the successful detection of tumor biomarkers in cancer patient samples, in both tumor tissue samples and plasma samples, thereby demonstrating the suitability of the TACS methodology for tissue biopsy and for non-invasive tumor biomarker detection using liquid biopsy.

### Example 8: Detection of Mutational Profiles

Given the ability of the TACS methodology illustrated in FIG. 1 to detect a number of somatic single nucleotide variations (SNVs), these can be examined in the context of motifs, also referred to as mutational profiles. Most somatic mutations in tumors can be considered as passengers and may not be associated with pathogenesis if examined individually. Nonetheless, examining the profile of detected mutations as a whole can be useful in determining and/or detecting a pathogenesis-associated mutational profile. Various algorithms have been developed to decompose known mutational motifs operative in many cancer types. Alternatively, other metrics utilizing specific characteristics such as the type of mutations detected in the context of their neighboring bases can be utilized to this effect. The developed algorithms can infer the most likely scenario(s) that explain the observed data. Decomposition of the number and types of known mutational patterns/signatures that have, most likely, generated the observed mutational profile has been achieved using, but not limited to, the Lawson-Hanson non-negative least squares algorithm.

FIG. 7 shows the observed pattern of somatic SNVs for breast cancer using data downloaded from the COSMIC database. The x-axis shows a single base mutation observed in cancer in the context of its neighboring sequences. For example A[C>A]T describes the mutation of Cytosine (C) to Adenine (A) where the upstream sequence is Adenine and the downstream sequence is Thymine. The y-axis shows the frequency of occurrence of this mutation in breast cancer.

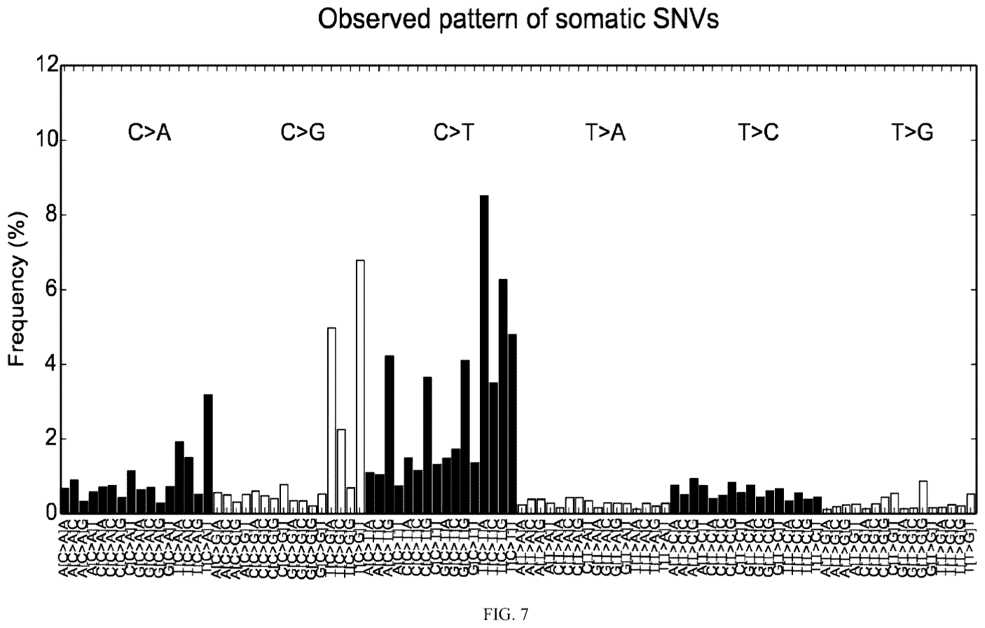

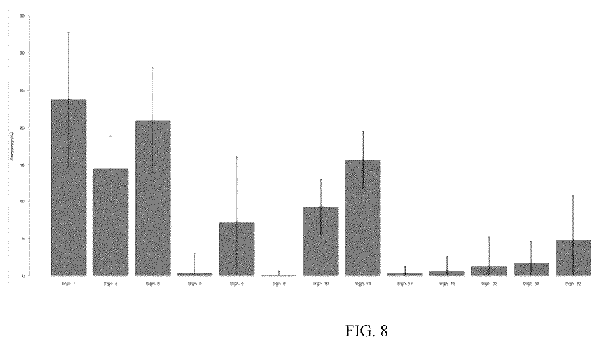

FIG. 8 illustrates the results of a simulations study where mutational profiles were randomly generated by sampling a subset of SNVs each time, from data available in the COSMIC database, thereby simulating individuals. The simulated data were then subjected to the decomposition algorithms described above in order to detect the likely underlying mutational motifs. The bars indicate the average estimated frequency of the known mutational breast signatures computed from a data set of 10000 simulations. The developed algorithm shows evidence of detection of the mutational profiles, thereby demonstrating that detection of mutational profiles, or motifs, is possible using the developed algorithms.

### Example 9: Fragment Size Based Tests

There is evidence from the literature that specific types of cancer can be characterized by and/or associated with fragments in the plasma having a smaller size than the expected size of fragments originating from healthy tissues (Jiang et al, (2015), Proceedings of the National Academy of Sciences, 112(11), ppE1317-E1325). Thus, a fragments-size based test can be utilized to detect the presence of somatic copy number variations in individuals suspected of having cancer. To this effect, a binomial test of proportions, as described Example 4, can be used for the detection of increased presence of nucleic acid material originating from non-healthy tissue (e.g., tumor tissue) based on fragment size. In particular, under the null hypothesis that the distribution of fragment sizes originating from both healthy and non-healthy cells (for example, but not limited to cancerous cells) is the same, a binomial test for proportions (as described in Example 4) using continuity correction can be utilized to quantify any evidence against it.

The same hypothesis holds true for fragments originating from the placenta/fetus. Specifically, placenta derived fragments are generally of smaller size when compared to fragments originating from maternal tissues/cells. Accordingly, assessment of the fragment size-based test was performed using maternal plasma samples (i.e., mixed samples where cell free DNA is of maternal and fetal origin). The size of fragments that have aligned to TACS-enriched regions can be obtained from the aligned data. Subsequently, the proportion of fragments under a specific threshold from a test region is compared respective proportion of fragments from a reference region for evidence against the null hypothesis H0,

H0: The proportion of small fragments of the test-region is not different from the proportion of small-fragments of the reference region.

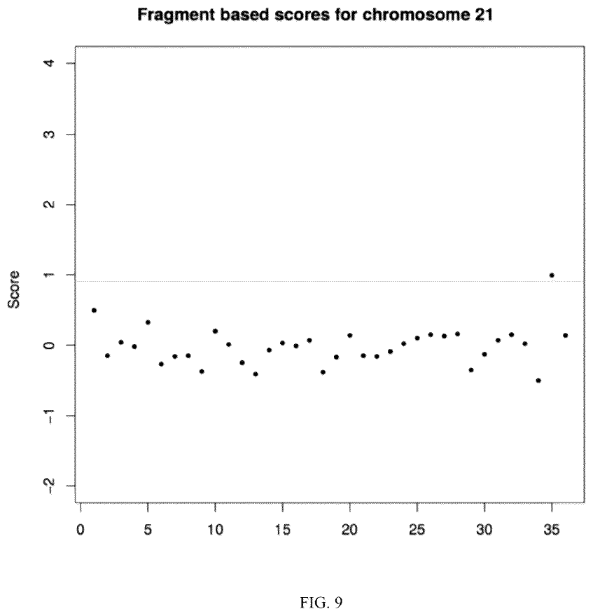

FIG. 9 shows results when applying the fragment sizes method to the mixed sample containing maternal and fetal DNA. The black dots are individual samples. The x-axis shows the sample index. The y-axis shows the score result of the fragments-based method. A score result greater than the one indicated by the threshold, illustrated as a grey line, indicates a deviation from the expected size of fragments illustrating the presence of aneuploidy. The results demonstrate that an aneuploid sample, having an estimated fetal fraction equal to 2.8%, was correctly identified, illustrating that fragments-based detection may be used to detect abnormalities in mixed samples with low signal-to-noise ratio (e.g., as is the case in detection of cancer).

Accordingly, this example demonstrates the successful ability of the fragments-based detection method in detecting genetic abnormalities in mixed samples with low signal-to-noise ratios, thereby demonstrating the suitability of the fragments-based test for analysis of either cancer samples for oncology purposes or maternal samples for NIPT.

Since small-sized fragments are associated with fragments from non-healthy tissues (Jiang et al, (2015), Proceedings of the National Academy of Sciences, 112(11), ppE1317-E1325) they can also be leveraged for the detection of small-sized mutations, such as point mutations and mutational signatures. For example, one may only use small-sized fragments in Variant Allele Frequency estimation as described in examples 6-9, thereby increasing the signal-to-noise ratio.

### Example 10: Use of the Method for Tissue Biopsies

Five FFPE samples from Breast carcinoma and 13 tissue samples (fresh/frozen and FFPE) from lung adenocarcinoma were subjected to the method and the mutational status was successfully detected. The data are presented below.

### Breast Carcinoma:

| Type of | | | | | | |
|---|---|---|---|---|---|---|
| specimen | Patient ID | Gene | CDS mutation | AA change | COMSIC ID | MAF % |
| FFPE | BCa1 | TP53 | c.569delC | p.190fs*57 | COSM100030 | 17.39 |
| | | TP53 | c.559 + 25G > A | intronic | COSM45841 | 22.22 |
| FFPE | BCa2 | PIK3CA | c.1624G > A | p.E542K | COSM760 | 32.15 |
| | | FLT3 | c.2501G > A | p.R834Q | COSM28047 | 2.01 |
| FFPE | BCa3 | ALK | c.3515 + 18C > T | intronic | COSM28496 | 49.43 |
| FFPE | BCa4 | AKT1 | c.49G > A | p.E17K | COSM33765 | 42.86 |
| FFPE | BCa5 | PIK3CA | c.1633G > A | E545K | COSM763 | 62.79 |
| | | PTEN | c.1-9C > G | intronic | COSM5915 | 26.19 |
| | | TP53 | c.415A > T | p.K139* | COSM44678 | 60.63 |

### Lung Adenocarcinoma:

| | | | NIPD | Independent |
|---|---|---|---|---|
| Type of specimen | Sample ID | COSMIC ID | method | method |
| Fresh/frozen | LCA1 | COSM459 | 0.0245 | 0.0261 |
| | | COSM527 | 0.036 | 0.0378 |
| Fresh/frozen | LCA2 | COSM763 | 0.1427 | 0.1558 |
| Fresh/frozen | LCA3 | COSM522 | 0.3815 | 0.3492 |
| Fresh/frozen | LCA4 | COSM521 | 0.106 | 0.0923 |
| Fresh/frozen | LCA6 | COSM3675521 | 0.1387 | 0.1026 |
| Fresh/frozen | LCA13 | MET | 0.0126 | Not covered |
| | | c.3028 + 1G > T | | |
| Fresh/frozen | LCA15 | COSM27887 | 0.1352 | Not covered |
| | | COSM521 | 0.2871 | 0.3185 |
| Fresh/frozen | LCA21 | COSM763 | 0.2798 | 0.3128 |
| FFPE | LCA36 | COSM6224 | 0.2431 | 0.1847 |
| FFPE | LCA40 | COSM6225 | 0.08 | 0.1148 |
| FFPE | LCA45 | COSM6223 | 0.2098 | 0.2456 |
| FFPE | LCA47 | COSM12370 | 0.6295 | 0.5719 |
| FFPE | LCA48 | COSM522 | 0.2649 | 0.4032 |

For the lung cancer data, results were compared with data obtained for the same tissue samples with an independent method. For the genomic regions covered by both methods we observed 100% concordance.

1. A method of diagnosing cancer in a subject suspected of having cancer based on the detection of at least one tumor biomarker sequence in a DNA sample from said subject, wherein the DNA sample is a blood sample or urine sample, and comprises cell free tumor DNA (cftDNA), wherein the method comprises:
(a) preparing a sequencing library from the DNA sample;
(b) hybridizing the sequencing library to a pool of double-stranded TArget Capture Sequences (TACS) that bind to one or more tumor biomarker sequences of interest,
wherein:
(i) each member sequence within the pool of TACS is between 100-500 base pairs in length, each member sequence having a 5′ end and a 3′ end;
(ii) each member sequence binds to the tumor biomarker sequence of interest at least 50 base pairs away, on both the 5′ end and the 3′ end, from regions harboring Copy Number Variations (CNVs), Segmental duplications or repetitive DNA elements; and
(iii) the GC content of the pool of TACS is between 19% and 80%, as determined by calculating the GC content of each member within the pool of TACS;
(c) isolating members of the sequencing library that bind to the pool of TACS to obtain an enriched library;
(d) amplifying and sequencing the enriched library; and
(e) analyzing the enriched library sequences for the presence or absence of the at least one tumor biomarker sequence, thereby diagnosing the patient.

2. The method according to claim 1, wherein the pool of TACS comprises a plurality of TACS families directed to different tumor biomarker sequences of interest, wherein each TACS family comprises a plurality of member sequences, wherein each member sequence binds to the same tumor biomarker sequence of interest but has different start and/or stop positions with respect to a reference coordinate system for the genomic sequence of interest.

3. The method according to claim 2, wherein the pool of TACS comprises at least 50 different TACS families.

4. The method according to claim 2, wherein each TACS family comprises at least 3 member sequences.

5. The method according to claim 2, wherein the start and/or stop positions for the member sequences within a TACS family, with respect to a reference coordinate system for the genomic sequence of interest, are staggered by at least 3 base pairs.

6. The method according to claim 1, wherein the TACS are biotinylated and are bound to streptavidin-coated magnetic beads.

7. The method according to claim 1, wherein the pool of TACS binds to at least one tumor biomarker sequence selected from the genes listed in table 1.

8. The method according to claim 1, wherein amplification of the enriched library is performed in the presence of blocking sequences that inhibit amplification of wild-type sequences.

9. The method according to claim 1, further comprising determining and/or detecting mutational profiles.

10. A kit comprising a container comprising a pool of TArget Capture Sequences (TACS),
wherein the pool of TACS comprises a plurality of TACS families,
wherein each TACS family comprises a plurality of member sequences,
wherein each member sequence binds to the same genomic sequence of interest but has different start and/or stop positions with respect to a reference coordinate system for the genomic sequence of interest, and further wherein:
(i) each member sequence within each TACS family is between 100-500 base pairs in length, each member sequence having a 5′ end and a 3′ end;
(ii) each member sequence binds to the same genomic sequence of interest at least 50 base pairs away, on both the 5′ end and the 3′ end, from regions harboring Copy Number Variations (CNVs), Segmental duplications or repetitive DNA elements; and
the GC content of the pool of TACS is between 19% and 80%, as determined by calculating the GC content of each member within each family of TACS.
